(19) United States
(12) Patent Application Publication
(10) Pub. No.: US 2026/0308235 A1
(43) Pub. Date: Oct. 8, 2026

(54) Chimeric Cytokine Receptors and Methods of Use

(71) Applicant: The Board of Trustees of the Leland Stanford Junior University, Stanford, CA (US)

(72) Inventors: Louai Labanieh, Redwood City, CA (US); Crystal L. Mackall, Redwood City, CA (US); Robbie G. Majzner, Redwood City, CA (US)

(21) Appl. No.: 19/104,149

(22) PCT Filed: Aug. 25, 2023
(86) PCT No.: PCT/US2023/072943

§ 371 Date: Feb. 14, 2025

Related U.S. Application Data
(60) Provisional application No. 63/401,437, filed on Aug. 26, 2022.

(51) Int. Cl. C07K 14/715; A61K 40/11; A61K 40/31; A61K 40/42; A61P 35/02; C07K 16/32; C12N 5/0783; C12N 9/50

(52) U.S. Cl. CPC C07K 14/7155; A61K 40/11; A61K 40/31; A61K 40/4205; A61K 40/4217; A61P 35/02; C07K 16/32; C12N 5/0636; C12N 9/506; C12Y 304/21098; C07K 2319/02; C07K 2319/03; C07K 2319/42; C07K 2319/43; C07K 2319/50; C07K 2319/73; C12N 2510/00

(57) ABSTRACT

Provided are nucleic acids encoding chimeric cytokine receptors (CCRs) capable of signaling in the absence of their cognate cytokines. In some embodiments, provided are one or more nucleic acids encoding a first subunit of a chimeric cytokine receptor and a second subunit of the chimeric cytokine receptor. The first subunit comprises a first heterologous dimerization domain and a first cytokine receptor intracellular signaling domain (ICD). The second subunit comprises a second heterologous dimerization domain cognate for the first heterologous dimerization domain, and a second cytokine receptor ICD. The CCRs find use in a variety of contexts, including but not limited to, increasing the persistence of therapeutic cells. Accordingly, also provided are methods of administering a cell-based therapy, the methods comprising administering therapeutic cells (e.g., CAR-T cells, etc.) expressing the CCRs to a subject in need thereof.

### CROSS-REFERENCE TO RELATED APPLICATIONS

This application claims the benefit of U.S. Provisional Patent Application No. 63/401,437, filed Aug. 26, 2022, which application is incorporated herein by reference in its entirety.

### INCORPORATION BY REFERENCE OF SEQUENCE LISTING PROVIDED AS A SEQUENCE LISTING XML FILE

A Sequence Listing is provided herewith as a Sequence Listing XML, “STAN-1985WO_SEQ_LIST”, created on Aug. 24, 2023 and having a size of 109,370 bytes. The contents of the Sequence Listing XML are incorporated herein by reference in their entirety.

### INTRODUCTION

Cytokines are intercellular signaling molecules that aid cell to cell communication in immune responses and stimulate the movement of cells towards sites of inflammation, infection and trauma. The downstream effect of a particular cytokine occurs through its high-affinity binding of its receptor expressed on the surface of a target cell. This action may occur in an autocrine (acts on the same cell), paracrine (acts on nearby cells) or endocrine (acts on distant cells) manner. Receptor engagement triggers intracellular signaling cascades leading to altered gene expression in the target cell, leading to a biological effect. Cytokines can be divided into several categories including the interleukins (ILs), transforming growth factors (TGFs), interferons (IFNs), colony-stimulating-factors (CSFs), tumor necrosis factors (TNFs), and chemokines.

Interleukins (ILs) are a group of cytokines that are expressed and secreted by white blood cells (leukocytes) as well as some other body cells. Interleukins and associated cytokines serve as the means of communication for innate and adaptive immune cells as well as non-immune cells and tissues. All IL-1 family members share a conserved beta-trefoil structure and bind to members of the IL-1 receptor (IL-1R) family. Members of the IL-1R family contain extracellular Ig-like domains and mediate signaling through an intracellular Toll/IL-1R (TIR) domain.

The four-helix bundle cytokine superfamily is subdivided into the class I and class II cytokine receptor families. Ligands for the class I cytokine receptor family include short-chain and long-chain helical cytokines. The short-chain helical cytokine family includes members of the common gamma-chain and common beta-chain families of cytokines. The common beta-chain and common gamma-chain cytokine families include cytokines such as IL-2, IL-3, IL-4, IL-5, IL-7, IL-9, IL-15, IL-21, and GM-CSF. Members of the common beta-chain family signal through heterodimeric receptor complexes that contain the common beta-chain subunit, while members of the common gamma-chain family signal through heterodimeric or heterotrimeric receptor complexes that contain the common gamma-chain subunit. The common γ chain (γc) family consists of IL-2, IL-4, IL-7, IL-9, IL-15, and IL-21 and was named for binding of these factors to the γc receptor (CD132). They act mainly as growth and proliferation factors for progenitors and mature cells and also have roles in lineage-specific cell differentiation.

Ligands for the class II cytokine receptor family include the IL-10 family cytokines, along with the type I, type II, and type III interferons. Members of the IL-10 family cytokines have structural similarities and signal through heterodimeric receptor complexes with common subunits. Members of the type I, II, and Ill interferon families include IFN-alpha, IFN-beta, IFN-omega, IFN-epsilon, IFN-kappa, IFN-gamma, IL-28A, IL-28B, IL-29, and IFN-lambda4. These cytokines primarily have anti-viral, anti-proliferative, and immunoregulatory effects.

The IL-17 family cytokines belong to the cysteine-knot superfamily and bind to members of the IL-17 receptor family. They are primarily involved in promoting pro-inflammatory immune responses.

Members of the tumor necrosis factor (TNF) superfamily form homotrimers or in some cases, heterotrimers, and share a common extracellular domain known as the TNF homology domain (THD). Cytokines in the TNF superfamily bind to oligomeric, type I or type II transmembrane proteins that have multiple extracellular cysteine-rich domains. Many members of the TNF superfamily regulate apoptosis and/or immune cell functions, such as T cell co-stimulation, natural killer cell activation, and B cell homeostasis. Additionally, they can regulate cell type-specific responses and can play a significant role in regulating the pathogenesis of certain diseases, including chronic inflammation, cancer, and autoimmune diseases.

The four short-chain helix bundle cytokines that signal through class III receptor tyrosine kinases include M-CSF, SCF, Flt-3 Ligand, and IL-34. The receptors for these cytokines contain extracellular Ig-like domains, similar to the IL-1R family, but they have cytoplasmic domains with tyrosine kinase activity. The long-chain helical cytokine family includes the IL-6 family cytokines, G-CSF, erythropoietin, thrombopoietin, growth hormone, prolactin, and leptin.

The four subfamilies of chemokines are the C, CX3C, CC, and CXC subfamilies, and are based on the number and spacing of conserved cysteine residues located in the amino terminus. Chemokines bind to conventional G protein-coupled seven transmembrane receptors, with some promiscuity, and have a key role in regulating cell migration during development, and under homeostatic and inflammatory conditions.

### SUMMARY

Provided are nucleic acids encoding chimeric cytokine receptors (CCRs) capable of signaling in the absence of their cognate cytokines. In some embodiments, provided are one or more nucleic acids encoding a first subunit of a chimeric cytokine receptor and a second subunit of the chimeric cytokine receptor. The first subunit comprises a first heterologous dimerization domain and a first cytokine receptor intracellular signaling domain (ICD). The second subunit comprises a second heterologous dimerization domain cognate for the first heterologous dimerization domain, and a second cytokine receptor ICD. According to some embodiments, signaling by the CCRs is regulatable, e.g., using a regulatable protease wherein, in the absence of an inhibitor of the protease, an ICD is cleaved from one or both subunits of a CCR thereby preventing signaling by the CCR. The CCRs find use in a variety of contexts, including but not limited to, increasing the persistence of therapeutic cells. Accordingly, also provided are methods of administering a cell-based therapy, the methods comprising administering therapeutic cells (e.g., CAR-T cells, etc.) expressing the CCRs to a subject in need thereof.

### BRIEF DESCRIPTION OF THE FIGURES

FIG. 1: A: Receptors of the IL-2 family, which is composed of IL-2, IL-4, IL-7, IL-9, IL-15, and IL-21. Receptors contain the common cytokine receptor γ chain (CD132, γc). IL-13R shares IL-4Rα with IL-4, and TSLPR shares IL-7R with IL-7. B: Receptors for IL-3, IL-5, and GM-CSF are heterodimers of a unique a chain and the common β chain (βc, CD131) subunit. C: Receptors for IL-4 and IL-13 consist of 2 receptor chains: IL-4Rα (CD124) and γc. IL-4 and IL-13 bind to IL-4R, which consists of IL-4Rα and the IL-13Rα1 chain. IL-13R consists of 2 subunits, IL-13Rα1 and IL-13Rα2, and signaling occurs through the IL-4R complex type II, which consists of IL-4Rα and IL-13Rα. D: Based on similarities in their intron-exon structure, conserved secondary protein structures, and similar types of receptors, the following cytokines have been classified as IL-10 family members: IL-10, IL-19, IL-20, IL-22, IL-24, IL-26, IL-28, and IL-29. They share common receptor subunits, as shown. E: TNF-α binds to TNFRI and TNFR2, and TGF-β binds to heterodimer receptor consisting of TGF-βR1 and TGF-βR2. F: IL-12R consists of 2 subunits: IL-12Rβ1 and IL-12Rβ3. A heterodimer of IL-12Rβ1 and IL-23R binds IL-23. IL-12Rβ2 shows homology to the gp130 subunit of IL-27R. G: IFN-α and IFN-β bind to the heterodimer receptor consisting of IFNAR1 and IFNAR2; in addition, IFN-β binds to IFNAR1, and IFN-γ binds to the IFN-γR1 and IFN-γR2 heterodimer. Adapted from Akdis et al. (2016) J Allergy Clin Immunol. 138(4): 984-1010.

FIG. 2: Schematic illustration of leucine zipper based chimeric cytokine receptors (CCRs).

FIG. 3: Flow plots showing cell surface expression of the CCR components for various common gamma chain receptors.

FIG. 4: Schematic illustration of CCR engineering (depicted transition domains in γC1/γC3 and γC2/γC4 are SEQ ID NOs: 54 and 55, respectively) and flow plots demonstrating improved cell surface expression of CCRs.

FIG. 5: A plot showing proliferation of primary human T cells transduced with the various CCRs shown in FIG. 4.

FIG. 6: Data demonstrating that a constitutive IL-2 CCR is a strong driver of proliferation in vivo and results in lethal toxicity in NSG mice.

FIG. 7: A schematic illustration of a drug-regulated chimeric cytokine receptor (CCR) system. A protease cleavage site is integrated between the transmembrane domain and intracellular signaling domain of the CCR. Co-expression of a protease that is specific for the cleavage site results in cleavage of the CCR due to proteolytic activity of the protease at the cleavage site, resulting in inactivation of the CCR (receptor OFF). Addition of a protease inhibitor (drug) inhibits this cleavage event and renders the CCR in the on state (Receptor ON). Thus, signaling of the CCR becomes dependent upon the presence of the drug. The regulated CCR could circumvent toxicities associated with constitutive CCRs by regulating proliferation of cells to fall within a therapeutic window.

FIG. 8: A series of flow plots of primary human T cells transduced with regulated versions of the IL-2R CCR, as described in FIG. 7, demonstrating cell surface expression of HA/FLAG tag and phosphorylated STAT5 (pSTAT5), which is a cytokine signaling molecule downstream of IL-2R signaling (depicted cleavage site is SEQ ID NO:56).

FIG. 9: Amino acid sequences and data demonstrating that the insertion of a linker containing a cleavage site (SEQ ID NO:57) upstream of gamma chain ICD allows for drug regulated CCR signaling.

FIG. 10: Flow plots of primary human T cells transduced with regulated versions of the IL-2R CCR, demonstrating phosphorylated STAT5 (pSTAT5).

FIG. 11: Data demonstrating drug control of cell expansion in vitro.

FIG. 12: Schematic illustration of CAR and CCR constructs and data demonstrating that drug regulated IL-2 CCR enhances in vivo antitumor activity following rechallenge.

FIG. 13: Schematic illustration of CAR and CCR constructs and data demonstrating that drug regulated IL-2 CCR expands CAR-T cells in vivo without toxicity.

FIG. 14: Schematic illustration of CAR and CCR constructs and data demonstrating that CAR-T cells expressing the drug regulated IL-2 CCR from two separate vectors have enhanced expansion in vivo in the presence of grazoprevir.

FIG. 15: A flow plot showing phosphorylation levels of STAT5 from primary human T cells transduced with an IL-2 CCR with (leucine zipper IL-2 CCR) or without (CD8a H/Tm IL-2 CCR) the leucine zipper components.

FIG. 16: A flow plot for an IL-21R CCR demonstrating phosphorylation of STAT3 (pSTAT3), which is a cytokine signaling molecule downstream of IL-21R signaling.

FIG. 17: A flow plot for constitutive or protease regulated IL-2R, IL-7R and IL-9R CCRs expressed in primary human T cells.

FIG. 18: Data demonstrating that constitutive IL-2 CCR, IL-9 CCR, and IL-7 CCR induce lethal toxicity in mice due to the unregulated outgrowth of CAR-T cells.

FIG. 19: Data demonstrating controlled growth of CAR-T cells expressing protease regulated CCRs.

FIG. 20: Schematic illustration of an approach to reduce “leaky” activity of protease regulated CCRs by dual chain cleavage. A: a schematic of the original drug-regulated chimeric cytokine receptor (CCR) system. A protease cleavage site is integrated between the transmembrane domain and intracellular signaling domain of one of the chains of the CCR. Co-expression of a protease that is specific for the cleavage site results in cleavage of the CCR due to proteolytic activity of the protease at the cleavage site, resulting in inactivation of the CCR (receptor OFF). Addition of a protease inhibitor (drug) inhibits this cleavage event and renders the CCR in the on state (Receptor ON). Thus, signaling of the CCR becomes dependent upon the presence of the drug. The regulated CCR could circumvent toxicities associated with constitutive CCRs by regulating proliferation of cells to fall within a therapeutic window. B: a schematic of the dual cleavage drug-regulated chimeric cytokine receptor (CCR) system, whereby both chains of the CCR are cleaved. A protease cleavage site is integrated between the transmembrane domain and intracellular signaling domain of both chains/subunits of the CCR. Co-expression of a protease that is specific for the cleavage site results in cleavage of the CCR due to proteolytic activity of the protease at the cleavage site, resulting in inactivation of the CCR (receptor OFF). Addition of a protease inhibitor (drug) inhibits this cleavage event and renders the CCR in the on state (receptor ON). Thus, signaling of the CCR becomes dependent upon the presence of the drug. The regulated CCR is expected to circumvent toxicities associated with constitutive CCRs by regulating proliferation of cells to fall within a therapeutic window.

FIG. 21: A: Schematic illustration for a regulatable CCR with a protease fused to one of the CCR subunits. B: A flow plot showing phosphorylation levels of STAT5 from primary human T cells transduced with an IL-2 CCR with a HCV NS3 protease directly fused to the gamma chain ICD (“cis”). A short or long peptide linker was placed between the gamma chain ICD and the protease. “Trans” protease refers to the original protease configuration as shown in FIG. 7.T cells were cultured in media lacking IL2 for 24 hours prior to analysis of STAT5 phosphorylation levels.

FIG. 22: A flow plot showing CD22.BBz CAR surface expression levels from primary human T cells transduced with various CCRs. The data demonstrate that all configurations result in similar levels of surface CAR expression. “Constit.” and “Reg.” refer to constitutive and regulatable CCRs, respectively.

FIG. 23: NSG mice were inoculated with Nalm6 leukemia then treated with CD22.BBz CAR-T cells engineered with various IL2CCR constructs. GPV was administered to mice daily (++), every 48 hours (+), or not at all (−). Mock untransduced T cells, conventional CD22.BBz CAR-T cells, and CD22.BBz CAR-T cells engineered with a CCR that lacks ICDs (ΔICD) serve as controls. A: Quantification of tumor progression B: Quantification of T cell expansion.

FIG. 24: NSG mice were inoculated with Nalm6 leukemia then treated with CD22.BBz CAR-T cells engineered with various IL-9 CCR constructs. Mock untransduced T cells and conventional CD22.BBz CAR-T cells serve as controls. A: Quantification of tumor progression B: Quantification of T cell expansion.

FIG. 25: Data demonstrating drug control of cell expansion of CD22.BBz CAR-T cells in vitro. T cells were cultured in media lacking IL-2.

FIG. 26: Data demonstrating drug control of pSTAT5 levels of HER2.BBz CAR-T cells in vitro. T cells were cultured in media lacking IL-2.

FIG. 27: NSG mice were inoculated with Nalm6 leukemia then treated with CD22.BBz CAR-T cells engineered with only the IL-2RB component of the IL2 CCR. Mock untransduced T cells and conventional CD22.BBz CAR-T cells serve as controls. The data show quantification of tumor progression.

FIG. 28: A: Flow plots demonstrating cell surface expression of CCRs with IL-18RA and IL-18RB ICDs B: NSG mice were inoculated with Nalm6 leukemia then treated with CD22.BBz CAR-T cells engineered with Regulated or constitutive IL-18CCR constructs. For regulated IL-18CCR groups, GPV was administered to mice daily (+GPV), or not at all (−GPV). Mock untransduced T cells and CD22.BBz CAR-T cells engineered with a CCR that lacks ICDs (ΔICD) serve as controls. The data show quantification of tumor progression.

### DETAILED DESCRIPTION

Before the nucleic acids, chimeric cytokine receptors and methods of the present disclosure are described in greater detail, it is to be understood that the nucleic acids, chimeric cytokine receptors and methods are not limited to particular embodiments described, as such may, of course, vary. It is also to be understood that the terminology used herein is for the purpose of describing particular embodiments only, and is not intended to be limiting, since the scope of the nucleic acids, chimeric cytokine receptors will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the nucleic acids, chimeric cytokine receptors and methods. The upper and lower limits of these smaller ranges may independently be included in the smaller ranges and are also encompassed within the nucleic acids, chimeric cytokine receptors and methods, subject to any specifically excluded limit in the stated range. Where the stated range includes one or both of the limits, ranges excluding either or both of those included limits are also included in the nucleic acids, chimeric cytokine receptors and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the nucleic acids, chimeric cytokine receptors and methods belong. Although any nucleic acids, chimeric cytokine receptors and methods similar or equivalent to those described herein can also be used in the practice or testing of the nucleic acids, chimeric cytokine receptors and methods, representative illustrative nucleic acids, chimeric cytokine receptors and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present nucleic acids, chimeric cytokine receptors and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the nucleic acids, chimeric cytokine receptors and methods, which are, for clarity, described in the context of separate embodiments, may also be provided in combination in a single embodiment. Conversely, various features of the nucleic acids, chimeric cytokine receptors and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present nucleic acids, chimeric cytokine receptors and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Chimeric Cytokine Receptors and Nucleic Acids

Aspects of the present disclosure include chimeric cytokine receptors (CCRs) and nucleic acids encoding same. The CCR platform of the present disclosure allows for constitutive or regulatable signaling of cytokine receptors in the absence of their cognate cytokines. The CCRs find use in a variety of contexts. By way of example, cells that express a CCR of the present disclosure find use in cell therapy. Cell therapies such as CAR-T cell therapy have demonstrated remarkable clinical benefit in hematologic malignancies. However, T cell dysfunction and poor persistence limit clinical responses, particularly in solid tumors. As demonstrated herein, when expressed on cells, the CCRs of the present disclosure are strong drivers of cell proliferation in vitro and in vivo, and provide for enhanced in vivo anti-tumor activity as compared to control cells lacking the CCR. Accordingly, the CCRs and cells expressing the same find use in cell therapy, non-limiting examples of which include immune cell therapy (e.g., immune cells (e.g., tumor-infiltrating lymphocytes) lacking an antigen-binding receptor), CAR-T cell therapy, TCR therapy, CAR-NK cell therapy, and the like. Details regarding the CCRs and nucleic acids of the present disclosure will now be described.

In certain embodiments, provided are one or more nucleic acids encoding a first subunit of a chimeric cytokine receptor and a second subunit of the chimeric cytokine receptor. As used herein, the term “chimeric” refers to a molecule, e.g., a cytokine receptor, composed of parts of different origins. A chimeric molecule, as a whole, is non-naturally occurring, e.g., synthetic or recombinant, although the parts which comprise the chimeric molecule can be naturally occurring. By “chimeric cytokine receptor” or “CCR” is meant a cytokine receptor comprising one or more subunits comprising one or more heterologous domains, where the receptor is capable of constitutive or regulatable signaling in the absence of its cognate cytokine(s). “Heterologous” as used in the context of a nucleic acid or polypeptide domain generally means that the domain is from a different origin (e.g., molecule of different sequence, different species origin, and/or the like) than that with which the nucleic acid or polypeptide is associated or joined, such that the nucleic acid or polypeptide is one that is not found in nature.

The CCR may be a chimeric version of any cytokine receptor of interest (e.g., any human cytokine receptor of interest), where the CCR is capable of constitutive or regulatable signaling in the absence of its cognate cytokine(s). In certain embodiments, the CCR is a chimeric version of a member of the class I cytokine receptor family, a chimeric version of a member of the class II cytokine receptor family, a chimeric version of a member of the TNF receptor family, a chimeric version of a member of the IL-1 receptor family, a chimeric version of a member of the tyrosine kinase receptor family, or a chimeric version of a member of the chemokine receptor family.

According to some embodiments, the first and second subunits of the CCR are chimeric versions of first and second subunits (e.g., human subunits) of an interleukin (IL) receptor, a transforming growth factor (TGF) receptor, an interferon (IFN) receptor, a colony-stimulating-factor (CSF) receptor, a tumor necrosis factor (TNF) receptor, or a chemokine receptor.

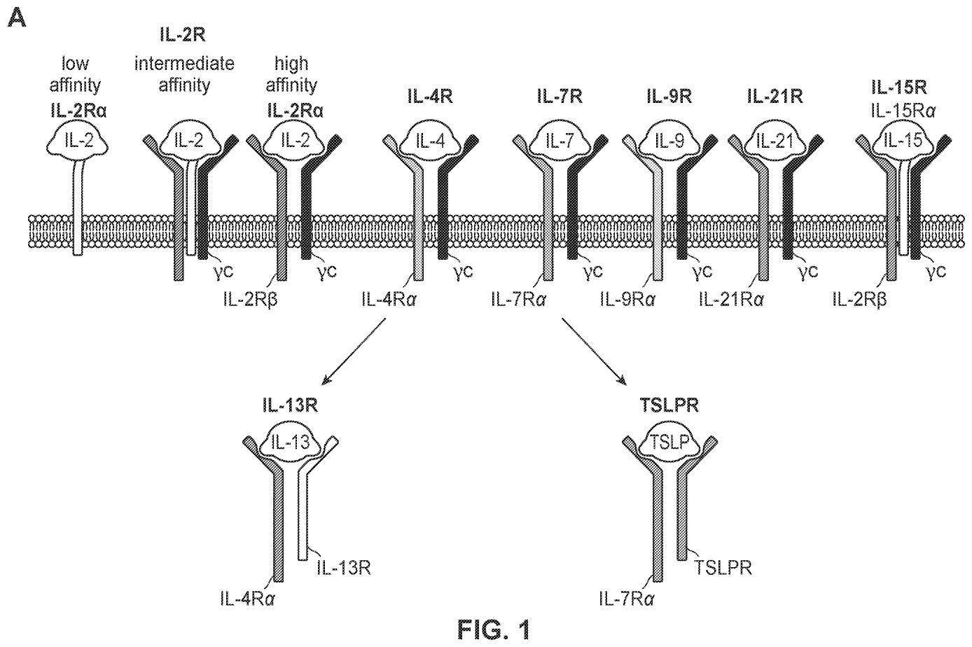

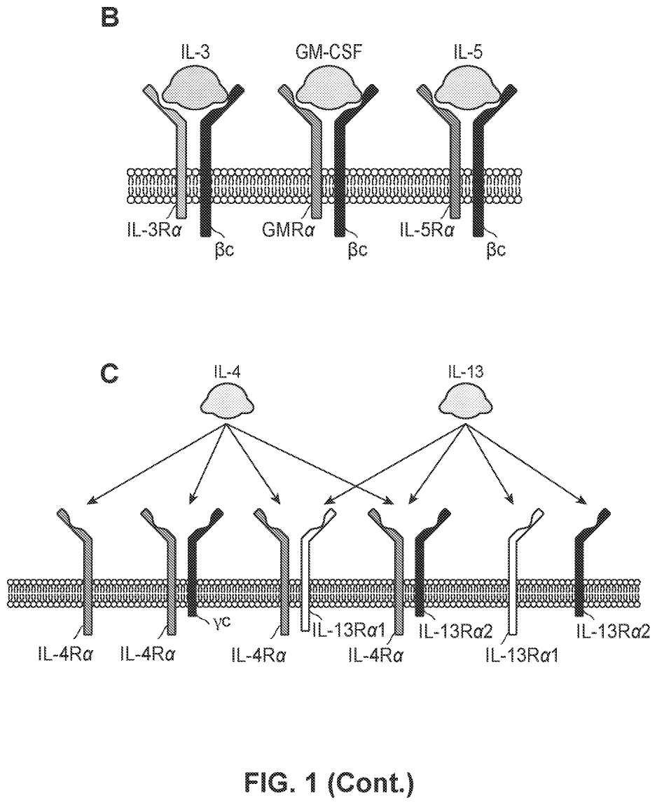

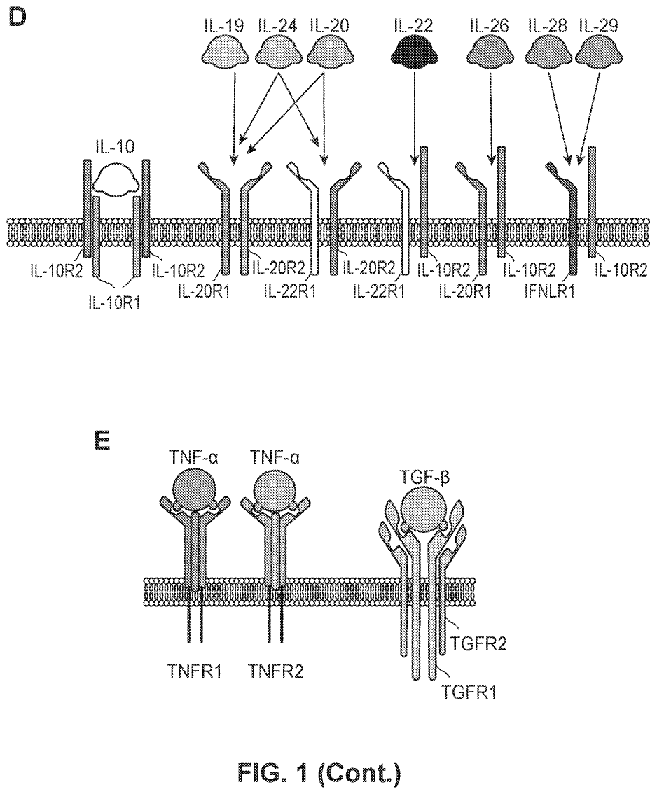

In certain embodiments, the first and second subunits of the CCR are chimeric versions of first and second subunits independently selected from any of the cytokine receptor subunits shown in FIG. 1.

According to some embodiments, the first and second subunits of the CCR are chimeric versions of first and second subunits of receptor (e.g., a human receptor) for IL-2, IL-3, IL-4, IL-5, IL-7, IL-9, IL-10, IL-12, IL-13, IL-15, IL-19, IL-20, IL-21, IL-22, IL-23, IL-24, IL-26, IL-27, IL-28, IL-29, IL-35, TSLP, GM-CSF, TNFα, TGFβ, IFNα, IFNβ. Subunits of receptors for those cytokines are shown in FIG. 1.

The amino acid sequences of cytokine receptor subunits (e.g., human cytokine receptor subunits) and ICDs thereof, as well as suitable nucleotide sequences for encoding and expressing such subunits and ICDs thereof, are known and readily available from databases such as UniProt, NCBI Protein, GenBank and others. Non-limiting examples include human IL-2RB (UniProt KB—P14784), human IL-4Rα (UniProt KB—P24394), human IL-7Rα (UniProt KB—P16871), human IL-9R (UniProt KB—Q01113), human IL-10Rβ (UniProt KB—Q08334), human IL-12Rβ1 (UniProt KB—P42701), human IL-21R (UniProt KB—Q9HBE5), human IL-22Rα1 (UniProt KB—Q8N6P7), human IL-23R (UniProt KB—Q5VWK5) and human common gamma chain (γc) (UniProt KB P31785).

In certain embodiments, a CCR of the present disclosure retains binding activity for its cognate cytokine(s). For example, the CCR may comprise the extracellular cytokine-binding portion of the cytokine receptor found in nature, or a variant thereof that retains binding activity for the cognate cytokine(s). According to some embodiments, a CCR of the present disclosure is engineered so that the extracellular cytokine-binding portion of the cytokine receptor found in nature is absent or modified in such a way that the CCR no longer retains binding activity for its cognate cytokine(s).

According to some embodiments, the CCRs of the present disclosure comprise a first subunit comprising a first heterologous dimerization domain and a first cytokine receptor intracellular signaling domain (ICD), and a second subunit comprising a second heterologous dimerization domain cognate for the first heterologous dimerization domain, and a second cytokine receptor ICD. By “cognate” in this context is meant that the second dimerization domain, when the first and second subunits are co-expressed on the surface of cell, is capable of dimerizing with the first dimerization domain, in turn resulting in the formation of a dimer comprising the first and second subunits. Upon co-expression of the first and second subunits on the surface of a cell, the first and second heterologous dimerization domains may be extracellular dimerization domains, transmembrane dimerization domains, or intracellular dimerization domains.

Any suitable heterologous dimerization domains may be employed in the first and second subunits of a CCR. In certain embodiments, the first and second heterologous dimerization domains each comprise a leucine zipper domain, a BTB (BR-C, ttk and bab) domain, a POZ (Pox virus and Zinc finger) domain, a coiled coil domain, or a PDZ domain. According to some embodiments, the first and second heterologous dimerization domains comprise, respectively, a constant heavy chain and constant light chain of an IgG, IgE, or IgD antibody, or dimerizing fragments thereof.

According to some embodiments, upon co-expression of the first and second subunits on the surface of a cell, the first and second heterologous dimerization domains are transmembrane dimerization domains. For example, the first subunit of the CCR may comprise a heterologous transmembrane domain which is the first heterologous dimerization domain, and the second subunit of the CCR may comprise a heterologous transmembrane domain which is the second heterologous dimerization domain. Examples of such heterologous transmembrane domains include, but are not limited to, CD8α (UniProt—P01732) transmembrane domains, CD28 (UniProt—P10747) transmembrane domains, HER2 (UniProt—Q9UK79), EGFR (UniProt—P00533), or the like.

In certain embodiments, the first and second subunits comprise a transmembrane domain independently selected from a HER2, EGFR, IL-2Rβ, IL-7R, IL-21R, IL-4R, IL-9R, IL15Rα, common gamma chain (γc), Eph receptor, VEGF receptor, ErbB receptor, FGF receptor, ROR1, ROR2 PDGF receptor, MET receptor, CD35, CD3ζ, CD3γ, CD3δ, CD4, CD5, CD9, CD16, CD22, CD27, CD28, CD33, CD37, CD45, CD64, CD80, CD86, CD134, CD137, CD152, CD154, and PD-1 transmembrane domain. The amino acid sequences of such receptors (e.g., human receptors) and transmembrane domains thereof, as well as suitable nucleotide sequences for encoding and expressing such transmembrane domains, are known and readily available from databases such as UniProt, NCBI Protein, GenBank and others.

As summarized above, the CCRs of the present disclosure comprise first and second subunits comprising first and second cytokine receptor intracellular signaling domains (ICDs), respectively. According to some embodiments, the first cytokine receptor ICD, the second cytokine receptor ICD, or both, comprises an IL-2Rβ ICD, IL-3Rα ICD, IL-4R ICD, IL-5Rα ICD, IL-6Rα ICD, IL-7R ICD, IL-9R ICD, IL-10Rα ICD, IL-10Rβ ICD, IL-11Rα ICD, IL-12Rβ1 ICD, IL-12Rβ2 ICD, IL-13Rα1 ICD, IL-13Rα2 ICD, IL-20Rα ICD, IL-20Rβ, IL-21R ICD, IL-22Rα1, IL-22Rα2, IL-23R ICD, IL-27Rα ICD, IL-28RA ICD, IL-31Rα ICD, TGFβR1 ICD, TGFβR2 ICD, TGFβR3 ICD, IFNAR1 ICD, IFNAR2 ICD, IFNGR1 ICD, IFNGR2 ICD, IFNLR ICD, CSF2RB ICD, gp130 ICD, CD9 ICD, OSMR ICD, CSF-1R ICD, or any combination thereof.

In certain embodiments, the first cytokine receptor ICD is an IL-22Rα1 ICD, and the second cytokine receptor ICD is an IL-10Rβ ICD. Such a CCR finds use, e.g., when it is desired for a cell to exhibit constitutive or regulatable IL-22 signaling in the absence of IL-22. According to some embodiments, the first cytokine receptor ICD is an IL-23R ICD and the second cytokine receptor ICD is an IL-12Rβ1 ICD. Such a CCR finds use, e.g., when it is desired for a cell to exhibit constitutive or regulatable IL-23 signaling in the absence of IL-23.

According to some embodiments, the first cytokine receptor ICD comprises an ICD from a common cytokine receptor gamma-chain family. γc serves as a shared signaling receptor for IL-2, IL-4, IL-7, IL-9, IL-15, and IL-21. In certain embodiments, the ICD from a common cytokine receptor gamma-chain family is an IL-2Rβ ICD, IL-4R ICD, IL-7R ICD, IL-9R ICD, or an IL-21R ICD. When the first cytokine receptor ICD comprises an ICD from a common cytokine receptor gamma-chain family, in some embodiments, the second cytokine receptor ICD is a common gamma chain (γc) ICD.

In certain embodiments, the first subunit, the second subunit, or both, comprises a linker sequence between domains of the subunit. Such a subunit may include one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long. Non-limiting examples of linkers that may be incorporated between domains of the first and/or second subunit include serine-glycine linkers, disulfide linkers (e.g., to enhance association between the first and second subunits), and the like, and include any of the linker sequences provided in Table 1 below.

According to some embodiments, the first subunit, the second subunit, or both, comprise a transition domain disposed between the transmembrane domain and an ICD. As will be appreciated with the benefit of the present disclosure, such a transition domain may be incorporated into the first and/or second subunits to enhance expression of the CCR as compared to the subunit in the absence of the transition domain, as demonstrated in the Experimental section below. For example, in some embodiments, a subunit comprising a γc ICD may be engineered such that a transition domain is disposed between the transmembrane domain and the γc ICD. Suitable transition domains include, but are not limited to, transition domains comprising a juxtamembrane (JM) region of a cell surface receptor. In some embodiments, a JM region of CD8a or IL-15Rα is employed as a transition domain between a transmembrane domain and an ICD, e.g., a γc ICD. A non-limiting example of a CD8a transition domain (sometimes referred to herein as a transition peptide) is a domain comprising the amino acid sequence NHRNRRRVCKCPRPVV (SEQ ID NO:54). A non-limiting example of an IL-15Ra transition domain is a domain comprising the amino acid sequence KSRQTPP (SEQ ID NO:55). Variants of such domains are also provided by the present disclosure.

In certain embodiments, the first subunit and the second subunit are encoded by a single nucleic acid. When the first subunit and the second subunit are encoded by a single nucleic acid, the nucleic acid may be configured to allow for polycistronic expression of the first subunit and the second subunit. That is, two or more (e.g., each) of the proteins encoded by a single nucleic acid may be expressed as separate proteins from the same promoter. In certain embodiments, the single nucleic acid includes a ribosome skipping site to allow for polycistronic expression of two or more (e.g., each) of the protein-encoding regions. A non-limiting example of a suitable ribosome skipping site which may be incorporated into such a single nucleic acid is the P2A ribosome skipping site from porcine teschovirus. According to other embodiments, the first subunit is encoded by a first nucleic acid and the second subunit is encoded by a second nucleic acid.

According to some embodiments, the first subunit, the second subunit, or both, comprises a protease cleavage site disposed between the transmembrane domain and the ICD. The term “cleavage site” refers to the bond (e.g., a scissile bond) cleaved by an agent, e.g., a protease. A cleavage site for a protease includes the specific amino acid sequence recognized by the protease during proteolytic cleavage and may include surrounding amino acids (e.g., from one to six amino acids) on either side of the scissile bond, which bind to the active site of the protease and are needed for recognition as a substrate. In some embodiments, the cleavage site is provided as a cleavable linker, where “cleavable linker” refers to a linker including the protease cleavage site. A cleavable linker is typically cleavable under physiological conditions. According to some embodiments, the protease cleavage site is a viral protease cleavage site. Non-limiting examples of viral protease cleavage sites include cleavage sites for potyviral family proteases. Potyviral family proteases of interest include Tobacco Etch Virus (TEV) protease, plum pox virus protease (PPVp), soybean mosaic virus protease (SbMVp), sunflower mild mosaic virus protease (SuMMVp), tobacco vein mottling virus protease (TVMVp), and West Nile virus protease (WNVp). In certain embodiments, the viral protease cleavage site is a TEV protease cleavage site. The amino acid sequence of an example TEV protease cleavage site is ENLYFQS (SEQ ID NO:58). The amino acid sequence of an example TEV protease is the following:

(SEQ ID NO: 59)
GESLFKGPRDYNPISSTICHLTNESDGHTTSLYGIGFGPFIITNK
HLFRRNNGTLLVQSLHGVFKVKNTTTLQQHLIDGRDMIIIRMPKD
FPPFPQKLKFREPQREERICLVTTNFQTKSMSSMVSDTSCTFPSS
DGIFWKHWIQTKDGQCGSPLVSTRDGFIVGIHSASNFTNTNNYFT
SVPKNFMELLTNQEAQQWVSGWRLNADSVLWGGHKVFMVKPEEPF
QPVKEATQLMN

In some embodiments, the protease is a TEV protease comprising the amino acid sequence set forth above, or is a functional (proteolytic) variant thereof having 70% or greater, 75% or greater, 80% or greater, 85% or greater, 90% or greater, 95% or greater, or 99% or greater amino acid sequence identity to such a sequence, and/or a functional (proteolytic) fragment thereof such as a fragment having a length of from 100 to 125, 125 to 150, 150 to 175, 175 to 200, 200 to 225, or from 225 to 235 amino acids.

According to some embodiments, the viral protease cleavage site is for an HCV protease. In certain embodiments, the viral protease cleavage site is for a viral protease derived from HCV nonstructural protein 3 (NS3). NS3 consists of an N-terminal serine protease domain and a C-terminal helicase domain. By “derived from HCV NS3” is meant the protease is the serine protease domain of HCV NS3 or a proteolytically active variant thereof capable of cleaving a cleavage site for the serine protease domain of HCV NS3. The protease domain of NS3 forms a heterodimer with the HCV nonstructural protein 4A (NS4A), which activates proteolytic activity. A protease derived from HCV NS3 may include the entire NS3 protein or a proteolytically active fragment thereof, and may further include a cofactor polypeptide, such as a cofactor polypeptide derived from HCV nonstructural protein 4A (NS4A), e.g., an activating NS4A region. NS3 protease is highly selective and can be inhibited by a number of non-toxic, cell-permeable drugs, which are currently available for use in humans. NS3 protease inhibitors that may be employed include, but are not limited to, asunaprevir (ASV), danoprevir (DPV), simeprevir (SPV), grazoprevir (GPV), glecaprevir, voxilaprevir, and any combination thereof. Non-limiting examples of proteases derived from HCV NS3 are provided below.

Example Proteases Derived from HCV NS3

(SEQ ID NO: 60)
APITAYAQQTRGLLGCIITSLTGRDKNQVEGEVQIVSTATQTFLA
TCINGVCWAVYHGAGTRTIASPKGPVIQMYTNVDQDLVGWPAPQG
SRSLTPCTCGSSDLYLVTRHADVIPVRRRGDSRGSLLSPRPISYL
KGSSGGPLLCPAGHAVGLFRAAVCTRGVAKAVDFIPVENLETTMR
SPVFTD
(SEQ ID NO: 61)
APITAYAQQTRGLLGCIITSLTGRDKNQVEGEVQIMSTATQTFLA
TCINGVCWTVYHGAGTRTIASPKGPVIQMYTNVDQDLVGWPAPQG
SRSLTPCTCGSSDLYLVTRHADVIPVRRRGDGRGSLLSPRPISYL
KGSSGGPLLCPAGHAVGLFRAAVCTRGVAKAVDFIPVENLETTMR
SPVFTD
(SEQ ID NO: 62)
APITAYAQQTRGLLGCIITSLTGRDKNQVEGEVQIVSTATQTFLA
TCINGVCWTVYHGAGTRTIASPKGPVIQMYTNVDQDLVGWPAPQG
SRSLTPCTCGSSDLYLVTRHADVIPVRRRGDSRGSLLSPRPISYL
KGSSGGPLLCPAGHAVGLFRAAVCTRGVAKAVDFIPVENLETTMR
SPVFTD

In some embodiments, the protease comprises one of the sequences set forth above, or is a functional (proteolytic) variant thereof having 70% or greater, 75% or greater, 80% or greater, 85% or greater, 90% or greater, 95% or greater, or 99% or greater amino acid sequence identity to one of such sequences, and/or a functional (proteolytic) fragment thereof such as a fragment having a length of from 100 to 185, 120 to 185, 140 to 185, 160 to 185, 170 to 185, from 180 to 185, from 182 to 185, or from 184 to 185 amino acids.

An NS3 protease cleavage site may include the four junctions between nonstructural (NS) proteins of the HCV polyprotein normally cleaved by the NS3 protease during HCV infection, including the NS3/NS4A, NS4A/NS4B, NS4B/NS5A, and NS5A/NS5B junction cleavage sites. For a description of NS3 protease and representative sequences of its cleavage sites for various strains of HCV, see, e.g., Hepatitis C Viruses: Genomes and Molecular Biology (S. L. Tan ed., Taylor & Francis, 2006), Chapter 6, pp. 163-206; the disclosure of which is incorporated herein by reference in its entirety.

In some embodiments, the protease is derived from HCV NS3 and engineered to include one or more amino acid substitutions relative to an HCV NS3 protease amino acid sequence set forth above. For example, the protease may include a substitution at the position corresponding to position 54 of the amino acid sequence APITAYAQQTRGLLGCIITSLTGRDKNQVEGEVQIVSTATQTFLATCINGVCWAVYHGAGTRTIA SPKGPVIQMYTNVDQDLVGWPAPQGSRSLTPCTCGSSDLYLVTRHADVIPVRRRGDSRGSLL SPRPISYLKGSSGGPLLCPAGHAVGLFRAAVCTRGVAKAVDFIPVENLETTMRSPVFTD (SEQ ID NO: 60). In some embodiments, such a substitution is a threonine to alanine substitution.

NS3 nucleic acid and protein sequences may be derived from HCV, including any isolate of HCV having any genotype (e.g., genotypes 1-7) or subtype. A number of NS3 nucleic acid and protein sequences are known and described, e.g., in U.S. Ser. No. 15/737,712, the disclosure of which is incorporated herein by reference in their entirety for all purposes. Additional representative NS3 sequences are listed in the National Center for Biotechnology Information (NCBI) database. See, for example, NCBI entries: Accession Nos. YP_001491553, YP_001469631, YP_001469632, NP_803144, NP_671491, YP_001469634, YP_001469630, YP_001469633, ADA68311, ADA68307, AFP99000, AFP98987, ADA68322, AFP99033, ADA68330, AFP99056, AFP99041, CBF60982, CBF60817, AHH29575, AIZ00747, AIZ00744, ABI36969, ABN05226, KF516075, KF516074, KF516056, AB826684, AB826683, JX171009, JX171008, JX171000, EU847455, EF154714, GU085487, JX171065, and JX171063; all of which sequences are herein incorporated by reference. Any of these sequences or functional variants thereof having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, or 99% or greater amino acid sequence identity to any one of these sequences, or proteolytic fragments thereof, may be employed.

NS4A nucleic acid and protein sequences may be derived from HCV, including any isolate of HCV having any genotype (e.g., seven genotypes 1-7) or subtype. A number of NS4A nucleic acid and protein sequences are known. Representative NS4A sequences are listed in the National Center for Biotechnology Information (NCBI) database. See, for example, NCBI entries: Accession Nos. NP_751925, YP_001491554, GU945462, HQ822054, FJ932208, FJ932207, FJ932205, and FJ932199; all of which sequences (as entered by the date of filing of this application) are herein incorporated by reference. Any of these sequences or functional variants thereof having 70% or greater, 75% or greater, 80% or greater, 85% or greater, 90% or greater, 95% or greater, or 99% or greater amino acid sequence identity to any one of these sequences, or proteolytic fragments thereof, may be employed.

HCV polyprotein nucleic acid and protein sequences may be derived from HCV, including any isolate of HCV having any genotype (e.g., genotypes 1-7) or subtype. A number of HCV polyprotein nucleic acid and protein sequences are known. Representative HCV polyprotein sequences are listed in the National Center for Biotechnology Information (NCBI) database. See, for example, NCBI entries: Accession Nos. YP_001469631, NP_671491, YP_001469633, YP_001469630, YP_001469634, YP_001469632, NC_009824, NC_004102, NC_009825, NC_009827, NC_009823, NC_009826, and EF108306; all of which sequences (as entered by the date of filing of this application) are herein incorporated by reference. Any of these sequences or functional variants thereof having 70% or greater, 75% or greater, 80% or greater, 85% or greater, 90% or greater, 95% or greater, or 99% or greater amino acid sequence identity to any one of these sequences, or proteolytic fragments thereof, may be employed.

According to some embodiments, when the protease cleavage site is a viral protease cleavage site, the viral protease cleavage site is a human immunodeficiency virus (HIV) protease cleavage site. In certain embodiments, when the protease cleavage site is a viral protease cleavage site, the viral protease cleavage site is a SARS-COV-2 protease cleavage site.

In certain embodiments, the protease cleavage site is a human protease cleavage site. Non-limiting examples of human protease cleavage sites include cleavages sites for a human kallikrein (KLK) protease, human enterokinase protease, human thrombin, a human matrix metalloprotease (MMP), human urokinase-type plasminogen activator receptor (uPAR), human plasmin, or human cathepsin. According to some embodiments, the protease cleavage site is a cleavage site for a human kallikrein (KLK) protease, non-limiting examples of which include human KLK3 (UniProtKB—Q546G3), human KLK4 (UniProtKB—Q9Y5K2), human KLK6 (UniProtKB—Q92876), human KLK8 (UniProtKB—O60259), human KLK11 (UniProtKB—Q9UBX7), human KLK13 (UniProtKB—Q9UKR3), human KLK14 (UniProtKB—Q9P0G3), and human KLK15 (UniProtKB—Q9H2R5). Any of these sequences or functional variants thereof having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, or 99% or greater amino acid sequence identity to any one of these sequences, or proteolytic fragments thereof, may be employed.

In certain embodiments, the protease cleavage site is a protease cleavage site for a human protease selected from acrosin (ACR), AGBL carboxypeptidase 1 (AGBL1), AGBL carboxypeptidase 2 (AGBL2), AGBL carboxypeptidase 3 (AGBL3), AGBL carboxypeptidase 4 (AGBL4), AGBL carboxypeptidase 5 (AGBL5), ATP/GTP binding carboxypeptidase 1 (AGTPBP1), asparaginase and isoaspartyl peptidase 1 (ASRGL1), astacin like metalloendopeptidase (ASTL), ATP23 metallopeptidase and ATP synthase assembly factor homolog (ATP23), ataxin 3 (ATXN3), ataxin 3 like (ATXN3L), azurocidin 1 (AZU1), beta-secretase 1 (BACE1), beta-secretase 2 (BACE2), bone morphogenetic protein 1 (BMP1), BRCA1/BRCA2-containing complex subunit 3 (BRCC3), calpain 14 (CAPN14), calpain 3 (CAPN3), caspase recruitment domain family member 8 (CARD8), caspase 4 (CASP4), chymotrypsin like elastase 1 (CELA1), chymotrypsin like elastase 2A (CELA2A), chymotrypsin like elastase 2B (CELA2B), chymotrypsin like elastase 3A (CELA3A), chymotrypsin like elastase 3B (CELA3B), CUGBP Elav-like family member 3 (CELF3), CUGBP Elav-like family member 4 (CELF4), CUGBP Elav-like family member 5 (CELF5), CUGBP Elav-like family member 6 (CELF6), cell growth regulator with EF-hand domain 1 (CGREF1), charged multivesicular body protein 3 (CHMP3), CLN5 intracellular trafficking protein (CLN5), chymase 1 (CMA1), collectin subfamily member 11 (COLEC11), COP9 signalosome subunit 5 (COPS5), corin, serine peptidase (CORIN), carboxypeptidase A4 (CPA4), carboxypeptidase vitellogenic like (CPVL), cystatin SN (CST1), cystatin 11 (CST11), cystatin C (CST3), cystatin S (CST4), cystatin D (CST5), cystatin E/M (CST6), cystatin 8 (CST8), cystatin 9 (CST9), cystatin like 1 (CSTL1), chymotrypsinogen B2 (CTRB2), chymotrypsin like (CTRL), cathepsin L (CTSL), DNA damage inducible 1 homolog 2 (DDI2), DAP3 binding cell death enhancer 1 (DELE1), adipsin (DF), dickkopf WNT signaling pathway inhibitor 2 (DKK2), dickkopf WNT signaling pathway inhibitor 4 (DKK4), dipeptidase 1 (DPEP1), dipeptidyl peptidase 3 (DPP3), dipeptidyl peptidase 9 (DPP9), FAM111 trypsin like peptidase A (FAM111A), ficolin 1 (FCN1), ficolin 2 (FCN2), ficolin 3 (FCN3), G3BP stress granule assembly factor 1 (G3BP1), hepsin (HPN), HtrA serine peptidase 1 (HTRA1), insulin degrading enzyme (IDE), inner mitochondrial membrane peptidase subunit 2 (IMMP2L), jumonji domain containing 7 (JMJD7), Josephin domain containing 2 (JOSD2), kallikrein 1 (KLK1), kallikrein related peptidase 10 (KLK10), kallikrein related peptidase 11 (KLK11), kallikrein related peptidase 12 (KLK12), kallikrein related peptidase 13 (KLK13), kallikrein related peptidase 14 (KLK14), kallikrein related peptidase 15 (KLK15), kallikrein related peptidase 2 (KLK2), kallikrein related peptidase 3 (KLK3), kallikrein related peptidase 4 (KLK4), kallikrein related peptidase 5 (KLK5), kallikrein related peptidase 6 (KLK6), kallikrein related peptidase 7 (KLK7), kallikrein related peptidase 8 (KLK8), kallikrein related peptidase 9 (KLK9), kallikrein pseudogene 1 (KLKP1), lipocalin 2 (LCN2), legumain (LGMN), leishmanolysin like peptidase (LMLN), MAS1 proto-oncogene like, G protein-coupled receptor (MAS1L), MBL associated serine protease 1 (MASP1), MBL associated serine protease 2 (MASP2), mannose binding lectin 2 (MBL2), matrix metallopeptidase 10 (MMP10), matrix metallopeptidase 11 (MMP11), matrix metallopeptidase 13 (MMP13), matrix metallopeptidase 16 (MMP16), matrix metallopeptidase 2 (MMP2), napsin A aspartic peptidase (NAPSA), neurolysin (NLN), NLR family CARD domain containing 4 (NLRC4), NLR family pyrin domain containing 1 (NLRP1), aminopeptidase puromycin sensitive (NPEPPS), opiorphin prepropeptide (OPRPN), OTU deubiquitinase, ubiquitin aldehyde binding 2 (OTUB2), poly (ADP-ribose) polymerase family member 9 (PARP9), proprotein convertase subtilisin/kexin type 1 (PCSK1), proprotein convertase subtilisin/kexin type 1 inhibitor (PCSK1N), proprotein convertase subtilisin/kexin type 2 (PCSK2), proprotein convertase subtilisin/kexin type 4 (PCSK4), proprotein convertase subtilisin/kexin type 5 (PCSK5), proprotein convertase subtilisin/kexin type 6 (PCSK6), proprotein convertase subtilisin/kexin type 7 (PCSK7), proprotein convertase subtilisin/kexin type 9 (PCSK9), platelet derived growth factor C (PDGFC), pepsinogen A3 (PGA3), pepsinogen A4 (PGA4), pepsinogen A5 (PGA5), pyroglutamyl-peptidase I like (PGPEP1L), PTEN induced kinase 1 (PINK1), prolyl endopeptidase like (PREPL), parkin RBR E3 ubiquitin protein ligase (PRKN), serine protease gene group (PRSS), serine protease 2 (PRSS2), serine protease 21 (PRSS21), serine protease 22 (PRSS22), serine protease 23 (PRSS23), serine protease 27 (PRSS27), serine protease 33 (PRSS33), serine protease 46, pseudogene (PRSS46P), serine protease 55 (PRSS55), serine protease 8 (PRSS8), proteinase 3 (PRTN3), presenilin 2 (PSEN2), PYD and CARD domain containing (PYCARD), retinoic acid receptor responder 1 (RARRES1), ring finger and FYVE like domain containing E3 ubiquitin protein ligase (RFFL), rhomboid like 2 (RHBDL2), SEC11 homolog A, signal peptidase complex subunit (SEC11A), SEC11 homolog B, signal peptidase complex subunit (SEC11B), SEC11 homolog C, signal peptidase complex subunit (SEC11BC), SUMO peptidase family member, NEDD8 specific (SENP8), SET nuclear proto-oncogene (SET), synaptosome associated protein 25 (SNAP25), secreted phosphoprotein 2 (SPP2), small proline rich protein 3 (SPRR3), spleen associated tyrosine kinase (SYK), transcription factor EB (TFEB), transglutaminase 2 (TGM2), toll like receptor adaptor molecule 1 (TICAM1), tubulointerstitial nephritis antigen like 1 (TINAGL1), transmembrane serine protease 11D (TMPRSS11D), transmembrane serine protease 11E (TMPRSS11E), transmembrane serine protease 4 (TMPRSS4), transmembrane serine protease 5 (TMPRSS5), transmembrane serine protease 6 (TMPRSS6), transmembrane serine protease 7 (TMPRSS7), TNF receptor superfamily member 10a (TNFRSF10A), tryptase alpha/beta 1 (TPSAB1), tryptase beta 2 (TPSB2), tryptase delta 1 (TPSD1), tryptase gamma 1 (TPSG1), tryptase pseudogene 2 (TPSP2), tyrosylprotein sulfotransferase 1 (TPST1), tyrosylprotein sulfotransferase 2 (TPST2), tyrosylprotein sulfotransferase 2 pseudogene 1 (TPST2P1), thyrotropin releasing hormone degrading enzyme (TRHDE), thyroid hormone receptor interactor 4 (TRIP4), ubiquitin C-terminal hydrolase L1 (UCHL1), ubiquitin specific peptidase 27 X-linked (USP27X), vasohibin 2 (VASH2), valosin containing protein (VCP), and WAP four-disulfide core domain 1 (WFDC1).

In some embodiments, the protease is highly selective for the cleavage site. Additionally, the protease activity may be capable of inhibition by known small molecule inhibitors that are cell-permeable and not toxic to the cell or individual under study or treatment. For a discussion of proteases, see, e.g., V. Y. H. Hook, Proteolytic and cellular mechanisms in prohormone and proprotein processing, RG Landes Company, Austin, Tex., USA (1998); N. M. Hooper et al., Biochem. J. 321:265-279 (1997); Z. Werb, Cell 91:439-442 (1997); T. G. Wolfsberg et al., J. Cell Biol. 131:275-278 (1995); T. Berg et al., Biochem. J. 307:313-326 (1995); M. J. Smyth and J. A. Trapani, Immunology Today 16:202-206 (1995); R. V. Talanian et al., J. Biol. Chem. 272:9677-9682 (1997); and N. A. Thornberry et al., J. Biol. Chem. 272:17907-17911 (1997), the disclosures of which are incorporated herein by reference in their entireties for all purposes. In some embodiments, the protease employed is a sequence-specific non-human protease for which FDA-approved pharmacological inhibitors are available.

Accordingly, in some embodiments, the one or more nucleic acids further encode a protease, where the protease cleavage site is a cleavage site for the protease. In one configuration (sometimes referred to herein as a “trans” configuration in which the protease is expressed as a separate polypeptide with respect to the first and second subunits), the one or more nucleic acids encode a fusion protein comprising the protease and a transmembrane domain. In other instances, a protease may be expressed as part of a fusion protein with the first subunit, the second subunit, or both, sometimes referred to herein as a “cis” configuration.

Proteolytic activity of the protease may be regulatable. In some embodiments, proteolytic activity of the protease is regulatable via a cell-permeable small molecule. For example, such a cell-permeable small molecule may be an inhibitor of the protease. Such inhibitors are known and available. When the protease is derived from HCV NS3, in some instances, the inhibitor of the protease is selected from asunaprevir (ASV), danoprevir (DPV), simeprevir (SPV), grazoprevir (GPV), glecaprevir, voxilaprevir, and any combination thereof. When the protease is an HIV protease, in some instances, the inhibitor of the protease is selected from atazanavir (Reyataz), darunavir (Prezista), fosamprenavir (Lexiva), indinavir (Crixivan), lopinavir/ritonavir (Kaletra), nelfinavir (Viracept), ritonavir (Norvir), saquinavir (Invirase), tipranavir (Aptivus), atazanavir/cobicistat (Evotaz), darunavir/cobicistat (Prezcobix), and any combination thereof. When the protease is a SARS-COV-2 protease, in some instances, the inhibitor of the protease is nirmatrelvir (Paxlovid). When the protease is a human renin protease, in some instances, the inhibitor of the protease is aliskiren.

The first and/or second subunits may include any additional domains and functionalities as desired. For example, in certain embodiments, the first subunit, the second subunit, or both, comprises an extracellular protein tag. Such tags find use, e.g., in assessing cells for cell surface expression of a CCR. In some instances, the extracellular protein tag is an N-terminal protein tag. Non-limiting examples tags that may be incorporated into the first and/or second subunit include a FLAG tag, an HA tag, or the like. Example amino acid sequences of such tags are provided in Table 1 below.

In certain embodiments, the first subunit and the second subunit are engineered to each comprise an extracellular cysteine residue, wherein disulfide bonding between the extracellular cysteine residue of the first subunit and the extracellular cysteine residue of the second subunit stabilizes the association of the first subunit and the second subunit when expressed on the surface of a cell. For example, a disulfide linker sequence (e.g., CGG) may be incorporated into the extracellular domain of each of the first and second subunits to stabilize their association.

Aspects of the present disclosure further include chimeric cytokine receptors (CCRs) encoded by the one or more nucleic acids of the present disclosure.

The amino acid sequences of exemplary CCR components are provided in Table 1 below. For each sequence, the domains as ordered from N- to C-terminus are listed in the left column. The sequence in the right column indicates the domains by alternating underlining.

TABLE 1
Amino Acid Sequences of Exemplary CCR Components

| | | SEQ ID |
|---|---|---|
| Description | Sequence | NO: |
| # 223 FLAG Tag.RR zipper. | MLLLVTSLLLCELPHPAFLLIPDYKDDDDKCGGK | 1 |
| CD8α H/TM. IL2Rβ ICD | GGGLEIRAAFLRRRNTALRTRVAELRQRVQRAR | |
| GM-CSF leader sequence | NRVSQYRTRYGPLTTTPAPRPPTPAPTIASQPLS | |
| FLAG Tag | LRPEACRPAAGGAVHTRGLDFACDIYIWAPLAG | |
| Disulfide linker | TCGVLLLSLVITLYCNCRNTGPWLKKVLKCNTPD | |
| RR leucine zipper | PSKFFSQLSSEHGGDVQKWLSSPFPSSSFSPG | |
| CD8α hinge | GLAPEISPLEVLERDKVTQLLLQQDKVPEPASLS | |
| CD8α transmembrane | SNHSLTSCFTNQGYFFFHLPDALEIEACQVYFTY | |
| domain | DPYSEEDPDEGVAGAPTGSSPQPLQPLSGEDD | |
| IL2Rβ intracellular domain | AYCTFPSRDDLLLFSPSLLGGPSPPSTAPGGSG | |
| | AGEERMPPSLQERVPRDWDPQPLGPPTPGVPD | |
| | LVDFQPPPELVLREAGEEVPDAGPREGVSFPWS | |
| | RPPGQGEFRALNARLPLNTDAYLSLQELQGQDP | |
| | THLV | |
| # 225 FLAG Tag.RR zipper. | MLLLVTSLLLCELPHPAFLLIPDYKDDDDKCGGK | 2 |
| CD8α H/TM. IL 15Ra ICD | GGGLEIRAAFLRRRNTALRTRVAELRQRVQRAR | |
| GM-CSF leader sequence | NRVSQYRTRYGPLTTTPAPRPPTPAPTIASQPLS | |
| FLAG Tag | LRPEACRPAAGGAVHTRGLDFACDIYIWAPLAG | |
| Disulfide linker | TCGVLLLSLVITLYCKSRQTPPLASVEMEAMEAL | |
| RR leucine zipper | PVTWGTSSRDEDLENCSHHL | |
| CD8α hinge | | |
| CD8α transmembrane | | |
| domain | | |
| IL15Rα intracellular | | |
| domain | | |
| # 226 FLAG Tag.RR zipper. | MLLLVTSLLLCELPHPAFLLIPDYKDDDDKCGGK | 3 |
| CD8α H/TM. IL21R ICD | GGGLEIRAAFLRRRNTALRTRVAELRQRVQRAR | |
| GM-CSF leader sequence | NRVSQYRTRYGPLTTTPAPRPPTPAPTIASQPLS | |
| FLAG Tag | LRPEACRPAAGGAVHTRGLDFACDIYIWAPLAG | |
| Disulfide linker | TCGVLLLSLVITLYCSLKTHPLWRLWKKIWAVPS | |
| RR leucine zipper | PERFFMPLYKGCSGDFKKWVGAPFTGSSLELG | |
| CD8α hinge | PWSPEVPSTLEVYSCHPPRSPAKRLQLTELQEP | |
| CD8α transmembrane | AELVESDGVPKPSFWPTAQNSGGSAYSEERDR | |
| domain | PYGLVSIDTVTVLDAEGPCTWPCSCEDDGYPAL | |
| IL21R intracellular domain | DLDAGLEPSPGLEDPLLDAGTTVLSCGCVSAGS | |
| | PGLGGPLGSLLDRLKPPLADGEDWAGGLPWGG | |
| | RSPGGVSESEAGSPLAGLDMDTFDSGFVGSDC | |
| | SSPVECDFTSPGDEGPPRSYLRQWVVIPPPLSS | |
| | PGPQAS | |
| 227 HA Tag.EE zipper.CD8α | MLLLVTSLLLCELPHPAFLLIPYPYDVPDYACGGSS | 4 |
| H/TM.γ-chain ICD | GLEIEAAFLEQENTALETEVAELEQEVQRLENEVSQ | |
| GM-CSF leader sequence | YETRYGPLGGGKTTTPAPRPPTPAPTIASQPLSLR | |
| HA Tag | PEACRPAAGGAVHTRGLDFACDIYIWAPLAGTCGV | |
| Disulfide linker | LLLSLVITLYCERTMPRIPTLKNLEDLVTEYHGNFSA | |
| EE leucine zipper | WSGVSKGLAESLQPDYSERLCLVSEIPPKGGALGE | |
| CD8α hinge | GPGASPCNQHSPYWAPPCYTLKPET | |
| CD8α transmembrane | | |
| domain | | |
| γ-chain ICD | | |
| 228 HA Tag.EE zipper.CD8α | MLLLVTSLLLCELPHPAFLLIPYPYDVPDYACGGSS | 5 |
| H/TM.linker (GGSGS). γ-chain ICD | GLEIEAAFLEQENTALETEVAELEQEVQRLENEVSQ | |
| GM-CSF leader sequence | YETRYGPLGGGKTTTPAPRPPTPAPTIASQPLSLR | |
| HA Tag | PEACRPAAGGAVHTRGLDFACDIYIWAPLAGTCGV | |
| Disulfide linker | LLLSLVITLYCGGSGSERTMPRIPTLKNLEDLVTEYH | |
| EE leucine zipper | GNFSAWSGVSKGLAESLQPDYSERLCLVSEIPPKG | |
| CD8α hinge | GALGEGPGASPCNQHSPYWAPPCYTLKPET | |
| CD8α transmembrane | | |
| domain | | |
| Linker (GGSGS) | | |
| γ-chain ICD | | |
| 249 HA Tag.EE zipper.CD8α | MLLLVTSLLLCELPHPAFLLIPYPYDVPDYACGGSS | 6 |
| H/TM.CD8α transition peptide.γ- | GLEIEAAFLEQENTALETEVAELEQEVQRLENEVSQ | |
| chain ICD | YETRYGPLGGGKTTTPAPRPPTPAPTIASQPLSLR | |
| GM-CSF leader sequence | PEACRPAAGGAVHTRGLDFACDIYIWAPLAGTCGV | |
| HA Tag | LLLSLVITLYCNHRNRRRVCKCPRPVVERTMPRIPT | |
| Disulfide linker | LKNLEDLVTEYHGNFSAWSGVSKGLAESLQPDYS | |
| EE leucine zipper | ERLCLVSEIPPKGGALGEGPGASPCNQHSPYWAP | |
| CD8α hinge | PCYTLKPET | |
| CD8α transmembrane | | |
| domain | | |
| CD8α transition peptide | | |
| γ-chain ICD | | |
| 250 HA Tag.EE zipper.CD8α | MLLLVTSLLLCELPHPAFLLIPYPYDVPDYACGGSS | 7 |
| H/TM.IL 15Ra transition | GLEIEAAFLEQENTALETEVAELEQEVQRLENEVSQ | |
| peptide. γ-chain ICD | YETRYGPLGGGKTTTPAPRPPTPAPTIASQPLSLR | |
| GM-CSF leader sequence | PEACRPAAGGAVHTRGLDFACDIYIWAPLAGTCGV | |
| HA Tag | LLLSLVITLYCKSRQTPPERTMPRIPTLKNLEDLVTE | |
| Disulfide linker | YHGNFSAWSGVSKGLAESLQPDYSERLCLVSEIPP | |
| EE leucine zipper | KGGALGEGPGASPCNQHSPYWAPPCYTLKPET | |
| CD8α hinge | | |
| CD8α transmembrane | | |
| domain | | |
| IL15Rα transition peptide | | |
| γ-chain ICD | | |
| # 364 Bicistronic constitutive IL2 | MLLLVTSLLLCELPHPAFLLIPDYKDDDDKCGGKGG | 8 |
| CCR (FLAG Tag.RR zipper. CD8α | GLEIRAAFLRRRNTALRTRVAELRQRVQRARNRVS | |
| H/TM. IL2Rβ ICD.P2A.HA Tag.EE | QYRTRYGPLTTTPAPRPPTPAPTIASQPLSLRPEAC | |
| zipper.CD8α H/TM.IL15Rα | RPAAGGAVHTRGLDFACDIYIWAPLAGTCGVLLLSL | |
| transition peptide.γ-chain ICD) | VITLYCNCRNTGPWLKKVLKCNTPDPSKFFSQLSS | |
| GM-CSF leader sequence | EHGGDVQKWLSSPFPSSSFSPGGLAPEISPLEVLE | |
| FLAG | RDKVTQLLLQQDKVPEPASLSSNHSLTSCFTNQGY | |
| Tag | FFFHLPDALEIEACQVYFTYDPYSEEDPDEGVAGA | |
| Disulfide linker | PTGSSPQPLQPLSGEDDAYCTFPSRDDLLLFSPSL | |
| RR leucine zipper | LGGPSPPSTAPGGSGAGEERMPPSLQERVPRDW | |
| CD8α hinge | DPQPLGPPTPGVPDLVDFQPPPELVLREAGEEVPD | |
| CD8α transmembrane | AGPREGVSFPWSRPPGQGEFRALNARLPLNTDAY | |
| domain | LSLQELQGQDPTHLVSIGATNFSLLKQAGDVEENP | |
| IL2Rβ intracellular domain | GPGSTMLLLVTSLLLCELPHPAFLLIPYPYDVPDYA | |
| Linker | CGGSSGLEIEAAFLEQENTALETEVAELEQEVQRL | |
| P2A | ENEVSQYETRYGPLGGGKTTTPAPRPPTPAPTIAS | |
| Linker | QPLSLRPEACRPAAGGAVHTRGLDFACDIYIWAPL | |
| GM-CSF leader sequence | AGTCGVLLLSLVITLYCKSRQTPPERTMPRIPTLKN | |
| HA Tag | LEDLVTEYHGNFSAWSGVSKGLAESLQPDYSERL | |
| Disulfide linker | CLVSEIPPKGGALGEGPGASPCNQHSPYWAPPCY | |
| EE leucine zipper | TLKPET | |
| hinge | | |
| CD8α transmembrane | | |
| domain | | |
| IL15Rα transition peptide | | |
| γ-chain ICD | | |
| # 476 Bicistronic IL2 CCR with | MLLLVTSLLLCELPHPAFLLIPDYKDDDDKCGGKGG | 9 |
| 4a4b cleavage site (FLAG Tag.RR | GLEIRAAFLRRRNTALRTRVAELRQRVQRARNRVS | |
| zipper. CD8α H/TM. IL2Rβ | QYRTRYGPLTTTPAPRPPTPAPTIASQPLSLRPEAC | |
| ICD.P2A.HA Tag. EE zipper.CD8α | RPAAGGAVHTRGLDFACDIYIWAPLAGTCGVLLLSL | |
| H/TM.IL 15Ra transition peptide. | VITLYCNCRNTGPWLKKVLKCNTPDPSKFFSQLSS | |
| HCV 4a4b cleavage site.γ-chain | EHGGDVQKWLSSPFPSSSFSPGGLAPEISPLEVLE | |
| ICD) | RDKVTQLLLQQDKVPEPASLSSNHSLTSCFTNQGY | |
| GM-CSF leader sequence | FFFHLPDALEIEACQVYFTYDPYSEEDPDEGVAGA | |
| FLAG Tag | PTGSSPQPLQPLSGEDDAYCTFPSRDDLLLFSPSL | |
| Disulfide linker | LGGPSPPSTAPGGSGAGEERMPPSLQERVPRDW | |
| RR leucine zipper | DPQPLGPPTPGVPDLVDFQPPPELVLREAGEEVPD | |
| CD8α | AGPREGVSFPWSRPPGQGEFRALNARLPLNTDAY | |
| hinge | LSLQELQGQDPTHLVSIGATNFSLLKQAGDVEENP | |
| | GPGSTMLLLVTSLLLCELPHPAFLLIPYPYDVPDYA | |
| CD8α transmembrane | CGGSSGLEIEAAFLEQENTALETEVAELEQEVQRL | |
| domain | ENEVSQYETRYGPLGGGKTTTPAPRPPTPAPTIAS | |
| IL2Rβ intracellular domain | QPLSLRPEACRPAAGGAVHTRGLDFACDIYIWAPL | |
| Linker | AGTCGVLLLSLVITLYCKSRQTPPPGGPGDEMEEC | |
| P2A | SQHLPGGSERTMPRIPTLKNLEDLVTEYHGNFSAW | |
| Linker | SGVSKGLAESLQPDYSERLCLVSEIPPKGGALGEG | |
| GM-CSF leader sequence | PGASPCNQHSPYWAPPCYTLKPET | |
| HA Tag | | |
| Disulfide linker | | |
| EE leucine zipper | | |
| CD8α hinge | | |
| CD8α transmembrane | | |
| domain | | |
| IL15Rα transition peptide | | |
| Linker | | |
| HCV 4a4b cleavage site | | |
| Linker | | |
| γ-chain ICD | | |
| #529 Bicistronic IL2 CCR with | MLLLVTSLLLCELPHPAFLLIPDYKDDDDKCGGKGG | 10 |
| 4a4b cleavage site (FLAG Tag.RR | GLEIRAAFLRRRNTALRTRVAELRQRVQRARNRVS | |
| zipper. CD8α H/TM. IL2Rβ | QYRTRYGPLTTTPAPRPPTPAPTIASQPLSLRPEAC | |
| ICD.P2A.HA Tag. EE zipper.CD8α | RPAAGGAVHTRGLDFACDIYIWAPLAGTCGVLLLSL | |
| H/TM.IL15Rα transition peptide. | VITLYCNCRNTGPWLKKVLKCNTPDPSKFFSQLSS | |
| HCV 4a4b cleavage site. γ-chain | EHGGDVQKWLSSPFPSSSFSPGGLAPEISPLEVLE | |
| ICD) | RDKVTQLLLQQDKVPEPASLSSNHSLTSCFTNQGY | |
| GM-CSF leader sequence | FFFHLPDALEIEACQVYFTYDPYSEEDPDEGVAGA | |
| FLAG Tag | PTGSSPQPLQPLSGEDDAYCTFPSRDDLLLFSPSL | |
| Disulfide linker | LGGPSPPSTAPGGSGAGEERMPPSLQERVPRDW | |
| RR leucine zipper | DPQPLGPPTPGVPDLVDFQPPPELVLREAGEEVPD | |
| CD8α hinge | AGPREGVSFPWSRPPGQGEFRALNARLPLNTDAY | |
| CD8α transmembrane | LSLQELQGQDPTHLVSIGATNFSLLKQAGDVEENP | |
| domain | GPGSTMLLLVTSLLLCELPHPAFLLIPYPYDVPDYA | |
| IL2Rβ intracellular domain | CGGSSGLEIEAAFLEQENTALETEVAELEQEVQRL | |
| Linker | ENEVSQYETRYGPLGGGKTTTPAPRPPTPAPTIAS | |
| P2A | QPLSLRPEACRPAAGGAVHTRGLDFACDIYIWAPL | |
| Linker | AGTCGVLLLSLVITLYCKSRQTPPPGGPGECTTPC | |
| GM-CSF leader sequence | SGSWLPGGSERTMPRIPTLKNLEDLVTEYHGNFSA | |
| HA Tag | WSGVSKGLAESLQPDYSERLCLVSEIPPKGGALGE | |
| Disulfide linker | GPGASPCNQHSPYWAPPCYTLKPET | |
| EE leucine zipper | | |
| CD8α hinge | | |
| CD8α transmembrane | | |
| domain | | |
| IL15Rα transition peptide | | |
| Linker | | |
| HCV 4b5a cleavage site | | |
| Linker | | |
| γ-chain ICD | | |
| # 523 HCV NS3 protease | MGTSLLCWMALCLLGADHADACPYSNPSLCSGGG | 11 |
| TCRB Leader | GSELPTQGTFSNVSTNVSPAKPTTTACPYSNPSLC | |
| RQR8 Tag | SGGGGSPAPRPPTPAPTIASQPLSLRPEACRPAAG | |
| CD8α Hinge | GAVHTRGLDFACD | |
| CD8α Tm | IYIWAPLAGTCGVLLLSLVITLYCNHRNRRRVCKCP | |
| CD8α intracellular linker | RPVVGSSGNSSGGSTGCVVIVGRIVLSGSGTSAPI | |
| NS4A cofactor domain | TAYAQQTRGLLGCIITSLTGRDKNQVEGEVQIMSTA | |
| linker | TQTFLATCINGVCWTVYHGAGTRTIASPKGPVIQM | |
| HCV NS3 protease | YTNVDQDLVGWPAPQGSRSLTPCTCGSSDLYLVT | |
| NS3 helicase fragment | RHADVIPVRRRGDGRGSLLSPRPISYLKGSSGGPL | |
| Linker | LCPAGHAVGLFRAAVCTRGVAKAVDFIPVENLETT | |
| | MRSPVFTDNSSPPAVTLTHNAS | |
| #559 Bicistronic IL2Rβ with 4b5a | MLLLVTSLLLCELPHPAFLLIPDYKDDDDKCGGKGG | 12 |
| cleavage site (FLAG Tag.RR | GLEIRAAFLRRRNTALRTRVAELRQRVQRARNRVS | |
| zipper. CD8α H/TM.HCV 4b5a | QYRTRYGPLTTTPAPRPPTPAPTIASQPLSLRPEAC | |
| cleavage site. IL2Rβ ICD.P2A.HCV | RPAAGGAVHTRGLDFACDIYIWAPLAGTCGVLLLSL | |
| protease) | VITLYCKPGGPGECTTPCSGSWLAAANCRNTGPW | |
| GM-CSF leader sequence | LKKVLKCNTPDPSKFFSQLSSEHGGDVQKWLSSP | |
| FLAG Tag | FPSSSFSPGGLAPEISPLEVLERDKVTQLLLQQDKV | |
| Disulfide linker | PEPASLSSNHSLTSCFTNQGYFFFHLPDALEIEACQ | |
| RR leucine zipper | VYFTYDPYSEEDPDEGVAGAPTGSSPQPLQPLSG | |
| CD8α hinge | EDDAYCTFPSRDDLLLFSPSLLGGPSPPSTAPGGS | |
| CD8α transmembrane | GAGEERMPPSLQERVPRDWDPQPLGPPTPGVPD | |
| domain | LVDFQPPPELVLREAGEEVPDAGPREGVSFPWSR | |
| Linker | PPGQGEFRALNARLPLNTDAYLSLQELQGQDPTHL | |
| HCV 4b5a cleavage site | VSIGATNFSLLKQAGDVEENPGPGSTMGTSLLCW | |
| Linker | MALCLLGADHADACPYSNPSLCSGGGGSELPTQG | |
| IL2Rβ intracellular domain | TFSNVSTNVSPAKPTTTACPYSNPSLCSGGGGSPA | |
| Linker | PRPPTPAPTIASQPLSLRPEACRPAAGGAVHTRGL | |
| P2A | DFACDIYIWAPLAGTCGVLLLSLVITLYCNHRNRRR | |
| Linker | VCKCPRPVVGSSGNSSGGSTGCVVIVGRIVLSGSG | |
| TCRB Leader | TSAPITAYAQQTRGLLGCIITSLTGRDKNQVEGEVQ | |
| RQR8 Tag | IMSTATQTFLATCINGVCWTVYHGAGTRTIASPKGP | |
| CD8α Hinge | VIQMYTNVDQDLVGWPAPQGSRSLTPCTCGSSDL | |
| CD8α Tm | YLVTRHADVIPVRRRGDGRGSLLSPRPISYLKGSS | |
| CD8α intracellular linker | GGPLLCPAGHAVGLFRAAVCTRGVAKAVDFIPVEN | |
| NS4A cofactor domain | LETTMRSPVFTDNSSPPAVTLTHNAS | |
| linker | | |
| HCV NS3 protease | | |
| NS3 helicase fragment | | |
| Linker | | |
| #560 Bicistronic gamma chain with | MLLLVTSLLLCELPHPAFLLIPYPYDVPDYACGGSS | 13 |
| 4b5a cleavage site (HA Tag.EE | GLEIEAAFLEQENTALETEVAELEQEVQRLENEVSQ | |
| zipper. CD8α H/TM.HCV 4b5a | YETRYGPLGGGKTTTPAPRPPTPAPTIASQPLSLR | |
| cleavage site. γ-chain | PEACRPAAGGAVHTRGLDFACDIYIWAPLAGTCGV | |
| ICD.P2A.HCV protease) | LLLSLVITLYCKSRQTPPPGGPGECTTPCSGSWLP | |
| GM-CSF leader sequence | GGSERTMPRIPTLKNLEDLVTEYHGNFSAWSGVSK | |
| HA Tag | GLAESLQPDYSERLCLVSEIPPKGGALGEGPGASP | |
| Disulfide linker | CNQHSPYWAPPCYTLKPETSIGATNFSLLKQAGDV | |
| EE leucine zipper | EENPGPGSTMGTSLLCWMALCLLGADHADACPYS | |
| CD8α hinge | NPSLCSGGGGSELPTQGTFSNVSTNVSPAKPTTTA | |
| CD8α transmembrane | CPYSNPSLCSGGGGSPAPRPPTPAPTIASQPLSLR | |
| domain | PEACRPAAGGAVHTRGLDFACDIYIWAPLAGTCGV | |
| IL15Rα transition peptide | LLLSLVITLYCNHRNRRRVCKCPRPVVGSSGNSSG | |
| Linker | GSTGCVVIVGRIVLSGSGTSAPITAYAQQTRGLLGC | |
| HCV 4b5a cleavage site | IITSLTGRDKNQVEGEVQIMSTATQTFLATCINGVC | |
| Linker | WTVYHGAGTRTIASPKGPVIQMYTNVDQDLVGWP | |
| γ-chain ICD | APQGSRSLTPCTCGSSDLYLVTRHADVIPVRRRGD | |
| Linker | GRGSLLSPRPISYLKGSSGGPLLCPAGHAVGLFRA | |
| P2A | AVCTRGVAKAVDFIPVENLETTMRSPVFTDNSSPP | |
| Linker | AVTLTHNAS | |
| TCRß Leader | | |
| RQR8 Tag | | |
| CD8α Hinge | | |
| CD8α | | |
| Tm | | |
| CD8α intracellular linker | | |
| NS4A cofactor domain | | |
| Linker | | |
| HCV NS3 protease | | |
| NS3 helicase fragment | | |
| Linker | | |
| #864 Bicistronic gamma chain with | MLLLVTSLLLCELPHPAFLLIPYPYDVPDYACGGSS | 14 |
| 4b5a cleavage site (HA Tag.EE | GLEIEAAFLEQENTALETEVAELEQEVQRLENEVSQ | |
| zipper. CD8α H/TM.HCV 4b5a | YETRYGPLGGGKTTTPAPRPPTPAPTIASQPLSLR | |
| cleavage site. γ-chain | PEACRPAAGGAVHTRGLDFACDIYIWAPLAGTCGV | |
| ICD.P2A.HCV protease.P2A. Flag | LLLSLVITLYCKSRQTPPPGGPGECTTPCSGSWLP | |
| Tag. RR zipper. CD8α H/TM.IL2Rβ | GGSERTMPRIPTLKNLEDLVTEYHGNFSAWSGVSK | |
| ICD) | GLAESLQPDYSERLCLVSEIPPKGGALGEGPGASP | |
| GM-CSF leader sequence | CNQHSPYWAPPCYTLKPETSIGATNFSLLKQAGDV | |
| HA Tag | EENPGPGSTMGTSLLCWMALCLLGADHADACPYS | |
| Disulfide linker | NPSLCSGGGGSELPTQGTFSNVSTNVSPAKPTTTA | |
| EE leucine zipper | CPYSNPSLCSGGGGSPAPRPPTPAPTIASQPLSLR | |
| CD8α | PEACRPAAGGAVHTRGLDFACDIYIWAPLAGTCGV | |
| hinge | LLLSLVITLYCNHRNRRRVCKCPRPVVGSSGNSSG | |
| CD8α | GSTGCVVIVGRIVLSGSGTSAPITAYAQQTRGLLGC | |
| transmembrane | IITSLTGRDKNQVEGEVQIMSTATQTFLATCINGVC | |
| domain | WTVYHGAGTRTIASPKGPVIQMYTNVDQDLVGWP | |
| IL15Rα transition peptide | APQGSRSLTPCTCGSSDLYLVTRHADVIPVRRRGD | |
| Linker | GRGSLLSPRPISYLKGSSGGPLLCPAGHAVGLFRA | |
| HCV 4b5a cleavage site | AVCTRGVAKAVDFIPVENLETTMRSPVFTDNSSPP | |
| Linker | AVTLTHNASATNFSLLKQAGDVEENPGPGSTMLLL | |
| γ-chain ICD | VTSLLLCELPHPAFLLIPDYKDDDDKCGGKGGGLEI | |
| Linker | RAAFLRRRNTALRTRVAELRQRVQRARNRVSQYR | |
| P2A | TRYGPLTTTPAPRPPTPAPTIASQPLSLRPEACRPA | |
| Linker | AGGAVHTRGLDFACDIYIWAPLAGTCGVLLLSLVIT | |
| TCRB Leader | LYCNCRNTGPWLKKVLKCNTPDPSKFFSQLSSEH | |
| RQR8 Tag | GGDVQKWLSSPFPSSSFSPGGLAPEISPLEVLERD | |
| CD8α Hinge | KVTQLLLQQDKVPEPASLSSNHSLTSCFTNQGYFF | |
| CD8α Tm | FHLPDALEIEACQVYFTYDPYSEEDPDEGVAGAPT | |
| CD8α intracellular linker | GSSPQPLQPLSGEDDAYCTFPSRDDLLLFSPSLLG | |
| NS4A cofactor domain | GPSPPSTAPGGSGAGEERMPPSLQERVPRDWDP | |
| Linker | QPLGPPTPGVPDLVDFQPPPELVLREAGEEVPDA | |
| HCV NS3 protease | GPREGVSFPWSRPPGQGEFRALNARLPLNTDAYL | |
| NS3 helicase fragment | SLQELQGQDPTHLV | |
| Linker | | |
| P2A | | |
| Linker | | |
| GM-CSF leader sequence | | |
| FLAG Tag | | |
| Disulfide linker | | |
| RR leucine zipper | | |
| CD8α hinge | | |
| CD8α transmembrane | | |
| domain | | |
| IL2Rβ intracellular domain | | |
| 907 Gamma chain without leucine | MLLLVTSLLLCELPHPAFLLIPYPYDVPDYACGGTT | 15 |
| zipper (HA Tag.CD8α H/TM.IL15Rα | TPAPRPPTPAPTIASQPLSLRPEACRPAAGGAVHT | |
| transition peptide.γ-chain ICD) | RGLDFACDIYIWAPLAGTCGVLLLSLVITLYCKSRQT | |
| GM-CSF leader sequence | PPERTMPRIPTLKNLEDLVTEYHGNFSAWSGVSKG | |
| HA Tag | LAESLQPDYSERLCLVSEIPPKGGALGEGPGASPC | |
| Disulfide linker | NQHSPYWAPPCYTLKPET | |
| CD8α hinge | | |
| CD8α transmembrane | | |
| domain | | |
| IL15Rα transition peptide | | |
| γ-chain ICD | | |
| 908 IL2Rβ chain without leucine | MLLLVTSLLLCELPHPAFLLIPYPYDVPDYACGGTT | 16 |
| zipper (HA Tag.CD8α H/TM.IL2Rβ | TPAPRPPTPAPTIASQPLSLRPEACRPAAGGAVHT | |
| ICD) | RGLDFACDIYIWAPLAGTCGVLLLSLVITLYCNCRN | |
| GM-CSF leader sequence | TGPWLKKVLKCNTPDPSKFFSQLSSEHGGDVQKW | |
| HA Tag | LSSPFPSSSFSPGGLAPEISPLEVLERDKVTQLLLQ | |
| Disulfide linker | QDKVPEPASLSSNHSLTSCFTNQGYFFFHLPDALEI | |
| CD8α hinge | EACQVYFTYDPYSEEDPDEGVAGAPTGSSPQPLQ | |
| CD8α transmembrane | PLSGEDDAYCTFPSRDDLLLFSPSLLGGPSPPSTA | |
| domain | PGGSGAGEERMPPSLQERVPRDWDPQPLGPPTP | |
| IL2Rβ ICD | GVPDLVDFQPPPELVLREAGEEVPDAGPREGVSF | |
| | PWSRPPGQGEFRALNARLPLNTDAYLSLQELQGQ | |
| | DPTHLV | |
| # OF CD22 CAR (CD22.BBz) | MLLLVTSLLLCELPHPAFLLIPQVQLQQSGPGLVKP | 17 |
| GMCSF leader sequence | SQTLSLTCAISGDSVSSNSAAWNWIRQSPSRGLE | |
| CD22 scFv | WLGRTYYRSKWYNDYAVSVKSRITINPDTSKNQFS | |
| Linker | LQLNSVTPEDTAVYYCAREVTGDLEDAFDIWGQGT | |
| CD8α hinge | MVTVSSGGGGSDIQMTQSPSSLSASVGDRVTITCR | |
| CD8α transmembrane | ASQTIWSYLNWYQQRPGKAPNLLIYAASSLQSGVP | |
| 4-1 BB ICD | SRFSGRGSGTDFTLTISSLQAEDFATYYCQQSYSIP | |
| CD3z ICD | QTFGQGTKLEIKAAATTTPAPRPPTPAPTIASQPLS | |
| | LRPEACRPAAGGAVHTRGLDFACDIYIWAPLAGTC | |
| | GVLLLSLVITLYCKRGRKKLLYIFKQPFMRPVQTTQ | |
| | EEDGCSCRFPEEEEGGCELRVKFSRSADAPAYKQ | |
| | GQNQLYNELNLGRREEYDVLDKRRGRDPEMGGK | |
| | PRRKNPQEGLYNELQKDKMAEAYSEIGMKGERRR | |
| | GKGHDGLYQGLSTATKDTYDALHMQALPPR | |
| # 931 Bicistronic CD22 CAR with | MLLLVTSLLLCELPHPAFLLIPQVQLQQSGPGLVKP | 18 |
| IL2Rβ chain (CD22.BBz. P2A. | SQTLSLTCAISGDSVSSNSAAWNWIRQSPSRGLE | |
| FLAG-RR zipper CD8α H/TM | WLGRTYYRSKWYNDYAVSVKSRITINPDTSKNQFS | |
| IL2Rβ) | LQLNSVTPEDTAVYYCAREVTGDLEDAFDIWGQGT | |
| GMCSF leader sequence | MVTVSSGGGGSDIQMTQSPSSLSASVGDRVTITCR | |
| CD22 scFv | ASQTIWSYLNWYQQRPGKAPNLLIYAASSLQSGVP | |
| Linker | SRFSGRGSGTDFTLTISSLQAEDFATYYCQQSYSIP | |
| CD8α hinge | QTFGQGTKLEIKAAATTTPAPRPPTPAPTIASQPLS | |
| CD8α transmembrane | LRPEACRPAAGGAVHTRGLDFACDIYIWAPLAGTC | |
| 4-1BB ICD | GVLLLSLVITLYCKRGRKKLLYIFKQPFMRPVQTTQ | |
| CD3z ICD | EEDGCSCRFPEEEEGGCELRVKFSRSADAPAYKQ | |
| Linker | GQNQLYNELNLGRREEYDVLDKRRGRDPEMGGK | |
| P2A | PRRKNPQEGLYNELQKDKMAEAYSEIGMKGERRR | |
| Linker | GKGHDGLYQGLSTATKDTYDALHMQALPPRSIGAT | |
| GM-CSF leader sequence | NFSLLKQAGDVEENPGPGSTMLLLVTSLLLCELPH | |
| FLAG Tag | PAFLLIPDYKDDDDKCGGKGGGLEIRAAFLRRRNT | |
| Disulfide linker | ALRTRVAELRQRVQRARNRVSQYRTRYGPLTTTP | |
| RR leucine zipper | APRPPTPAPTIASQPLSLRPEACRPAAGGAVHTRG | |
| CD8α hinge | LDFACDIYIWAPLAGTCGVLLLSLVITLYCNCRNTGP | |
| CD8α transmembrane | WLKKVLKCNTPDPSKFFSQLSSEHGGDVQKWLSS | |
| domain | PFPSSSFSPGGLAPEISPLEVLERDKVTQLLLQQDK | |
| IL2Rβ intracellular domain | VPEPASLSSNHSLTSCFTNQGYFFFHLPDALEIEAC | |
| | QVYFTYDPYSEEDPDEGVAGAPTGSSPQPLQPLS | |
| | GEDDAYCTFPSRDDLLLFSPSLLGGPSPPSTAPGG | |
| | SGAGEERMPPSLQERVPRDWDPQPLGPPTPGVP | |
| | DLVDFQPPPELVLREAGEEVPDAGPREGVSFPWS | |
| | RPPGQGEFRALNARLPLNTDAYLSLQELQGQDPT | |
| | HLV | |
| #939 Gamma chain with 4b5a | MLLLVTSLLLCELPHPAFLLIPYPYDVPDYACGGSS | 19 |
| cleavage site and HCV protease | GLEIEAAFLEQENTALETEVAELEQEVQRLENEVSQ | |
| fusion (HA Tag. EE zipper. CD8α | YETRYGPLGGGKTTTPAPRPPTPAPTIASQPLSLR | |
| H/TM.HCV 4b5a cleavage site. γ- | PEACRPAAGGAVHTRGLDFACDIYIWAPLAGTCGV | |
| chain ICD.HCV protease) | LLLSLVITLYCKSRQTPPGGPGECTTPCSGSWLPG | |
| GM-CSF leader sequence | GSERTMPRIPTLKNLEDLVTEYHGNFSAWSGVSKG | |
| HA Tag | LAESLQPDYSERLCLVSEIPPKGGALGEGPGASPC | |
| Disulfide linker | NQHSPYWAPPCYTLKPETGSTGGGSGGGSGGGS | |
| EE leucine zipper | GSSGNSSGGSTGCVVIVGRIVLSGSGTSAPITAYA | |
| CD8α | QQTRGLLGCIITSLTGRDKNQVEGEVQIMSTATQTF | |
| hinge | LATCINGVCWTVYHGAGTRTIASPKGPVIQMYTNV | |
| CD8α transmembrane | DQDLVGWPAPQGSRSLTPCTCGSSDLYLVTRHAD | |
| domain | VIPVRRRGDGRGSLLSPRPISYLKGSSGGPLLCPA | |
| IL15Rα transition peptide | GHAVGLFRAAVCTRGVAKAVDFIPVENLETTMRSP | |
| Linker | VFTDNSSPPAVTLTHNAS | |
| HCV 4b5a cleavage site | | |
| Linker | | |
| γ-chain ICD | | |
| Linker | | |
| NS4A cofactor domain | | |
| Linker | | |
| HCV NS3 protease | | |
| NS3 helicase fragment | | |
| Linker | | |
| # 941 Bicistronic constitutive IL9 | MLLLVTSLLLCELPHPAFLLIPDYKDDDDKCGGKGG | 20 |
| CCR (FLAG Tag.RR zipper. CD8α | GLEIRAAFLRRRNTALRTRVAELRQRVQRARNRVS | |
| H/TM. IL9R ICD.P2A.HA Tag.EE | QYRTRYGPLTTTPAPRPPTPAPTIASQPLSLRPEAC | |
| zipper.CD8α H/TM.IL15Rα | RPAAGGAVHTRGLDFACDIYIWAPLAGTCGVLLLSL | |
| transition peptide. γ-chain ICD) | VITLYCKLSPRVKRIFYQNVPSPAMFFQPLYSVHNG | |
| GM-CSF leader sequence | NFQTWMGAHGAGVLLSQDCAGTPQGALEPCVQE | |
| FLAG Tag | ATALLTCGPARPWKSVALEEEQEGPGTRLPGNLS | |
| Disulfide linker | SEDVLPAGCTEWRVQTLAYLPQEDWAPTSLTRPA | |
| RR leucine zipper | PPDSEGSRSSSSSSSSNNNNYCALGCYGGWHLS | |
| CD8α hinge | ALPGNTQSSGPIPALACGLSCDHQGLETQQGVAW | |
| CD8α transmembrane | VLAGHCQRPGLHEDLQGMLLPSVLSKARSWTFSIG | |
| domain | ATNFSLLKQAGDVEENPGPGSTMLLLVTSLLLCELP | |
| IL9R intracellular domain | HPAFLLIPYPYDVPDYACGGSSGLEIEAAFLEQENT | |
| Linker | ALETEVAELEQEVQRLENEVSQYETRYGPLGGGK | |
| P2A | TTTPAPRPPTPAPTIASQPLSLRPEACRPAAGGAV | |
| Linker | HTRGLDFACDIYIWAPLAGTCGVLLLSLVITLYCKSR | |
| GM-CSF leader sequence | QTPPERTMPRIPTLKNLEDLVTEYHGNFSAWSGVS | |
| HA Tag | KGLAESLQPDYSERLCLVSEIPPKGGALGEGPGAS | |
| Disulfide linker | PCNQHSPYWAPPCYTLKPET | |
| EE leucine zipper | | |
| CD8α hinge | | |
| CD8α transmembrane | | |
| domain | | |
| IL15Rα transition peptide | | |
| γ-chain ICD | | |
| # 942 Bicistronic constitutive IL7 | MLLLVTSLLLCELPHPAFLLIPDYKDDDDKCGGKGG | 21 |
| CCR (FLAG Tag.RR zipper. CD8α | GLEIRAAFLRRRNTALRTRVAELRQRVQRARNRVS | |
| H/TM. IL7R ICD.P2A.HA Tag.EE | QYRTRYGPLTTTPAPRPPTPAPTIASQPLSLRPEAC | |
| zipper.CD8α H/TM.IL15Rα | RPAAGGAVHTRGLDFACDIYIWAPLAGTCGVLLLSL | |
| transition peptide.γ-chain ICD) | VITLYCKKRIKPIVWPSLPDHKKTLEHLCKKPRKNL | |
| GM-CSF leader sequence | NVSFNPESFLDCQIHRVDDIQARDEVEGFLQDTFPQ | |
| FLAG Tag | QLEESEKQRLGGDVQSPNCPSEDVVITPESFGRD | |
| Disulfide linker | SSLTCLAGNVSACDAPILSSSRSLDCRESGKNGPH | |
| RR leucine zipper | VYQDLLLSLGTTNSTLPPPFSLQSGILTLNPVAQGQ | |
| CD8α | PILTSLGSNQEEAYVTMSSFYQNQSIGATNFSLLKQ | |
| hinge | AGDVEENPGPGSTMLLLVTSLLLCELPHPAFLLIPY | |
| CD8α transmembrane | PYDVPDYACGGSSGLEIEAAFLEQENTALETEVAE | |
| domain | LEQEVQRLENEVSQYETRYGPLGGGKTTTPAPRP | |
| IL7R intracellular domain | PTPAPTIASQPLSLRPEACRPAAGGAVHTRGLDFA | |
| Linker | CDIYIWAPLAGTCGVLLLSLVITLYCKSRQTPPERT | |
| P2A | MPRIPTLKNLEDLVTEYHGNFSAWSGVSKGLAESL | |
| Linker | QPDYSERLCLVSEIPPKGGALGEGPGASPCNQHS | |
| GM-CSF leader sequence | PYWAPPCYTLKPET | |
| HA Tag | | |
| Disulfide linker | | |
| EE leucine zipper | | |
| CD8α hinge | | |
| CD8α transmembrane | | |
| domain | | |
| IL15Rα transition peptide | | |
| γ-chain ICD | | |
| # 943 Bicistronic CD22 CAR with | MLLLVTSLLLCELPHPAFLLIPQVQLQQSGPGLVK | 22 |
| IL9R chain (CD22.BBz.P2A. FLAG- | PSQTLSLTCAISGDSVSSNSAAWNWIRQSPSRGLE | |
| RR zipper CD8α H/TM IL9R ICD) | WLGRTYYRSKWYNDYAVSVKSRITINPDTSKNQFS | |
| GMCSF leader sequence | LQLNSVTPEDTAVYYCAREVTGDLEDAFDIWGQGT | |
| CD22 scFv | MVTVSSGGGGSDIQMTQSPSSLSASVGDRVTITCR | |
| Linker | ASQTIWSYLNWYQQRPGKAPNLLIYAASSLQSGVP | |
| CD8α hinge | SRFSGRGSGTDFTLTISSLQAEDFATYYCQQSYSIP | |
| CD8α transmembrane | QTFGQGTKLEIKAAATTTPAPRPPTPAPTIASQPLS | |
| 4-1BB ICD | LRPEACRPAAGGAVHTRGLDFACDIYIWAPLAGTC | |
| CD3z ICD | GVLLLSLVITLYCKRGRKKLLYIFKQPFMRPVQTT | |
| Linker | QEEDGCSCRFPEEEEGGCELRVKFSRSADAPAYKQ | |
| P2A | GQNQLYNELNLGRREEYDVLDKRRGRDPEMGGK | |
| Linker | PRRKNPQEGLYNELQKDKMAEAYSEIGMKGERRR | |
| GM-CSF leader sequence | GKGHDGLYQGLSTATKDTYDALHMQALPPRSIGAT | |
| FLAG Tag | NFSLLKQAGDVEENPGPGSTMLLLVTSLLLCELPH | |
| Disulfide linker | PAFLLIPDYKDDDDKCGGKGGGLEIRAAFLRRRNT | |
| RR leucine zipper | ALRTRVAELRQRVQRARNRVSQYRTRYGPLTTTP | |
| CD8α hinge | APRPPTPAPTIASQPLSLRPEACRPAAGGAVHTRG | |
| CD8α transmembrane | LDFACDIYIWAPLAGTCGVLLLSLVITLYCKLSPR | |
| domain | VKRIFYQNVPSPAMFFQPLYSVHNGNFQTWMGAHG | |
| IL9R intracellular domain | AGVLLSQDCAGTPQGALEPCVQEATALLTCGPARP | |
| | WKSVALEEEQEGPGTRLPGNLSSEDVLPAGCTEW | |
| | RVQTLAYLPQEDWAPTSLTRPAPPDSEGSRSSSS | |
| | SSSSNNNNYCALGCYGGWHLSALPGNTQSSGPIP | |
| | ALACGLSCDHQGLETQQGVAWVLAGHCQRPGLH | |
| | EDLQGMLLPSVLSKARSWTE | |
| # 944 Bicistronic CD22 CAR with | MLLLVTSLLLCELPHPAFLLIPQVQLQQSGPGLVKP | 23 |
| IL7R chain (CD22.BBz.P2A. FLAG- | SQTLSLTCAISGDSVSSNSAAWNWIRQSPSRGLE | |
| RR zipper CD8α H/TM IL7R ICD) | WLGRTYYRSKWYNDYAVSVKSRITINPDTSKNQFS | |
| GMCSF leader sequence | LQLNSVTPEDTAVYYCAREVTGDLEDAFDIWGQGT | |
| CD22 scFv | MVTVSSGGGGSDIQMTQSPSSLSASVGDRVTITCR | |
| Linker | ASQTIWSYLNWYQQRPGKAPNLLIYAASSLQSGVP | |
| CD8α hinge | SRFSGRGSGTDFTLTISSLQAEDFATYYCQQSYSIP | |
| CD8α transmembrane | QTFGQGTKLEIKAAATTTPAPRPPTPAPTIASQPLS | |
| 4-1 BB ICD | LRPEACRPAAGGAVHTRGLDFACDIYIWAPLAGTC | |
| CD3z ICD | GVLLLSLVITLYCKRGRKKLLYIFKQPFMRPVQTTQ | |
| Linker | EEDGCSCRFPEEEEGGCELRVKFSRSADAPAYKQ | |
| P2A | GQNQLYNELNLGRREEYDVLDKRRGRDPEMGGK | |
| Linker | PRRKNPQEGLYNELQKDKMAEAYSEIGMKGERRR | |
| GM-CSF leader sequence | GKGHDGLYQGLSTATKDTYDALHMQALPPRSIGAT | |
| FLAG Tag | NFSLLKQAGDVEENPGPGSTMLLLVTSLLLCELPH | |
| Disulfide linker | PAFLLIPDYKDDDDKCGGKGGGLEIRAAFLRRRNT | |
| RR leucine zipper | ALRTRVAELRQRVQRARNRVSQYRTRYGPLTTTP | |
| CD8α hinge | APRPPTPAPTIASQPLSLRPEACRPAAGGAVHTRGL | |
| CD8α transmembrane | DFACDIYIWAPLAGTCGVLLLSLVITLYCKKRIKPI | |
| domain | VWPSLPDHKKTLEHLCKKPRKNLNVSFNPESFLDCQ | |
| IL7R intracellular domain | IHRVDDIQARDEVEGFLQDTFPQQLEESEKQRLGG | |
| | DVQSPNCPSEDVVITPESFGRDSSLTCLAGNVSAC | |
| | DAPILSSSRSLDCRESGKNGPHVYQDLLLSLGTTN | |
| | STLPPPFSLQSGILTLNPVAQGQPILTSLGSNQEEA | |
| | YVTMSSFYQNQ | |
| # 945 Bicistronic CD22 CAR with | MLLLVTSLLLCELPHPAFLLIPQVQLQQSGPGLVK | 24 |
| no ICD on the CCR | PSQTLSLTCAISGDSVSSNSAAWNWIRQSPSRGLE | |
| (CD22.BBz.P2A. FLAG-RR zipper | WLGRTYYRSKWYNDYAVSVKSRITINPDTSKNQFS | |
| CD8α H/TM) | LQLNSVTPEDTAVYYCAREVTGDLEDAFDIWGQGT | |
| GMCSF leader sequence | MVTVSSGGGGSDIQMTQSPSSLSASVGDRVTITCR | |
| CD22 scFv | ASQTIWSYLNWYQQRPGKAPNLLIYAASSLQSGVP | |
| Linker | SRFSGRGSGTDFTLTISSLQAEDFATYYCQQSYSIP | |
| CD8α hinge | QTFGQGTKLEIKAAATTTPAPRPPTPAPTIASQPLS | |
| CD8α transmembrane | LRPEACRPAAGGAVHTRGLDFACDIYIWAPLAGTC | |
| 4-1BB ICD | GVLLLSLVITLYCKRGRKKLLYIFKQPFMRPVQTTQ | |
| CD3z ICD | EEDGCSCRFPEEEEGGCELRVKFSRSADAPAYKQ | |
| Linker | GQNQLYNELNLGRREEYDVLDKRRGRDPEMGGK | |
| P2A | PRRKNPQEGLYNELQKDKMAEAYSEIGMKGERRR | |
| Linker | GKGHDGLYQGLSTATKDTYDALHMQALPPRSIGAT | |
| GM-CSF leader sequence | NFSLLKQAGDVEENPGPGSTMLLLVTSLLLCELPH | |
| FLAG Tag | PAFLLIPDYKDDDDKCGGKGGGLEIRAAFLRRRNT | |
| Disulfide linker | ALRTRVAELRQRVQRARNRVSQYRTRYGPLTTTP | |
| Disulfide linker | APRPPTPAPTIASQPLSLRPEACRPAAGGAVHTRG | |
| RR leucine zipper | LDFACDIYIWAPLAGTCGVLLLSLVITLYC | |
| CD8α hinge | | |
| CD8α transmembrane | | |
| domain | | |
| # 1077 Bicistronic CD22 CAR with | MLLLVTSLLLCELPHPAFLLIPQVQLQQSGPGLVK | 25 |
| ILI8RA chain (CD22.BBz.P2A. | PSQTLSLTCAISGDSVSSNSAAWNWIRQSPSRGLE | |
| FLAG-RR zipper CD8α H/TM | WLGRTYYRSKWYNDYAVSVKSRITINPDTSKNQFS | |
| IL18RA ICD) | LQLNSVTPEDTAVYYCAREVTGDLEDAFDIWGQGT | |
| GMCSF leader sequence | MVTVSSGGGGSDIQMTQSPSSLSASVGDRVTITCR | |
| CD22 scFv | ASQTIWSYLNWYQQRPGKAPNLLIYAASSLQSGVP | |
| Linker | SRFSGRGSGTDFTLTISSLQAEDFATYYCQQSYSIP | |
| CD8α hinge | QTFGQGTKLEIKAAATTTPAPRPPTPAPTIASQPLS | |
| CD8α transmembrane | LRPEACRPAAGGAVHTRGLDFACDIYIWAPLAGTC | |
| 4-1BB ICD | GVLLLSLVITLYCKRGRKKLLYIFKQPFMRPVQTTQ | |
| CD3z ICD | EEDGCSCRFPEEEEGGCELRVKFSRSADAPAYKQ | |
| Linker | GQNQLYNELNLGRREEYDVLDKRRGRDPEMGGK | |
| P2A | PRRKNPQEGLYNELQKDKMAEAYSEIGMKGERRR | |
| Linker | GKGHDGLYQGLSTATKDTYDALHMQALPPRSIGAT | |
| GM-CSF leader sequence | NFSLLKQAGDVEENPGPGSTMLLLVTSLLLCELPH | |
| FLAG Tag | PAFLLIPDYKDDDDKCGGKGGGLEIRAAFLRRRNT | |
| Disulfide linker | ALRTRVAELRQRVQRARNRVSQYRTRYGPLTTTP | |
| RR leucine zipper | APRPPTPAPTIASQPLSLRPEACRPAAGGAVHTRG | |
| CD8α hinge | LDFACDIYIWAPLAGTCGVLLLSLVITLYCYRVDLVL | |
| CD8α transmembrane | FYRHLTRRDETLTDGKTYDAFVSYLKECRPENGEE | |
| domain | HTFAVEILPRVLEKHFGYKLCIFERDVVPGGAVVDE | |
| IL18RA intracellular domain | IHSLIEKSRRLIIVLSKSYMSNEVRYELESGLHEAL | |
| | VERKIKIILIEFTPVTDFTFLPQSLKLLKSHRVLKW | |
| | KADKSLSYNSRFWKNLLYLMPAKTVKPGRDEPEVLP | |
| | VLSES | |
| 1078 HA Tag.EE zipper.CD8α | MLLLVTSLLLCELPHPAFLLIPYPYDVPDYACGGSS | 26 |
| H/TM.IL 15Ra transition | GLEIEAAFLEQENTALETEVAELEQEVQRLENEVSQ | |
| peptide.IL18RB ICD | YETRYGPLGGGKTTTPAPRPPTPAPTIASQPLSLR | |
| GM-CSF leader sequence | PEACRPAAGGAVHTRGLDFACDIYIWAPLAGTCGV | |
| HA Tag | LLLSLVITLYCKSRQTPPSALLYRHWIEIVLLYRTYQ | |
| Disulfide linker | SKDQTLGDKKDFDAFVSYAKWSSFPSEATSSLSEE | |
| EE leucine zipper | HLALSLFPDVLENKYGYSLCLLERDVAPGGVYAEDI | |
| CD8α hinge | VSIIKRSRRGIFILSPNYVNGPSIFELQAAVNLALDD | |
| CD8α transmembrane | QTLKLILIKFCYFQEPESLPHLVKKALRVLPTVTWR | |
| domain | GLKSVPPNSRFWAKMRYHMPVKNSQGFTWNQLRI | |
| IL15Rα transition peptide | TSRIFQWKGLSRTETTGRSSQPKEW | |
| IL18RB ICD | | |
| #1079 Bicistronic IL18RB with 4b5a | MLLLVTSLLLCELPHPAFLLIPYPYDVPDYACGGSS | 27 |
| cleavage site (HA Tag.EE zipper. | GLEIEAAFLEQENTALETEVAELEQEVQRLENEVSQ | |
| CD8α H/TM.HCV 4b5a cleavage | YETRYGPLGGGKTTTPAPRPPTPAPTIASQPLSLR | |
| site.IL18RB ICD.P2A.HCV | PEACRPAAGGAVHTRGLDFACDIYIWAPLAGTCGV | |
| protease) | LLLSLVITLYCKSRQTPPPGGPGECTTPCSGSWLP | |
| GM-CSF leader sequence | GGSALLYRHWIEIVLLYRTYQSKDQTLGDKKDFDA | |
| HA Tag | FVSYAKWSSFPSEATSSLSEEHLALSLFPDVLENKY | |
| Disulfide linker | GYSLCLLERDVAPGGVYAEDIVSIIKRSRRGIFILS | |
| EE leucine zipper | PNYVNGPSIFELQAAVNLALDDQTLKLILIKFCYFQ | |
| CD8α | EPESLPHLVKKALRVLPTVTWRGLKSVPPNSRFWAK | |
| hinge | MRYHMPVKNSQGFTWNQLRITSRIFQWKGLSRTE | |
| CD8α transmembrane | TTGRSSQPKEWGSTATNFSLLKQAGDVEENPGPG | |
| domain | STMGTSLLCWMALCLLGADHADACPYSNPSLCSG | |
| IL15Rα transition peptide | GGGSELPTQGTFSNVSTNVSPAKPTTTACPYSNPS | |
| Linker | LCSGGGGSPAPRPPTPAPTIASQPLSLRPEACRPA | |
| HCV 4b5a cleavage site | AGGAVHTRGLDFACDIYIWAPLAGTCGVLLLSLVIT | |
| Linker | LYCNHRNRRRVCKCPRPVVGSSGNSSGGSTGCVV | |
| IL18RB | IVGRIVLSGSGTSAPITAYAQQTRGLLGCIITSLTG | |
| Linker | RDKNQVEGEVQIMSTATQTFLATCINGVCWTVYHG | |
| P2A | AGTRTIASPKGPVIQMYTNVDQDLVGWPAPQGSR | |
| Linker | SLTPCTCGSSDLYLVTRHADVIPVRRRGDGRGSLL | |
| TCRβ Leader | SPRPISYLKGSSGGPLLCPAGHAVGLFRAAVCTRG | |
| RQR8 Tag | VAKAVDFIPVENLETTMRSPVFTDNSSPPAVTLTHN | |
| CD8α Hinge | AS | |
| CD8α Tm | | |
| CD8α intracellular linker | | |
| NS4A cofactor domain | | |
| Linker | | |
| HCV NS3 protease | | |
| NS3 helicase fragment | | |
| Linker | | |
| 4a4b HCV NS3 protease cleavage | DEMEECSQHL | 28 |
| site | | |
| 4b5a HCV NS3 protease cleavage | ECTTPCSGSWL | 29 |
| site | | |
| 5a5b HCV NS3 protease cleavage | EDVVPCSMGS | 30 |
| site | | |

The present disclosure provides each of the polypeptides provided in Table 1, and each of the individual domains therein, as well as nucleic acids that encode such polypeptides and individual domains. Cells comprising such polypeptides and nucleic acids are also provided. As will be appreciated, the present disclosure also provides variants of any of the polypeptides and individual domains therein, where in some instances a variant polypeptide or domain thereof comprises an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, or 99% or greater amino acid sequence identity to the parental/reference sequence, or a functional fragment thereof, where the variant retains the functionality (e.g., the ability to dimerize, intracellular signaling activity, protease activity, cleavability by a protease, transition domain, and/or the like) of the parental/reference sequence.

For example, in certain embodiments, variants of such polypeptides having one or more amino acid substitutions are provided. Conservative substitutions are shown in Table 2 under the heading of “preferred substitutions.” More substantial changes are provided in Table 2 under the heading of “exemplary substitutions,” and as further described below in reference to amino acid side chain classes. Amino acid substitutions may be introduced into a polypeptide of interest and the products screened for a desired activity, e.g., retained/improved cell surface expression, dimerization, signaling, regulatability, and/or the like.

TABLE 2
Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gin (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

Example nucleotide sequences encoding the various domains and polypeptides set forth in Table 1 are provided in SEQ ID NOs: 31 to 53. Also encompassed by the present disclosure are nucleic acids encoding a polypeptide or domain thereof, which differ from a nucleotide sequence in SEQ ID NOS: 31 to 53 by virtue of the degeneracy of the genetic code.

In addition to the one or more nucleic acids and CCRs of the present disclosure described herein, aspects of the present disclosure further include membrane associated polypeptides and nucleic acids encoding the same. In some instances, provided are nucleic acids encoding a membrane associated polypeptide comprising a transmembrane domain and a cytokine receptor ICD, wherein the polypeptide does not comprise an extracellular cytokine binding domain. Such polypeptides are based in part on the unexpected findings herein that a single cytokine chain (i.e., not paired with a second cytokine chain) can enhance anti-tumor efficacy of CAR T cells as compared to CAR T cells lacking the single cytokine chain (see, e.g., Example 11). In certain embodiments, the cytokine receptor ICD is an IL-2Rβ ICD, an IL-7R ICD, an IL-9R ICD, or a common gamma chain (γc) ICD. According to some embodiments, the cytokine receptor ICD is an IL-2Rβ ICD. In some instances, the polypeptide comprises a heterologous dimerization domain, e.g., an extracellular heterologous dimerization domain. In certain embodiments, the polypeptide comprises a protease cleavage site disposed between the transmembrane domain and the ICD.

### Expression Constructs, Cells and Compositions

### Expression Constructs

Aspects of the present disclosure further include one or more expression constructs comprising any of the one or more nucleic acids of the present disclosure. As used herein, an “expression construct” is a circular or linear polynucleotide (a polymer composed of naturally occurring and/or non-naturally occurring nucleotides) comprising a region that encodes the first subunit, the second subunit, or both, of a CCR of the present disclosure, operably linked to a suitable promoter, e.g., a constitutive or inducible promoter. In some embodiments, expression of the first subunit, the second subunit, or both, is under the control of one or more exogenous (including heterologous) regulatory elements, e.g., promoter, enhancer, etc., present in the expression construct. In some embodiments, expression of the first subunit, the second subunit, or both, may be controlled by one or more endogenous regulatory elements, e.g., promoter, enhancer, etc., at or near a genomic locus into which the expression construct is inserted.

In certain embodiments, a promoter may be a single shared promoter among each of the protein-encoding regions of the expression construct, or at least one of the protein-encoding regions may be operably linked to a promoter which is not shared with any other protein-encoding region of the expression construct. An expression construct may be configured to allow for polycistronic expression of two or more (e.g., each) of the protein-encoding regions. That is, two or more (e.g., each) of the proteins encoded by the expression construct may be expressed as separate proteins from the same promoter. In certain embodiments, the expression construct includes a ribosome skipping site to allow for polycistronic expression of two or more (e.g., each) of the protein-encoding regions. A non-limiting example of a suitable ribosome skipping site which may be incorporated into expression constructs is the P2A ribosome skipping site from porcine teschovirus.

The expression constructs (e.g., vectors) can be suitable for replication and integration in prokaryotes, eukaryotes, or both. The expression constructs may contain functionally appropriately oriented transcription and translation terminators, initiation sequences, and promoters useful for regulation of the expression of the nucleic acid encoding the first subunit, the second subunit, or both. The expression constructs optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

To obtain high levels of expression of a cloned nucleic acid it is common to construct expression constructs which typically contain a strong promoter to direct transcription, a ribosome binding site for translational initiation, and a transcription/translation terminator, each in functional orientation to each other and to the protein-encoding sequence. Examples of regulatory regions suitable for this purpose in E. coli are the promoter and operator region of the E. coli tryptophan biosynthetic pathway, the leftward promoter of phage lambda (P_{L}), and the L-arabinose (araBAD) operon. The inclusion of selection markers in DNA vectors transformed in E. coli is also useful. Examples of such markers include genes specifying resistance to ampicillin, tetracycline, or chloramphenicol. Expression systems for expressing the first subunit, the second subunit, or both, of a CCR are available using, for example, E. coli, Bacillus sp. and Salmonella. E. coli systems may also be used. Transducing cells with nucleic acids (e.g., expression constructs) can involve, for example, incubating lipidic microparticles containing nucleic acids with cells or incubating viral vectors containing nucleic acids with cells within the host range of the vector.

In certain embodiments, upon delivery of one or more expression constructs to cells, one or more of the expression constructs are episomal (e.g., extra-chromosomal), where by “episome” or “episomal” is meant a polynucleotide that replicates independently of the cell's chromosomal DNA. A non-limiting example of an episome that may be employed is a plasmid.

According to some embodiments, upon delivery of one or more expression constructs to cells, one or more of the expression constructs integrates into the genome of the cell. In certain embodiments, one or more of the expression constructs are adapted for site-specific integration into the genome. For example, an expression construct may be adapted for site-specific integration into the genome, where the site-specific integration inactivates a target gene within the genome of the cell. By way of example, the site-specific integration may knock-out the target gene by knock-in of the expression construct. Any suitable approach for site-specific gene editing and functional integration may be employed. Functional integration of an expression construct may be achieved through various means, including through the use of integrating vectors, including viral and non-viral vectors. In some instances, a retroviral vector, e.g., a lentiviral vector, may be employed. In some instances, a non-retroviral integrating vector may be employed. An integrating vector may be contacted with the cells in a suitable transduction medium, at a suitable concentration (or multiplicity of infection), and for a suitable time for the vector to infect the target cells, facilitating functional integration of the expression construct. Non-limiting examples of useful viral vectors include retroviral vectors, lentiviral vectors, adenoviral (Ad) vectors, adeno-associated virus (AAV) vectors, hybrid Ad-AAV vector systems, and the like.

Strategies for site-specific integration that find use in the methods of the present disclosure include those that employ homologous recombination, nonhomologous end-joining (NHEJ), and/or the like. Such strategies may employ a non-naturally occurring or engineered nuclease, including, but not limited to, zinc-ringer nucleases (ZNFs), meganucleases, transcription activator-like effector nucleases (TALENs)), or a CRISPR-Cas system. Eukaryotic cells utilize two distinct DNA repair mechanisms in response to DNA double strand breaks (DSBs): Homologous recombination (HR) and nonhomologous end-joining (NHEJ). Mechanistically, HR is an error-free DNA repair mechanism because it requires a homologous template to repair the damaged DNA strand. Because of its homology-based mechanism, HR has been used as a tool to site-specifically engineer the genome. Gene targeting by HR requires the use of two homology arms that flank the transgene/target site of interest. HR efficiency can be increased by the introduction of DSBs at the target site using specific rare-cutting endonucleases. The discovery of this phenomenon prompted the development of methods to create site-specific DSBs in the genome of different species. Various chimeric enzymes have been designed for this purpose over the last decade, namely ZFNs, meganucleases, and TALENs. ZFNs are modular chimeric proteins that contain a ZF-based DNA binding domain (DBD) and a Fokl nuclease domain. DBD is usually composed of three ZF domains, each with 3-base pair specificity; the Fokl nuclease domain provides a DNA nicking activity, which is targeted by two flanking ZFNs. Owing to the modular nature of the DBD, any site in a genome could be targeted. TALENs are similar to ZFNs except that the DBD is derived from transcription activator-like effectors (TALEs). The TALE DBD is modular, and it is composed of 34-residue repeats, and its DNA specificity is determined by the number and order of repeats. Each repeat binds a single nucleotide in the target sequence through only two residues.

### Cells

Aspects of the present disclosure further include cells comprising the one or more nucleic acids of the present disclosure, as well as cells comprising the one or more expression constructs of the present disclosure. In certain embodiments, the cells are prokaryotic cells (e.g., bacteria), a yeast cells, insect (e.g., drosophila) cells, amphibian (e.g., frog, e.g., Xenopus) cells, plant cells, etc. According to some embodiments, the cells are mammalian cells. Mammalian cells of interest include human cells, rodent cells, and the like.

In some embodiments, the cells are immune cells. Non-limiting examples of immune cells include T cells, B cells, natural killer (NK) cells, macrophages, monocytes, neutrophils, dendritic cells, mast cells, basophils, and eosinophils. In certain embodiments, the cells are T cells. When the immune cells comprise T cells, the T cells may comprise one or any combination of naive T cells (T_{N}), cytotoxic T cells (T_{CTL}), memory T cells (T_{MEM}), T memory stem cells (T_{SCM}), central memory T cells (T_{CM}), effector memory T cells (T_{EM}), tissue resident memory T cells (T_{RM}), effector T cells (T_{EFF}), regulatory T cells (T_{REGs}), helper T cells, CD4+ T cells, CD8+ T cells, virus-specific T cells, alpha beta T cells (T_{αβ}), gamma delta T cells (T_{γδ}).

According to some embodiments, the cells are stem cells, e.g., mammalian (e.g., human) stem cells. For example, the population of cells may comprise embryonic stem (ES) cells, adult stem cells, hematopoietic stem cells (HSCs), induced pluripotent stem cells (iPSCs), mesenchymal stem cells (MSCs), neural stem cells (NSCs), or any combination thereof.

Approaches for introducing the one or more nucleic acids, or one or more expression constructs, into cells of interest are known and may include contacting a population of cells with the one or more nucleic acids, or one or more expression constructs, under conditions in which the one or more nucleic acids, or one or more expression constructs, are delivered to cells of the population of cells. The methods may further comprise selecting for cells exhibiting cell surface expression of the CCR.

The contacting step may comprise contacting the population of cells with the one or more nucleic acids, or one or more expression constructs, e.g., by combining the cells and the one or more nucleic acids, or one or more expression constructs, in a single mixture under conditions suitable for delivery (e.g., transfection, transduction, etc.) of each of the one or more nucleic acids, or one or more expression constructs, into cells of the population of cells.

A variety of suitable approaches and conditions for the delivery of nucleic acids and expression constructs to cells are known. According to some embodiments, delivery is carried out by microinjection, transfection, lipofection, heat-shock, electroporation, transduction, gene gun, DEAE-dextran-mediated transfer, and/or the like. In certain embodiments, the one or more nucleic acids, or one or more expression constructs, are introduced into cells of the population of cells by AAV transduction. The AAV vector may comprise ITRs from AAV2, and a serotype from any one of AAV1, AAV2, AAV3, AAV4, AAV5, AAV6, AAV7, AAV8, AAV9, or AAV 10. According to some embodiments, the AAV vector comprises ITRs from AAV2 and a serotype from AAV6. In certain embodiments, the one or more nucleic acids, or one or more expression constructs, are introduced into the cells (e.g., T cells) by lentiviral or retroviral transduction. The lentiviral vector backbone may be derived from HIV-1, HIV-2, visna-maedi virus (VMV) virus, caprine arthritis-encephalitis virus (CAEV), equine infectious anemia virus (EIAV), feline immunodeficiency virus (FIV), bovine immune deficiency virus (BIV), or simian immunodeficiency virus (SIV). The lentiviral vector may be integration competent or an integrase deficient lentiviral vector (TDLV). In one embodiment, IDLV vectors including an HIV-based vector backbone (i.e., HIV cis-acting sequence elements) are employed. Non-limiting example approaches for the preparation of retroviral expression constructs and the transduction of cells with such constructs is provided in the Experimental section hereinbelow.

In certain embodiments, the cells of the present disclosure (e.g., T cells, NK cells, etc.) are genetically modified to express a receptor (e.g., a recombinant receptor) on the surface thereof. That is, the cells may be engineered co-express the CCR and an additional receptor, non-limiting examples of which include a chimeric antigen receptor (CAR), a T cell receptor (TCR) such as a recombinant TCR, a synthetic notch receptor (synNotch), a Modular Extracellular Sensor Architecture (MESA) receptor, a Tango receptor, a ChaCha receptor, a generalized extracellular molecule sensor (GEMS) receptor, a growth factor receptor, a switch receptor, an adhesion molecule, an integrin, an inhibitory receptor, a stimulatory receptor, an immunoreceptor tyrosine-based activation motif (ITAM)-containing receptor, an immunoreceptor tyrosine-based inhibition motif (ITIM)-containing receptor, a hormone receptor, a receptor tyrosine kinase, an immune receptor such as CD28, CD80, ICOS, CTLA4, PD1, PD-L1, BTLA, HVEM, CD27, 4-1BB, 4-1BBL, OX40, OX40L, DR3, GITR, CD30, SLAM, CD2, 2B4, TIM1, TIM2, TIM3, TIGIT, CD226, CD160, LAG3, LAIR1, B7-1, B7-H1, and B7-H3, a type I cytokine receptor such as Interleukin-1 receptor, Interleukin-2 receptor, Interleukin-3 receptor, Interleukin-4 receptor, Interleukin-5 receptor, Interleukin-6 receptor, Interleukin-7 receptor, Interleukin-9 receptor, Interleukin-11 receptor, Interleukin-12 receptor, Interleukin-13 receptor, Interleukin-15 receptor, Interleukin-18 receptor, Interleukin-21 receptor, Interleukin-23 receptor, Interleukin-27 receptor, Erythropoietin receptor, GM-CSF receptor, G-CSF receptor, Growth hormone receptor, Prolactin receptor, Leptin receptor, Oncostatin M receptor, Leukemia inhibitory factor, a type II cytokine receptor such as interferon-alpha/beta receptor, interferon-gamma receptor, Interferon type III receptor, Interleukin-10 receptor, Interleukin-20 receptor, Interleukin-22 receptor, Interleukin-28 receptor, a receptor in the tumor necrosis factor receptor superfamily such as Tumor necrosis factor receptor 2 (1B), Tumor necrosis factor receptor 1, Lymphotoxin beta receptor, OX40, CD40, Fas receptor, Decoy receptor 3, CD27, CD30, 4-1BB, Decoy receptor 2, Decoy receptor 1, Death receptor 5, Death receptor 4, RANK, Osteoprotegerin, TWEAK receptor, TACI, BAFF receptor, Herpesvirus entry mediator, Nerve growth factor receptor, B-cell maturation antigen, Glucocorticoid-induced TNFR-related, TROY, Death receptor 6, Death receptor 3, Ectodysplasin A2 receptor, a chemokine receptor such as CCR1, CCR2, CCR3, CCR4, CCR5, CCR6, CCR7, CCR8, CCR9, CCR10, CXCR1, CXCR2, CXCR3, CXCR4, CXCR5, CXCR6, CX3CR1, XCR1, ACKR1, ACKR2, ACKR3, ACKR4, CCRL2, a receptor in the epidermal growth factor receptor (EGFR) family, a receptor in the fibroblast growth factor receptor (FGFR) family, a receptor in the vascular endothelial growth factor receptor (VEGFR) family, a receptor in the rearranged during transfection (RET) receptor family, a receptor in the Eph receptor family, a receptor that can induce cell differentiation (e.g., a Notch receptor), a cell adhesion molecule (CAM), an adhesion receptor such as integrin receptor, cadherin, selectin, and a receptor in the discoidin domain receptor (DDR) family, transforming growth factor beta receptor 1, and transforming growth factor beta receptor 2. In some embodiments, such a receptor is an immune cell receptor selected from a T cell receptor, a B cell receptor, a natural killer (NK) cell receptor, a macrophage receptor, a monocyte receptor, a neutrophil receptor, a dendritic cell receptor, a mast cell receptor, a basophil receptor, and an eosinophil receptor.

In certain embodiments, cells of the present disclosure are engineered to express a chimeric antigen receptor (CAR) in addition to the CCR. According to some embodiments, cells of the present disclosure are engineered to express a recombinant TCR in addition to the CCR.

As described above, according to some embodiments, the cells of the present disclosure (e.g., human T cells) may be engineered to express a CAR in addition to the CCR. The extracellular binding domain of the CAR may comprise a single chain antibody. The single-chain antibody may be a monoclonal single-chain antibody, a chimeric single-chain antibody, a humanized single-chain antibody, a fully human single-chain antibody, and/or the like. In one non-limiting example, the single chain antibody is a single chain variable fragment (scFv). In some embodiments, the extracellular binding domain of the CAR is a single-chain version (e.g., an scFv version) of an antibody approved by the United States Food and Drug Administration and/or the European Medicines Agency (EMA) for use as a therapeutic antibody. Non-limiting examples of single-chain antibodies which may be employed when the protein of interest is a CAR include single-chain versions (e.g., scFv versions) of Adecatumumab, Ascrinvacumab, Cixutumumab, Conatumumab, Daratumumab, Drozitumab, Duligotumab, Durvalumab, Dusigitumab, Enfortumab, Enoticumab, Figitumumab, Ganitumab, Glembatumumab, Intetumumab, Ipilimumab, Iratumumab, Icrucumab, Lexatumumab, Lucatumumab, Mapatumumab, Narnatumab, Necitumumab, Nesvacumab, Ofatumumab, Olaratumab, Panitumumab, Patritumab, Pritumumab, Radretumab, Ramucirumab, Rilotumumab, Robatumumab, Seribantumab, Tarextumab, Teprotumumab, Tovetumab, Vantictumab, Vesencumab, Votumumab, Zalutumumab, Flanvotumab, Altumomab, Anatumomab, Arcitumomab, Bectumomab, Blinatumomab, Detumomab, Ibritumomab, Minretumomab, Mitumomab, Moxetumomab, Naptumomab, Nofetumomab, Pemtumomab, Pintumomab, Racotumomab, Satumomab, Solitomab, Taplitumomab, Tenatumomab, Tositumomab, Tremelimumab, Abagovomab, Igovomab, Oregovomab, Capromab, Edrecolomab, Nacolomab, Amatuximab, Bavituximab, Brentuximab, Cetuximab, Derlotuximab, Dinutuximab, Ensituximab, Futuximab, Girentuximab, Indatuximab, Isatuximab, Margetuximab, Rituximab, Siltuximab, Ublituximab, Ecromeximab, Abituzumab, Alemtuzumab, Bevacizumab, Bivatuzumab, Brontictuzumab, Cantuzumab, Cantuzumab, Citatuzumab, Clivatuzumab, Dacetuzumab, Demcizumab, Dalotuzumab, Denintuzumab, Elotuzumab, Emactuzumab, Emibetuzumab, Enoblituzumab, Etaracizumab, Farletuzumab, Ficlatuzumab, Gemtuzumab, Imgatuzumab, Inotuzumab, Labetuzumab, Lifastuzumab, Lintuzumab, Lorvotuzumab, Lumretuzumab, Matuzumab, Milatuzumab, Nimotuzumab, Obinutuzumab, Ocaratuzumab, Otlertuzumab, Onartuzumab, Oportuzumab, Parsatuzumab, Pertuzumab, Pinatuzumab, Polatuzumab, Sibrotuzumab, Simtuzumab, Tacatuzumab, Tigatuzumab, Trastuzumab, Tucotuzumab, Vandortuzumab, Vanucizumab, Veltuzumab, Vorsetuzumab, Sofituzumab, Catumaxomab, Ertumaxomab, Depatuxizumab, Ontuxizumab, Blontuvetmab, Tamtuvetmab, or an antigen-binding variant thereof.

The additional receptor (e.g., CAR) may include one or more linker sequences between the various domains. A “variable region linking sequence” is an amino acid sequence that connects a heavy chain variable region to a light chain variable region and provides a spacer function compatible with interaction of the two sub-binding domains so that the resulting polypeptide retains a specific binding affinity to the same target molecule as an antibody that includes the same light and heavy chain variable regions. A non-limiting example of a variable region linking sequence is a glycine-serine linker, such as a (G4S) 3 (SEQ ID NO:63) linker. In certain embodiments, a linker separates one or more heavy or light chain variable domains, hinge domains, transmembrane domains, co-stimulatory domains, and/or primary signaling domains. In particular embodiments, the receptor (e.g., CAR) includes one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids in length.

In some embodiments, the antigen binding domain of the receptor (e.g., CAR) is followed by one or more spacer domains that moves the antigen binding domain away from the cell surface expressing the receptor to enable proper cell/cell contact, antigen binding and/or activation. The spacer domain (and any other spacer domains, linkers, and/or the like described herein) may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain may include the amino acid sequence of a naturally occurring immunoglobulin hinge region or an altered immunoglobulin hinge region. In some embodiments, the spacer domain includes the CH2 and/or CH3 of IgG1, IgG4, or IgD. Illustrative spacer domains suitable for use in the receptors (e.g., CARs) described herein include the hinge region derived from the extracellular regions of type 1 membrane proteins such as CD8a and CD4, which may be wild-type hinge regions from these molecules or variants thereof. In certain embodiments, the hinge domain includes a CD8a hinge region. According to some embodiments, the hinge is a PD-1 hinge or CD152 hinge. In certain embodiments, the hinge is an IgG4 hinge.

The “transmembrane domain” (Tm domain) is the portion of the receptor (e.g., CAR) that fuses the extracellular binding portion and intracellular signaling domain and anchors the receptor to the plasma membrane of the cell (e.g., T-cell, such as a Treg). The Tm domain may be derived either from a natural, synthetic, semi-synthetic, or recombinant source. In some embodiments, the Tm domain is derived from (e.g., includes at least the transmembrane region(s) or a functional portion thereof) of the alpha or beta chain of the T-cell receptor, CD35, CD37, CD3γ, CD30, CD4, CD5, CD8a, CD9, CD16, CD22, CD27, CD28, CD33, CD37, CD45, CD64, CD80, CD86, CD134, CD137, CD152, CD154, or PD-1.

In one embodiment, a receptor (e.g., CAR) includes a Tm domain derived from CD28. In certain embodiments, a receptor includes a Tm domain derived from CD28 and a short oligo- or polypeptide linker, e.g., between 1, 2, 3, 4, 5, 6, 7, 8, 9, or 10 amino acids in length, that links the Tm domain and the intracellular signaling domain of the receptor. A glycine-serine linker may be employed as such a linker, for example.

The “intracellular signaling” domain of a receptor (e.g., a CAR) refers to the part of the receptor that participates in transducing the signal from binding to a target molecule/antigen into the interior of the cell to elicit cell function. Accordingly, the term “intracellular signaling domain” refers to the portion of a protein which transduces the signal and that directs the cell to perform a specialized function. To the extent that a truncated portion of an intracellular signaling domain is used, such truncated portion may be used in place of a full-length intracellular signaling domain as long as it transduces the signal. The term intracellular signaling domain is meant to include any truncated portion of an intracellular signaling domain sufficient for transducing signal.

Signals generated through the T cell receptor (TCR) alone are insufficient for full activation of the T cell, and a secondary or costimulatory signal is also required. Thus, T cell activation is mediated by two distinct classes of intracellular signaling domains: primary signaling domains that initiate antigen-dependent primary activation through the TCR (e.g., a TCR/CD3 complex) and costimulatory signaling domains that act in an antigen-independent manner to provide a secondary or costimulatory signal. As such, a receptor (e.g., CAR) expressed by a genetically modified cell may include an intracellular signaling domain that includes one or more (e.g., 1, 2, or more) “costimulatory signaling domains” and a “primary signaling domain.”

Primary signaling domains regulate primary activation of the TCR complex either in a stimulatory manner, or in an inhibitory manner. Primary signaling domains that act in a stimulatory manner may contain signaling motifs which are known as immunoreceptor tyrosine-based activation motifs (or “ITAMs”). Non-limiting examples of ITAM-containing primary signaling domains suitable for use in a receptor of the present disclosure include those derived from FcRγ, FcRβ, CD3γ, CD30, CD38, CD3Z, CD22, CD79a, CD79B, and CD660. In certain embodiments, a receptor includes a CD34 primary signaling domain and one or more costimulatory signaling domains. The intracellular primary signaling and costimulatory signaling domains are operably linked to the carboxyl terminus of the transmembrane domain.

In some embodiments, the receptor (e.g., CAR) includes one or more costimulatory signaling domains to enhance the efficacy and expansion of immune effector cells (e.g., T cells) expressing the receptor. As used herein, the term “costimulatory signaling domain” or “costimulatory domain” refers to an intracellular signaling domain of a costimulatory molecule or an active fragment thereof. Example costimulatory molecules suitable for use in receptors contemplated in particular embodiments include TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, KD2C, SLP76, TRIM, and ZAP70. In some embodiments, the receptor (e.g., CAR) includes one or more costimulatory signaling domains selected from the group consisting of 4-1BB (CD137), CD28, and CD134, and a CD3ζ primary signaling domain.

A receptor (e.g., CAR) expressed by a cell genetically modified according to the methods of the present disclosure may include any variety of suitable domains including but not limited to a leader sequence; hinge, spacer and/or linker domain(s); transmembrane domain(s); costimulatory domain(s); signaling domain(s) (e.g., CD3ζ domain(s)); ribosomal skip element(s); restriction enzyme sequence(s); reporter protein domains; and/or the like.

According to some embodiments, the extracellular binding domain of the receptor (e.g., a CAR) specifically binds a tumor antigen expressed on the surface of a cancer cell. Non-limiting examples of tumor antigens to which the extracellular binding domain of the receptor may specifically bind include 5T4, AXL receptor tyrosine kinase (AXL), B-cell maturation antigen (BCMA), c-MET, C4.4a, carbonic anhydrase 6 (CA6), carbonic anhydrase 9 (CA9), Cadherin-6, CD19, CD20, CD22, CD25, CD27L, CD30, CD33, CD37, CD44, CD44v6, CD56, CD70, CD74, CD79b, CD123, CD138, carcinoembryonic antigen (CEA), cKit, Cripto protein, CS1, delta-like canonical Notch ligand 3 (DLL3), endothelin receptor type B (EDNRB), ephrin A4 (EFNA4), epidermal growth factor receptor (EGFR), EGFRvIII, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), GD2 ganglioside (“GD2”), glycoprotein non-metastatic B (GPNMB), guanylate cyclase 2 C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), programmed cell death receptor ligand 1 (PD-L1), programmed cell death receptor ligand 2 (PD-L2), prostate-specific membrane antigen (PSMA), protein tyrosine kinase 7 (PTK7), solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), Tn antigen, trophoblast cell-surface antigen (TROP-2), Wilms' tumor 1 (WT1), and VEGF-A.

In certain embodiments, the cells of the present disclosure are genetically modified to express an antibody. The term “antibody” (also used interchangeably with “immunoglobulin”) encompasses antibodies of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the antigen, including, but not limited to single chain Fv (scFv), Fab, (Fab′)₂, (scFv′)₂, and diabodies; chimeric antibodies; monoclonal antibodies, humanized antibodies, human antibodies; and fusion proteins comprising an antigen-binding portion of an antibody and a non-antibody protein.

Immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (usually of about 25 kDa or about 214 amino acids) comprise a variable region of about 110 amino acids at the NH₂-terminus and a kappa or lambda constant region at the COOH-terminus. Full-length immunoglobulin “heavy chains” (of about 150 kDa or about 446 amino acids), similarly comprise a variable region (of about 116 amino acids) and one of the aforementioned heavy chain constant regions, e.g., gamma (of about 330 amino acids).

An immunoglobulin light or heavy chain variable region (V_{L} and V_{H}, respectively) is composed of a “framework” region (FR) interrupted by three hypervariable regions, also called “complementarity determining regions” or “CDRs”. The extent of the framework region and CDRs have been defined (see, E. Kabat et al., Sequences of proteins of immunological interest, 4th ed. U.S. Dept. Health and Human Services, Public Health Services, Bethesda, MD (1987); and Lefranc et al. IMGT, the international ImMunoGeneTics information System®. Nucl. Acids Res., 2005, 33, D593-D597)). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen. All CDRs and framework provided by the present disclosure are defined according to Kabat, supra, unless otherwise indicated.

An “antibody” thus encompasses a protein having one or more polypeptides that can be genetically encodable, e.g., by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. In some embodiments, an antibody of the present disclosure is an IgG antibody, e.g., an IgG1 antibody, such as a human IgG1 antibody. In some embodiments, the cell expresses an antibody that comprises a human Fc domain.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

According to some embodiments, the cells of the present disclosure are not genetically modified to express an engineered receptor other than the CCR.

As will be appreciated with the benefit of the present disclosure, the cells of the present disclosure (e.g., T cells) may be expanded during a therapeutic cell manufacturing process. By “expanding” or “expanded” is meant the cells are cultured under conditions in which the cells proliferate. Suitable conditions may vary depending upon, e.g., the type of cells (e.g., the type of T cells) being expanded. Such conditions may include culturing the cells (e.g., T cells) in a suitable container (e.g., a cell culture plate or well thereof, a cassette, tube, bottle or bag suitable for use in an automated therapeutic cell manufacturing system, e.g., a closed automated therapeutic cell manufacturing system such as the CliniMACS Prodigy® system by Miltenyi Biotec, the Xuri® cell expansion system by Cytiva, the G-Rex® cell expansion system by Wilson Wolf, the Quantum® cell expansion system from Terumo, the Cocoon® system by Lonza, or the like), in suitable medium (e.g., cell culture medium, such as RPMI, DMEM, IMDM, MEM, DMEM/F-12, or the like) at a suitable temperature (e.g., 32° C.-42° C., such as 37° C.) and pH (e.g., pH 7.0-7.7, such as pH 7.4) in an environment having a suitable percentage of CO₂, e.g., 3% to 10%, such as 5%.

Methods for activating and expanding cells for therapy (e.g., therapeutic T cells and the like) are known in the art and are described, e.g., in U.S. Pat. Nos. 6,905,874; 6,867,041; and 6,797,514; and PCT Publication No. WO 2012/079000, the contents of which are hereby incorporated by reference in their entirety. In the example of T cells, such methods may include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is the Dynabeads® system, a CD3/CD28 activator/stimulator system for physiological activation of human T cells. In other embodiments, the T cells are activated and stimulated to proliferate with feeder cells and appropriate antibodies and cytokines using methods such as those described in U.S. Pat. Nos. 6,040,177 and 5,827,642 and PCT Publication No. WO 2012/129514, the contents of which are hereby incorporated by reference in their entirety.

In certain embodiments, cells of the present disclosure are expanded using an automated system designed for the manufacture of therapeutic cells. Non-limiting examples of such systems include the CliniMACS Prodigy® system by Miltenyi Biotec, the Xuri® cell expansion system by Cytiva, the G-Rex® cell expansion system by Wilson Wolf, the Quantum® cell expansion system from Terumo, the Cocoon® system by Lonza, etc. Detailed guidance and protocols for manufacturing therapeutic cells on such systems are available from the providers of such systems.

### Compositions

Also provided by the present disclosure are compositions. According to some embodiments, provided are compositions comprising any of the cells of the one or more nucleic acids, one or more expression constructs, and/or cells of the present disclosure or progeny thereof.

Such compositions may comprise the one or more nucleic acids, one or more expression constructs, and/or cells of the present disclosure present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl) piperazine-N′-(2-ethanesulfonic acid) (HEPES), 2-(N-Morpholino) ethanesulfonic acid (MES), 2-(N-Morpholino) ethanesulfonic acid sodium salt (MES), 3-(N-Morpholino) propanesulfonic acid (MOPS), N-tris [Hydroxymethyl]methyl-3-aminopropanesulfonic acid (TAPS), etc.), a solubilizing agent, a detergent (e.g., a non-ionic detergent such as Tween-20, etc.), a nuclease inhibitor, glycerol, a chelating agent, and the like may be present in such compositions. In certain embodiments, the liquid medium is a cell culture medium. Non-limiting examples of cell culture media include Minimal Essential Media, DMEM, a-MEM, RPMI Media, Clicks, F-12, X-Vivo 15, X-Vivo 20, Optimizer, and the like.

In certain embodiments, provided are compositions comprising the cells of the present disclosure, wherein the compositions are suitable for administration to a subject, e.g., a human subject. Such compositions may comprise the cells and a pharmaceutically acceptable carrier. The compositions generally include a therapeutically effective amount of the cells. By “therapeutically effective amount” is meant a number of cells sufficient to produce a desired result, e.g., an amount sufficient to effect beneficial or desired therapeutic (including preventative) results, such as a reduction in a symptom of a disease (e.g., cancer) or disorder associated, e.g., with a target cell or a population thereof (e.g., cancer cells), as compared to a control. An effective amount can be administered in one or more administrations. A therapeutically effective amount may vary according to factors such as the disease state, age, sex, and weight of the individual, and the ability of the cells to elicit a desired response in the individual. A therapeutically effective amount is also one in which any toxic or detrimental effects of the cells are outweighed by the therapeutically beneficial effects. The term “therapeutically effective amount” includes an amount that is effective to “treat” a subject, e.g., a human subject. When a therapeutic amount is indicated, the precise amount of the compositions contemplated in particular embodiments, to be administered, can be determined by a physician in view of the specification and with consideration of individual differences in age, weight, tumor size, extent of infection or metastasis, and condition of the patient (individual). In some embodiments, a composition of the present disclosure includes from 1×10⁶ to 5×10¹⁰ of the cells of the present disclosure.

The cells of the present disclosure can be incorporated into a variety of formulations for therapeutic administration. For example, the cells of the present disclosure can be formulated into pharmaceutical compositions by combination with appropriate, pharmaceutically acceptable excipients or diluents. Formulations of the cells suitable for administration to a patient (e.g., suitable for human administration) are generally sterile and may further be free of detectable pyrogens or other contaminants contraindicated for administration to a patient according to a selected route of administration.

The cells may be formulated for parenteral (e.g., intravenous, intra-arterial, intraosseous, intramuscular, intracerebral, intracerebroventricular, intrathecal, subcutaneous, etc.) administration, or any other suitable route of administration.

An aqueous formulation of the cells may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the formulation to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

In some embodiments, the composition comprises cells of the present disclosure, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/V).

### Kits

Also provided by the present disclosure are kits. In certain embodiments, provided are kits that include any of the one or more nucleic acids, or any of the one or more expression constructs, of the present disclosure. In certain embodiments, the kits of the present disclosure include transfection/transduction reagents useful for introducing the one or more nucleic acids, or any of the one or more expression constructs, into cells of interest, e.g., immune cells (e.g., T cells) or other cells of interest.

Components of the kits may be present in separate containers, or multiple components may be present in a single container. For example, the one or more nucleic acids, or any of the one or more expression constructs, may be provided in separate containers or the same container. A suitable container includes a single tube (e.g., vial), one or more wells of a plate (e.g., a 96-well plate, a 384-well plate, etc.), or the like.

The kits of the present disclosure may further comprise instructions for contacting a population of cells with the one or more nucleic acids, or any of the one or more expression constructs, under conditions in which the one or more nucleic acids, or any of the one or more expression constructs, are delivered to cells of the population of cells. The kits of the present disclosure may further comprise instructions for selecting for cells exhibiting cell surface expression of the CCR.

The instructions of the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging), etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, the means for obtaining the instructions is recorded on a suitable substrate.

### Therapeutic Methods

Aspects of the present disclosure further include therapeutic methods. As demonstrated herein, when expressed on cells, the CCRs of the present disclosure are strong drivers of cell proliferation in vitro and in vivo, and provide for enhanced in vivo anti-tumor activity as compared to control cells lacking the CCR. Accordingly, the CCRs and cells expressing the same find use in cell therapy, non-limiting examples of which include immune cell therapy (e.g., immune cells (e.g., tumor-infiltrating lymphocytes) lacking an antigen-binding receptor), CAR-T cell therapy, TCR therapy, CAR-NK cell therapy, and the like.

According to some embodiments, provided are methods of administering a cell-based therapy to a subject in need thereof, the method comprising administering an effective amount of a composition comprising cells of the present disclosure to the subject. The cells comprise any of the one more expression constructs of the present disclosure, such that the cells express the CCR comprising the first and second subunits encoded by the one or more nucleic acids. The cells may be further modified to express any of the additional receptors described herein, e.g., a CAR, a recombinant TCR, or the like.

A “cell based therapy” or “cell therapy” refers to the transfer of autologous or allogeneic cellular material into a subject for medical purposes. Non-limiting examples of cell-based therapies include CAR T cell therapy, engineered T cell therapy (e.g., T cells that express a recombinant T cell receptor (TCR)), a therapy comprising administering T cells which do not express a recombinant receptor other than the CCR, CAR NK cell therapy, and the like.

In certain embodiments, signaling of the chimeric cytokine receptor is regulatable. For example, in some embodiments: the first subunit, the second subunit, or both, comprises a protease cleavage site disposed between the transmembrane domain and the ICD; the one or more nucleic acids further encode a protease, wherein the protease cleavage site is a cleavage site for the protease; and proteolytic activity of the protease is regulatable.

According to some embodiments, proteolytic activity of the protease is regulatable via a cell-permeable small molecule. For example, the cell-permeable small molecule may be an inhibitor of the protease, and the method may further comprise administering an effective amount of the cell-permeable small molecule to the subject when signaling by the CCR is desired. In certain embodiments, such methods further comprise ceasing administration of the cell-permeable small molecule when signaling by the CCR is no longer desired.

When the cell-based therapy is regulatable using a cell-permeable protease inhibitor, the inhibitor of the protease is selected based on the protease employed in the regulatable system. For example, in some embodiments, when the protease is derived from HCV NS3, the inhibitor of the protease may be asunaprevir (ASV), danoprevir (DPV), simeprevir (SPV), grazoprevir (GPV), glecaprevir, voxilaprevir, or any combination thereof. Also by way of example, when the protease is an HIV protease, the inhibitor of the protease may be atazanavir (Reyataz), darunavir (Prezista), fosamprenavir (Lexiva), indinavir (Crixivan), lopinavir/ritonavir (Kaletra), nelfinavir (Viracept), ritonavir (Norvir), saquinavir (Invirase), tipranavir (Aptivus), atazanavir/cobicistat (Evotaz), darunavir/cobicistat (Prezcobix), or any combination thereof. As an additional example, when the protease is a SARS-CoV-2 protease, the inhibitor of the protease may be nirmatrelvir (Paxlovid). As a further example, when the protease is a human renin protease, the inhibitor of the protease may be aliskiren.

The therapeutic cells may be autologous/autogeneic (“self”) or non-autologous (“non-self,” e.g., allogeneic, syngeneic or xenogeneic). “Autologous” as used herein, refers to cells obtained from the subject to whom the therapeutic cells are later administered. “Allogeneic” as used herein refers to cells obtained from a donor other than the subject to whom the therapeutic cells are administered. In some embodiments, the cells (e.g., T cells) are cells obtained from a mammalian subject. In certain embodiments, the mammalian subject is a primate. In some embodiments, the cells are obtained from a human.

Any of the cell-based therapeutic methods of the present disclosure may be used to treat a variety of conditions in the subject. In certain embodiments, the subject has cancer. The terms “cancer” and “cancerous” refer to or describe the physiological condition in mammals that is typically characterized by unregulated cell growth/proliferation. The methods may be employed for the treatment of a large variety of cancers. In certain embodiments, the cancer comprises a solid tumor. Examples of solid tumors treatable by the methods of the present disclosure include carcinomas, lymphomas, blastomas, and sarcomas. In certain embodiments, the cancer comprises a hematological malignancy, non-limiting examples of which include a leukemia, a lymphoma, or multiple myeloma.

More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bile duct cancer, bladder cancer, hepatoma, breast cancer, colon cancer, endometrial or uterine carcinoma, salivary gland carcinoma, kidney cancer, prostate cancer, vulval cancer, thyroid cancer, hepatic carcinoma, various types of head and neck cancer, and the like. In certain embodiments, the individual has a cancer selected from a solid tumor, recurrent glioblastoma multiforme (GBM), non-small cell lung cancer, metastatic melanoma, melanoma, peritoneal cancer, epithelial ovarian cancer, glioblastoma multiforme (GBM), metastatic colorectal cancer, colorectal cancer, pancreatic ductal adenocarcinoma, squamous cell carcinoma, esophageal cancer, gastric cancer, neuroblastoma, fallopian tube cancer, bladder cancer, metastatic breast cancer, pancreatic cancer, soft tissue sarcoma, recurrent head and neck cancer squamous cell carcinoma, head and neck cancer, anaplastic astrocytoma, malignant pleural mesothelioma, squamous non-small cell lung cancer, rhabdomyosarcoma, metastatic renal cell carcinoma, basal cell carcinoma (basal cell epithelioma), and gliosarcoma. In certain aspects, the individual has a cancer selected from melanoma, Hodgkin lymphoma, renal cell carcinoma (RCC), bladder cancer, non-small cell lung cancer (NSCLC), and head and neck squamous cell carcinoma (HNSCC).

By treatment is meant at least an amelioration of one or more symptoms associated with the condition (e.g., cancer) of the subject, where amelioration is used in a broad sense to refer to at least a reduction in the magnitude of a parameter, e.g., symptom, associated with the condition being treated. As such, treatment also includes situations where the condition (e.g., cancer), or at least one or more symptoms associated therewith, are completely inhibited, e.g., prevented from happening, or stopped, e.g., terminated, such that the subject no longer suffers from the condition, or at least the symptoms that characterize the condition.

The following examples are offered by way of illustration and not by way of limitation.

### EXPERIMENTAL

### Example 1—Generation of Chimeric Cytokine Receptors (CCRs) that Signal in the Absence of their Cognate Cytokines

Described in this example is the generation of chimeric cytokine receptors (CCRs) capable of signaling in the absence of their cognate cytokines. The CCRs include a first subunit comprising a first heterologous dimerization domain and a first cytokine receptor intracellular signaling domain (ICD), and a second subunit comprising a second heterologous dimerization domain cognate for the first heterologous dimerization domain, and a second cytokine receptor ICD. As proof of concept, in this example, the CCRs employ: leucine zippers as the first and second heterologous dimerization domains; a first ICD which is IL-2RB or IL-21R; and a second ICD which is a common gamma chain (γC) ICD.

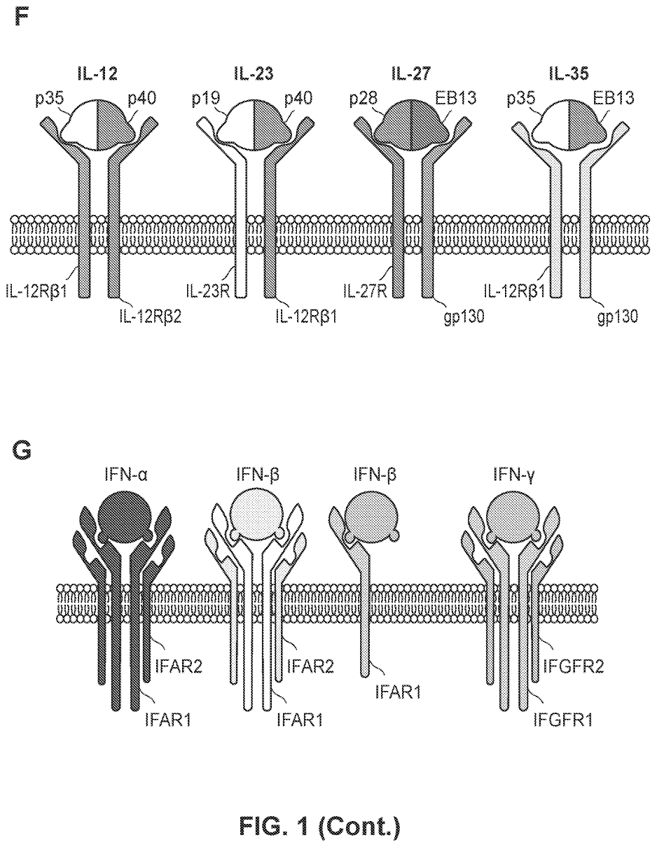

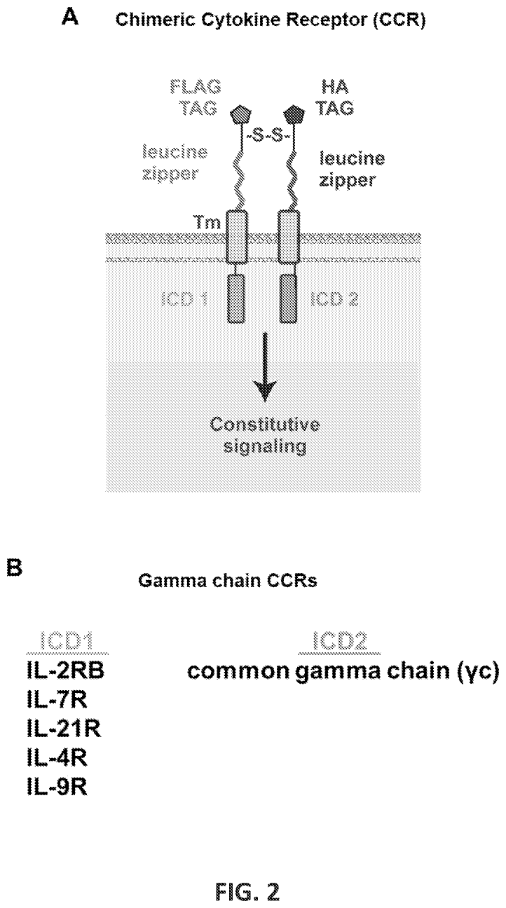

Shown in FIG. 2A is a schematic of the chimeric cytokine receptor (CCR) system, which allows for constitutive signaling of receptors in the absence of their cognate cytokines. Receptor 1 of the CCR is comprised of a first dimerization domain such as a leucine zipper fused to a transmembrane domain and a first intracellular domain (ICD 1). Receptor 2 of the CCR is comprised of a second dimerization domain which is cognate for the first dimerization domain such as a second leucine zipper fused to a transmembrane domain and a second intracellular domain (ICD 2). Association of the two dimerization domains results in close proximity of the two intracellular signaling domains and triggers signaling of the receptor complex. Cysteine residues are optionally included in the extracellular domain to stabilize the association of the receptor complex via disulfide bonds. Detection tags are optionally included to facilitate detection of cell surface expression of the receptor components. FIG. 2B shows example signaling domains for chimeric cytokine receptors based on gamma chain cytokine receptors.

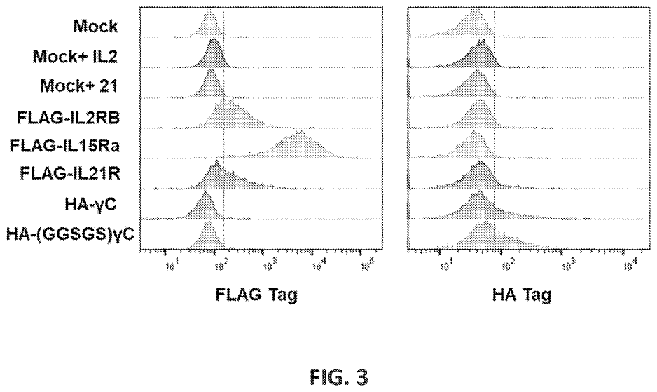

FIG. 3 is a series of flow plots demonstrating cell surface expression of the CCR receptor components for various common gamma chain receptors. The receptor components are comprised of an extracellular detection module (HA or Flag Tag), a leucine zipper domain, a CD8a hinge and transmembrane domain and an intracellular signaling domain (IL-2RB, IL-21R, or γC). As can be seen in the data, receptors containing a γC intracellular domain, with or without a GGSGS linker, displayed poor cell surface expression. Mock non-transduced T cells with or without added cytokines serve as negative controls for FLAG and HA Tag.

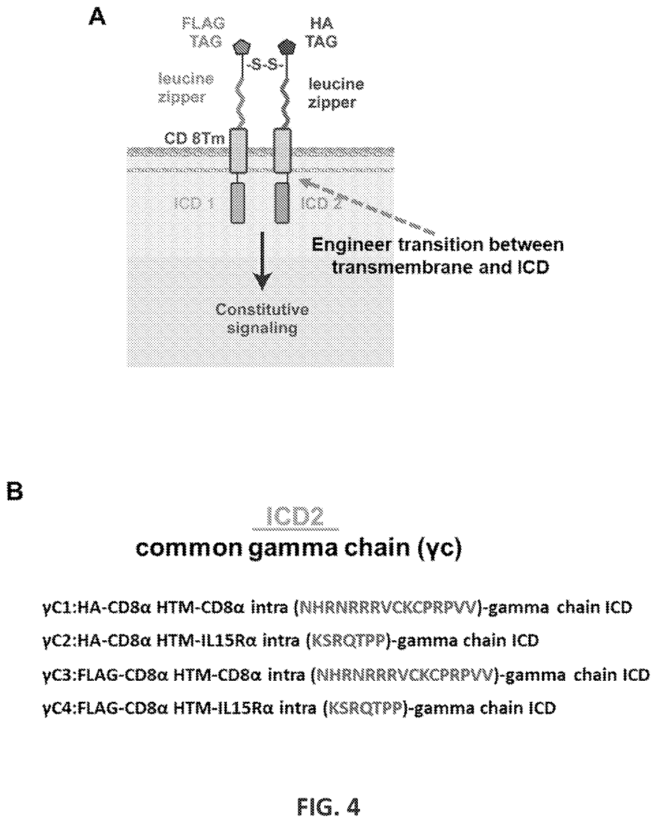

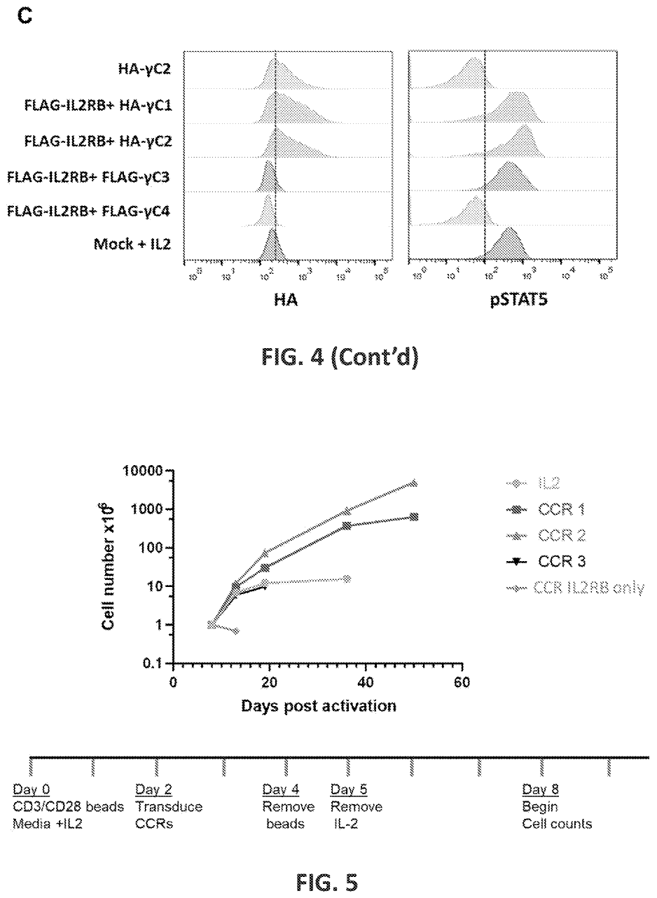

Shown in FIG. 4A is a schematic of the chimeric cytokine receptor (CCR) system. In order to improve expression of CCR receptors containing a common gamma chain intracellular domain, the transition between the transmembrane domain and the intracellular domain was engineered. FIG. 4B is a list of various CCR constructs containing a common gamma chain intracellular domain and a transition peptide between the transmembrane domain and the ICD domain. The transition peptides were derived from the juxtamembrane region of CD8 or IL-15Rα. HTM refers to hinge and transmembrane domain. FIG. 4C shows a series of flow plots for an IL-2R CCR demonstrating cell surface expression of the HA tag and phosphorylated STAT5 (pSTAT5), which is a cytokine signaling molecule downstream of IL-2R signaling. As can be seen in the data, inclusion of a transition peptide in the juxtamembrane domain improves cell surface expression of the common gamma chain CCR receptor component and allows for high levels of IL-2 signaling to be induced in the absence of cytokine. Mock non-transduced T cells exposed to exogenously added IL-2 serve as a positive control for pSTAT5.

Shown in FIG. 5 is a plot showing proliferation of primary human T cells transduced with the various CCRs shown in FIG. 4. As can be seen in the data the CCRs drive proliferation of T cells in the absence of added IL-2. Mock non-transduced T cells with added IL-2 serves as a positive control. T cells that were transduced with only one component of the CCR (CCR IL-2RB only) do not proliferate in the absence of IL-2). The CCRs drive T cell proliferation to various degrees and the magnitude of proliferation is correlated with the strength of pSTAT5 signaling shown in FIG. 4.

### Example 2—Development of a Regulatable CCR System that Addresses In Vivo Toxicity Observed for Constitutive CCRs

Shown in this example is that the constitutive CCRs described in Example 1 exhibit toxicity in vivo. However, demonstrated herein is the developments of a regulatable CCR system which addresses such toxicity.

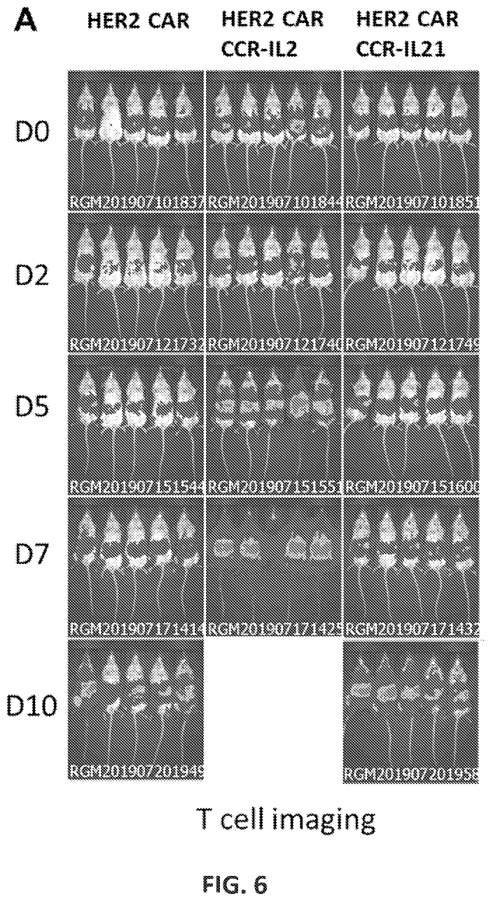

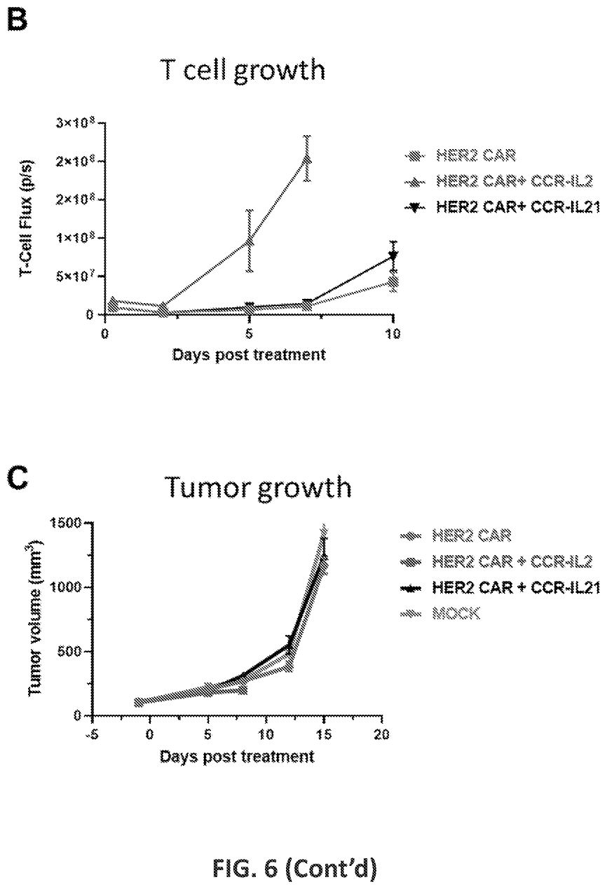

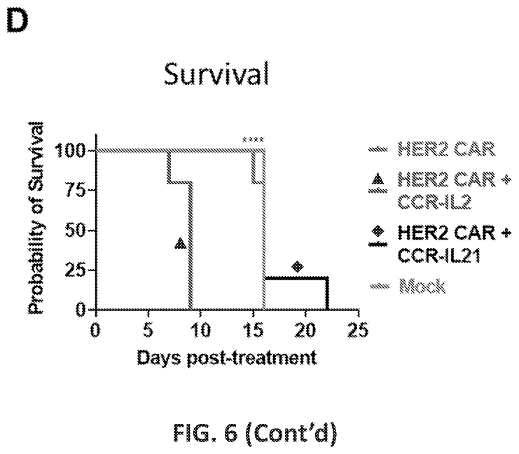

FIG. 6A is a series of bioluminescent images showing the expansion of primary human T cells transduced with a HER2 chimeric antigen receptor (CAR) and a IL-2 or IL-21 CCR in NSG mice bearing 143B osteosarcoma tumors. As can be seen in the data, the IL-2 CCR is a strong driver of T cell proliferation in vivo. Shown in FIG. 6B is quantification of T cell expansion from the bioluminescent images shown in FIG. 6A. As can be seen in the data, the IL-2 CCR is a strong driver of T cell proliferation in vivo. FIG. 6C is a graph showing tumor size of mice that were implanted with 143B osteosarcoma cells and treated with the indicated T cells. FIG. 6D is a graph of survival curves of mice treated with the indicated T cells. Mice that were treated with CCR-IL-2 quickly died from treatment associated toxicity due to uncontrolled proliferation of T cells. Thus, the constitutive IL-2 CCR is a strong driver of proliferation in vivo but results in lethal toxicity in NSG mice.

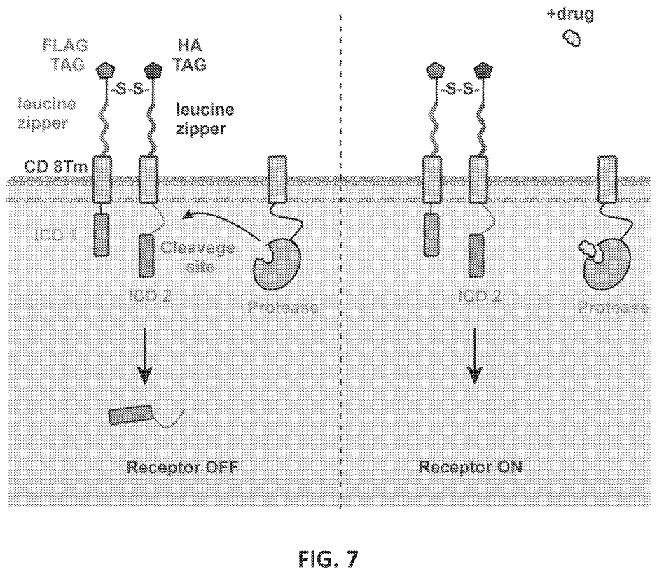

FIG. 7 is a schematic of the drug-regulated chimeric cytokine receptor (CCR) system developed in this example. A protease cleavage site is integrated between the transmembrane domain and intracellular signaling domain of the CCR. Co-expression of a protease that is specific for the cleavage site results in cleavage of the CCR due to proteolytic activity of the protease at the cleavage site, resulting in inactivation of the CCR (receptor OFF). Addition of a protease inhibitor (drug) inhibits this cleavage event and renders the CCR in the on state (Receptor ON). Thus, signaling of the CCR becomes dependent upon the presence of the drug. The regulated CCR could circumvent toxicities associated with constitutive CCRs by regulating proliferation of cells to fall within a therapeutic window.

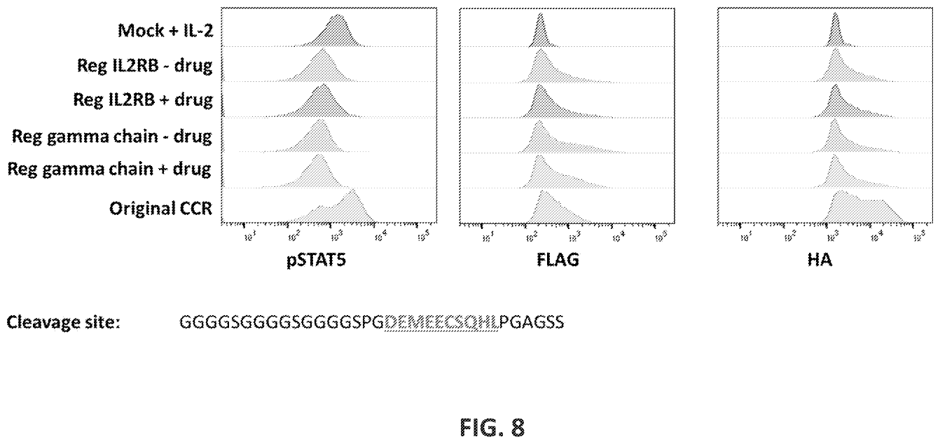

Shown in FIG. 8 is a series of flow plots of primary human T cells transduced with regulated versions of the IL-2R CCR, as described in FIG. 7, demonstrating cell surface expression of HA/FLAG tag and phosphorylated STAT5 (pSTAT5), which is a cytokine signaling molecule downstream of IL-2R signaling. The regulated CCR was constructed by integration of a 33 amino acid linker containing an HCV NS3 protease cleavage between the transmembrane and intracellular domain of the CCR. HCV NS3 protease was co-expressed within the cells to allow for cleavage of the receptor in the absence of protease inhibitor (3 μM grazoprevir, drug). As can be seen in the data, inclusion of the 33 amino acid linker containing a HCV NS3 cleavage site abolishes CCR signaling in the presence or absence of drug and is not an optimal configuration for regulated CCR signaling. Mock non-transduced T cells exposed to exogenously added IL-2 serve as a positive control for pSTAT5.

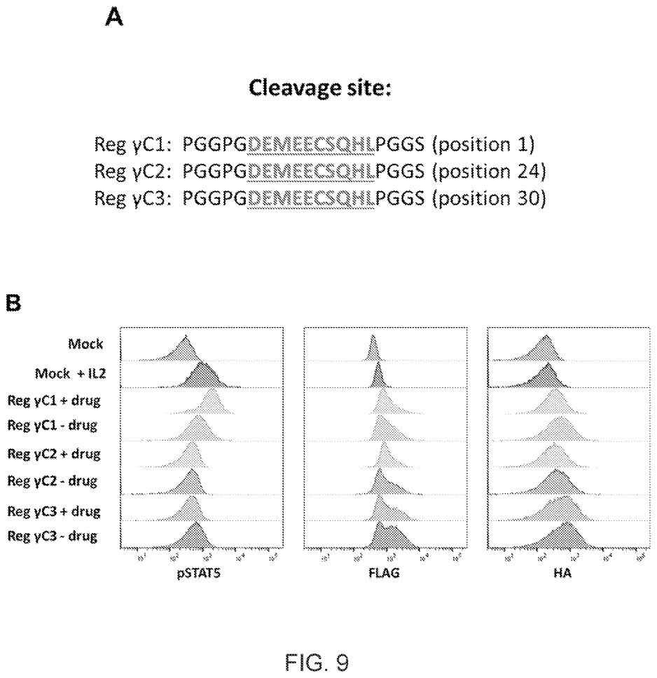

Demonstrated in FIG. 9 is that the insertion of a linker containing a cleavage site upstream of the gamma chain ICD allows for drug regulated CCR signaling. FIG. 9A is a list showing an amino acid linker containing a HCV NS3 protease cleavage site inserted at various positions within the gamma chain ICD. FIG. 9B is a series of flow plots of primary human T cells transduced with regulated versions of the IL-2R CCR, as shown in FIG. 9A, demonstrating cell surface expression of HA/FLAG tag and phosphorylated STAT5 (pSTAT5). The regulated CCR was constructed by integration of a 19 amino acid linker containing an HCV NS3 protease cleavage between the transmembrane and intracellular domain of the gamma chain CCR component. HCV NS3 protease was co-expressed within the cells to allow for cleavage of the receptor in the absence of protease inhibitor (3 μM grazoprevir, drug). As can be seen in the data, inclusion of the 19 amino acid linker containing an HCV NS3 cleavage site at position 1 allows for drug dependent signaling of the CCR. Mock non-transduced T cells exposed to exogenously added IL-2 serve as a positive control for pSTAT5.

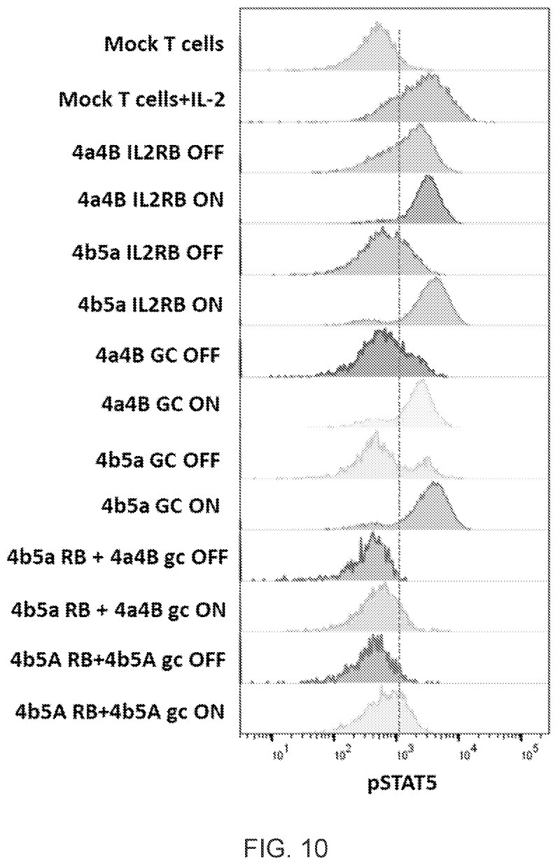

Shown in FIG. 10 is a series of flow plots of primary human T cells transduced with regulated versions of the IL-2R CCR, demonstrating phosphorylated STAT5 (pSTAT5). The regulated CCR was constructed by integration of a 19 amino acid linker containing an HCV NS3 protease cleavage between the transmembrane and intracellular domain of the gamma chain CCR component, the IL-2RB CCR component, or both. HCV NS3 protease was co-expressed within the cells to allow for cleavage of the receptor in the absence of protease inhibitor (drug). A HCV 4a4B or 4b5A cleavage site was used as the HCV cleavage site. “RB” indicates the CCR component containing an IL-2RB ICD. “gc” indicates the CCR component containing a common gamma chain ICD. “OFF” and “ON” indicate in the absence or presence of protease inhibitor (3 μM grazoprevir), respectively. As can be seen in the data, several configurations allow for drug regulated control of CCR signaling. Mock non-transduced T cells exposed to exogenously added IL-2 serve as a positive control for pSTAT5.

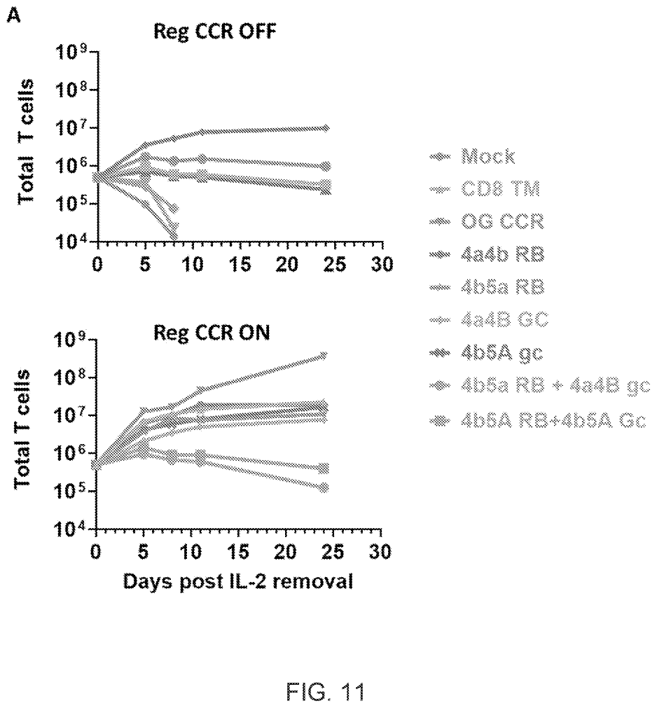

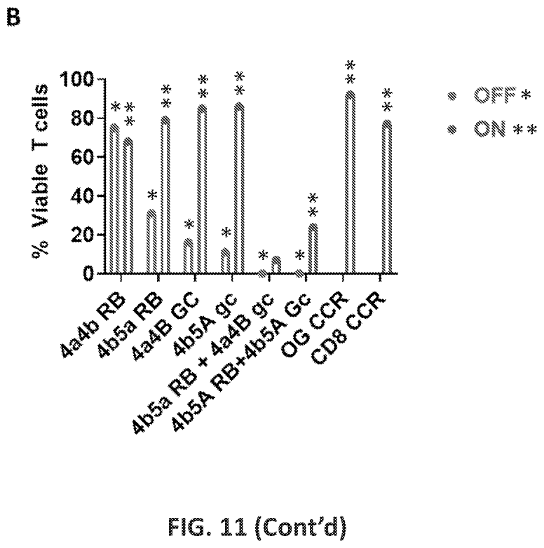

Demonstrated in FIG. 11 is drug control of cell expansion in vitro. FIG. 11A is a series of plots showing in vitro proliferation of primary human T cells transduced with the various CCRs shown in FIG. 10 and grown in the presence (Reg CCR ON) or absence (Reg CCR OFF) of protease inhibitor (3 μM grazoprevir). Original (OG) constitutive IL-2 CCR and non-transduced T cells (Mock) serve as positive and negative controls, respectively. Shown in FIG. 11B are plots showing viability percent of primary human T cells transduced with various CCRs and grown in the presence (ON) or absence (OFF) of protease inhibitor (3 μM grazoprevir). As can be seen in the data, several configurations allow for drug regulated control T cell viability and proliferation.

### Example 3—CCRs Enhance In Vivo Antitumor Activity

Demonstrated in this example is that CCRs enhance in vivo antitumor activity, including following rechallenge.

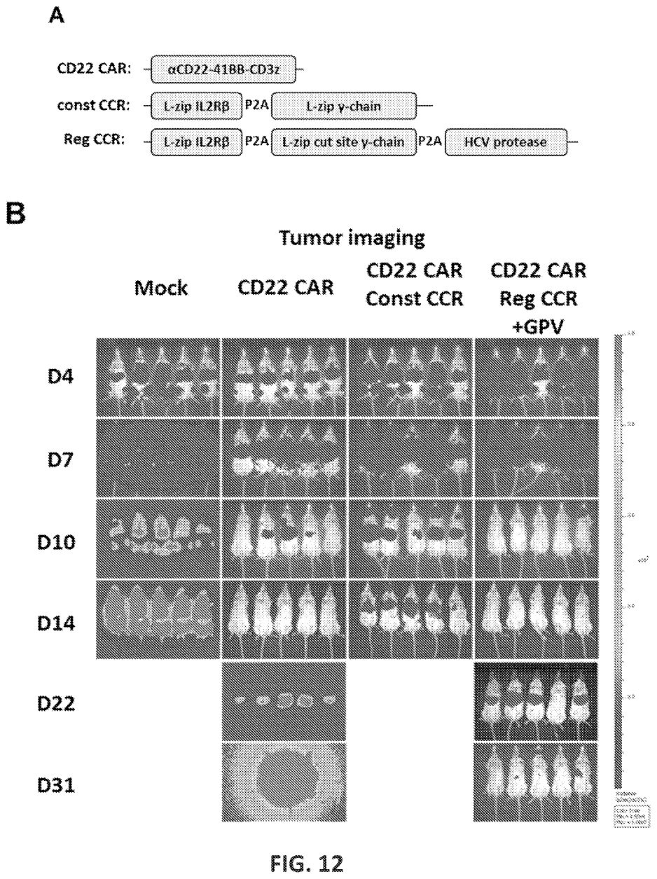

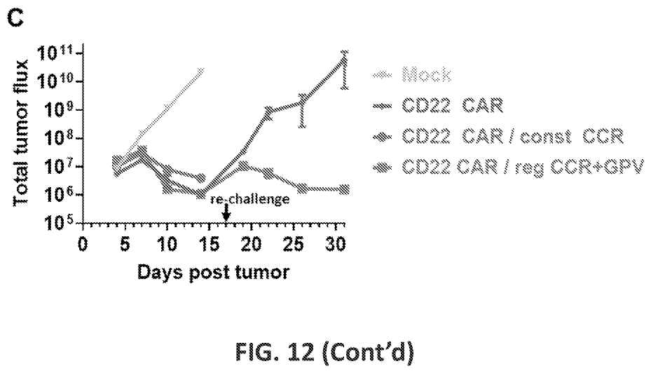

FIG. 12A is a schematic showing the constructs used in this experiment. A CD22 targeting CAR (CD22 CAR) was constructed by fusing an anti-CD22 scFv to a CD8a hinge, CD8a transmembrane domain, 41BB intracellular domain and CD3z signaling domain. A constitutive IL-2 chimeric cytokine receptor (Const CCR) was constructed using the intracellular domains of IL-2RB and common gamma chain and expressed in a bicistronic vector. A drug regulated CCR (Reg CCR) was constructed by integrating a 4b5a HCV cleavage site between the transmembrane domain and common gamma chain intracellular domain. HCV NS3 protease was co-expressed in a tricistronic vector.

Shown in FIG. 12B is a series of bioluminescence images tracking the growth of Nalm6 leukemia, which express firefly luciferase. At day 0, NSG mice were infused with 1×10\hat{ }6 Nalm6, and then treated with the indicated CAR-T cells. Four days later, 4×10\hat{ }6 CAR-T or control cells were implanted by tail vein injection. At Day 17, mice were rechallenged with 4.6×10\hat{ }6 Nalm6 tumor cells. Mice in the “+GPV” group were also implanted with an osmotic drug pump (Azlet model 2002) containing 54 mg/ml grazoprevir and 0.6 mg/mL ritonavir. These mice were additionally dosed with 50 mg/kg grazoprevir and 25 mg/kg ritonavir by oral gavage 1-2 times per day. Mock non-transduced T cells serve as a negative control.

FIG. 12C is a graph showing the quantification of the bioluminescence images shown in FIG. 12B. As can be seen in the data the mice that received CD22 CAR-T cells co-transduced with the regulated IL-2 CCR (Reg CCR) and administered grazoprevir were able to maintain long term tumor control, whereas control CD22 CAR-T cells lacking the regulated CCR succumbed to tumor outgrowth. Mice that received CD22 CAR-T cells co-expressing the constitutive IL-2 CCR (Const CCR) died due to treatment-related toxicity.

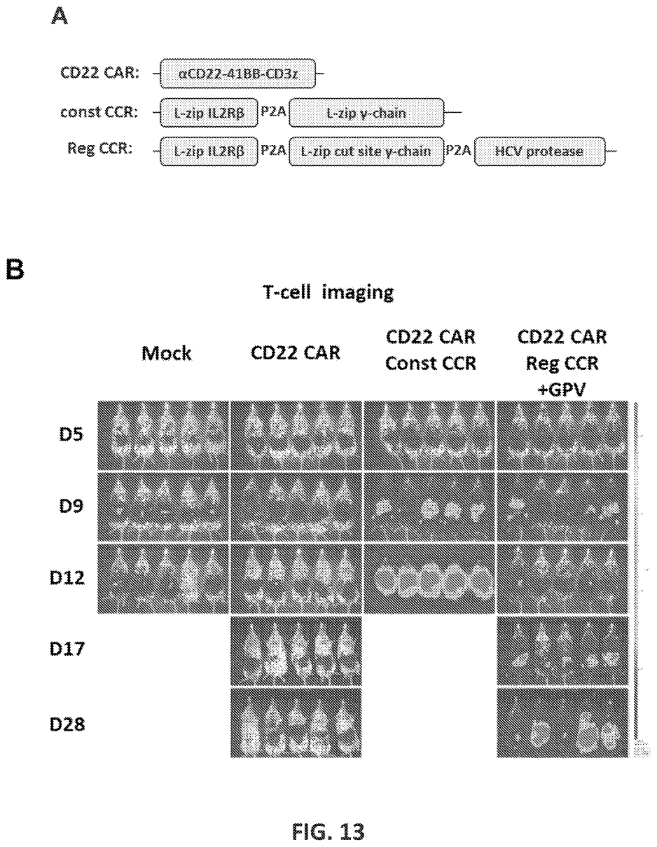

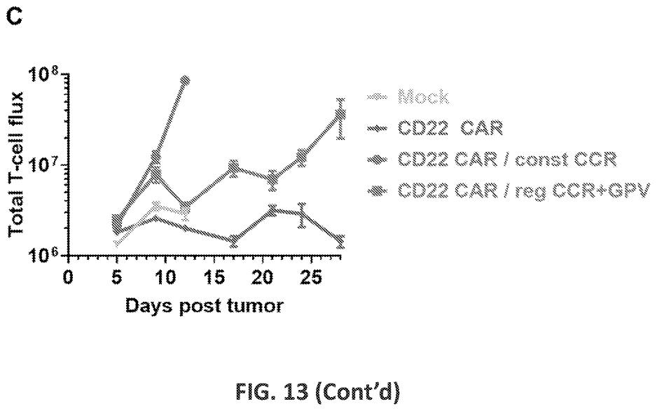

Demonstrated in FIG. 13 is that regulated CCRs expand CAR-T cells in vivo without toxicity. Shown in FIG. 13A is a schematic showing the constructs used in this experiment. A CD22 targeting CAR (CD22 CAR) was constructed by fusing an anti-CD22 scFv to a CD8a hinge, CD8a transmembrane domain, 41BB intracellular domain and CD3z signaling domain. A constitutive IL-2 chimeric cytokine receptor (Const CCR) was constructed using the intracellular domains of IL-2RB and common gamma chain and expressed in a bicistronic vector. A drug regulated CCR (Reg CCR) was constructed by integrating a 4b5a HCV cleavage site between the transmembrane domain and common gamma chain intracellular domain. HCV NS3 protease was co-expressed in a tricistronic vector.

FIG. 13B is a series of bioluminescence images tracking the growth of T cells, which express Antares luciferase, within the mice shown on FIG. 12B. At day 0, NSG mice were infused with 1×10\hat{ }6 Nalm6, and then treated with the indicated CAR-T cells. Four days later, 4×10\hat{ }6 CAR-T or control cells were implanted by tail vein injection. At Day 17, mice were rechallenged with 4.6×10\hat{ }6 Nalm6 tumor cells. Mice in the “+GPV” group were also implanted with an osmotic drug pump (Azlet model 2002) containing 54 mg/ml grazoprevir and 0.6 mg/mL ritonavir. These mice were additionally dosed with 50 mg/kg grazoprevir and 25 mg/kg ritonavir by oral gavage 1-2 times per day. Mock non-transduced T cells serve as a negative control.

Shown in FIG. 13C is a graph showing the quantification of the bioluminescence images shown in FIG. 13B. As can be seen in the data the mice that received CD22 CAR-T cells co-transduced with the regulated IL-2 CCR (Reg CCR) and administered grazoprevir expanded and persisted to a greater degree than control CD22 CAR-T cells lacking the regulated CCR. Mice that received CD22 CAR-T cells co-expressing the constitutive IL-2 CCR (Const CCR) had rapid uncontrolled expansion of CAR-T cells, which led to fatal treatment-related toxicity.

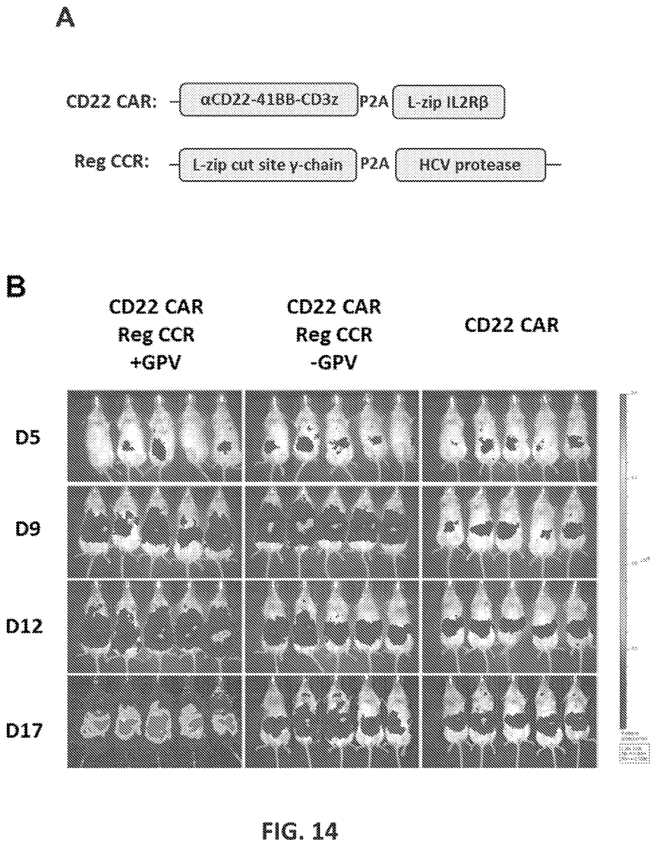

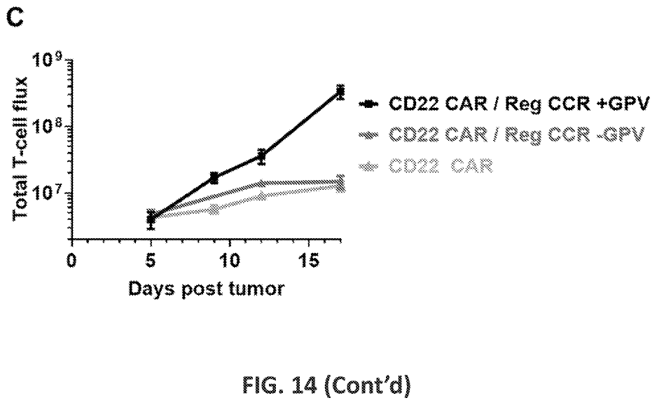

Demonstrated in FIG. 14 is that CAR-T cells expressing the drug regulated IL-2 CCR from two separate vectors have enhanced expansion in vivo in the presence of grazoprevir. FIG. 14A is a schematic showing the constructs used in this experiment. The two chains of the regulated IL-2 CCR were expressed on separate vectors, with the IL-2RB component bicistronically co-expressed with the CD22 CAR. The common gamma chain component, containing a 4b5A HCV cleavage site was co-expressed with HCV NS3 protease on a separate vector.

Shown in FIG. 14B is a series of bioluminescence images tracking the growth of T cells, which express Antares luciferase. At day 0, NSG mice were infused with 1×10\hat{ }6 Nalm6, and then treated with the indicated CAR-T cells. Four days later, 4×10\hat{ }6 CAR-T were implanted by tail vein injection. At Day 17, mice were rechallenged with 4.6×10\hat{ }6 Nalm6 tumor cells. Mice in the “+GPV” group were also implanted with an osmotic drug pump (Azlet model 2002) containing 54 mg/mL grazoprevir and 0.6 mg/mL ritonavir. These mice were additionally dosed with 50 mg/kg grazoprevir and 25 mg/kg ritonavir by oral gavage 1-2 times per day.

FIG. 14C is a graph showing the quantification of the bioluminescence images shown in FIG. 14B. As can be seen in the data the mice that received CD22 CAR-T cells co-expressing the drug regulated IL-2 CCR (Reg CCR) and administered grazoprevir (+GPV) expand to a higher degree in vivo compared to mice that did not receive GPV.

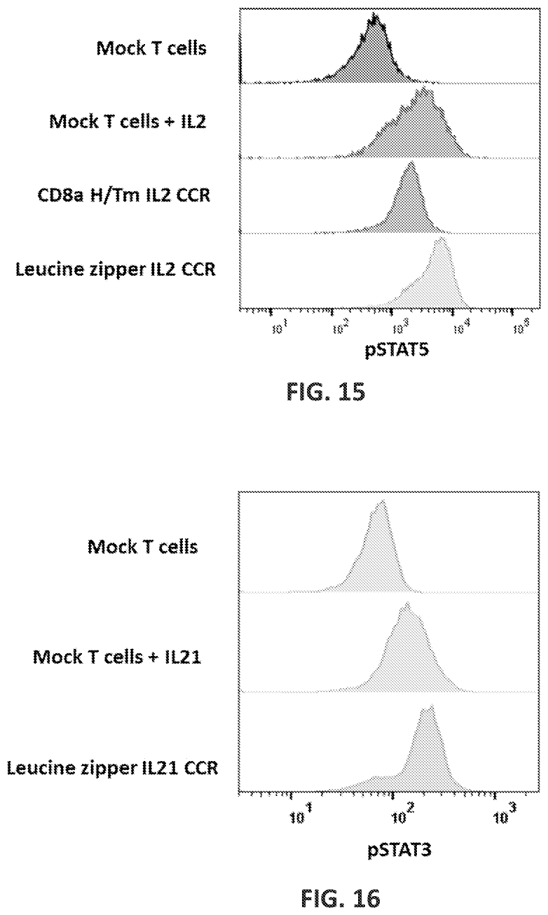

Demonstrated in FIG. 15 is that leucine zipper domains enhance STAT5 signaling in an IL-2 CCR. Shown is a flow plot showing phosphorylation levels of STAT5 from primary human T cells transduced with an IL-2 CCR with (Leucine zipper IL-2 CCR) or without (CD8a H/Tm IL-2 CCR) the leucine zipper components. Mock un-transduced T cells exposed to exogenously added IL-2 serve as a positive control for pSTAT5, while those not exposed to IL-2 serve as a negative control. As can be seen in the data, inclusion of the leucine zipper components in the IL-2 CCR lead to higher levels of phosphorylated STAT5.

Expression of an IL-21 CCR in primary human T cells leading to high phosphorylation levels of STAT3 is demonstrated in FIG. 16. Shown is a flow plot for an IL-21 CCR demonstrating phosphorylation of STAT3 (pSTAT3), which is a cytokine signaling molecule downstream of IL-21R signaling. Mock un-transduced T cells exposed to exogenously added IL-21 serve as a positive control for pSTAT3, while those not exposed to IL-21 serve as a negative control. As can be seen in the data, expression of the IL-21 CCR in primary human T cells leads to high phosphorylation levels of STAT3.

### Example 4—IL-7R and IL-9R CCRs

Described in this example is the development of additional types of CCRs, in particular CCRs based on IL-7R and IL-9R signaling domains (ICDs).

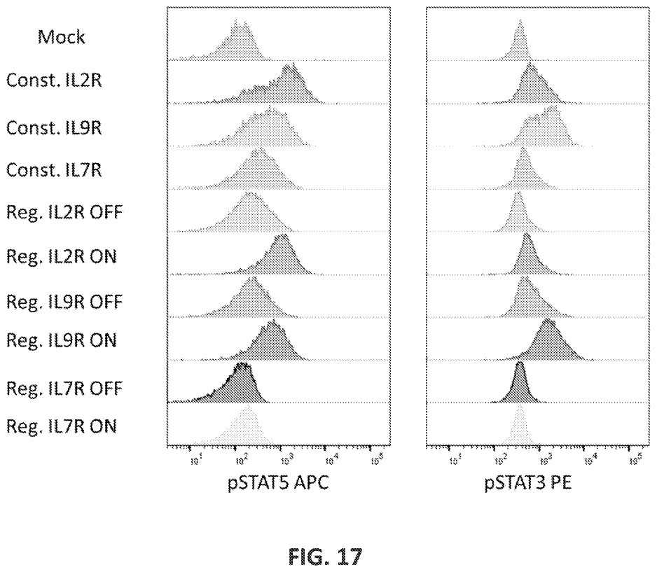

Shown in FIG. 17 is a flow plot for constitutive (Const. IL-2R, Const. IL-9R, Const. IL-7R) or HCV NS3 protease regulated (Reg. IL-2R, Reg. IL-9R, Reg. IL-7R) chimeric cytokine receptors expressed on CD22.BBz CAR T cells. “ON” and “OFF” indicate cell culture in the presence or absence of 3 μM grazoprevir (HCV NS3 protease inhibitor). The HCV NS3 protease regulated receptors contained a HCV 4b5a cleavage site between the transmembrane domain and the gamma chain ICD. These data demonstrate constitutive signaling of IL-2R, IL-9R, and IL-7R CCRs, as determined by high levels of pSTAT5 and/or pSTAT3. In contrast, the HCV NS3 protease regulated chimeric receptors Reg. IL-2R and Reg. IL-9R only express high levels of pSTAT5 and/or pSTAT3 when exposed to the HCV NS3 protease inhibitor grazoprevir. The Reg. IL-7R failed to induce signaling of pSTAT5 and/or pSTAT3 even in the presence of grazoprevir, which suggests that this specific HCV NS3 protease regulated chimeric receptor embodiment was not compatible with IL-7R ICD. The phosphoflow analysis was performed 48 hours after IL-2 was removed from the culture media.

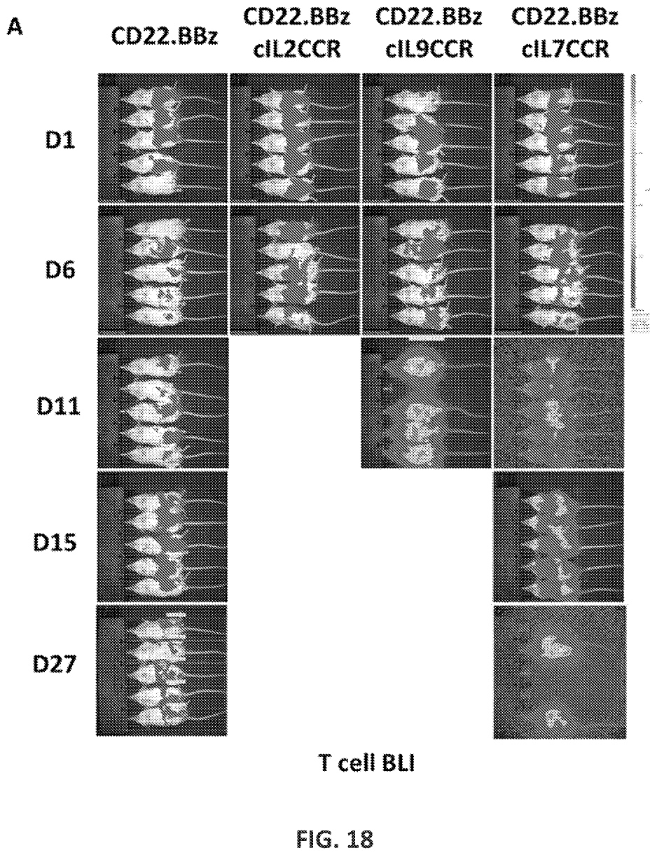

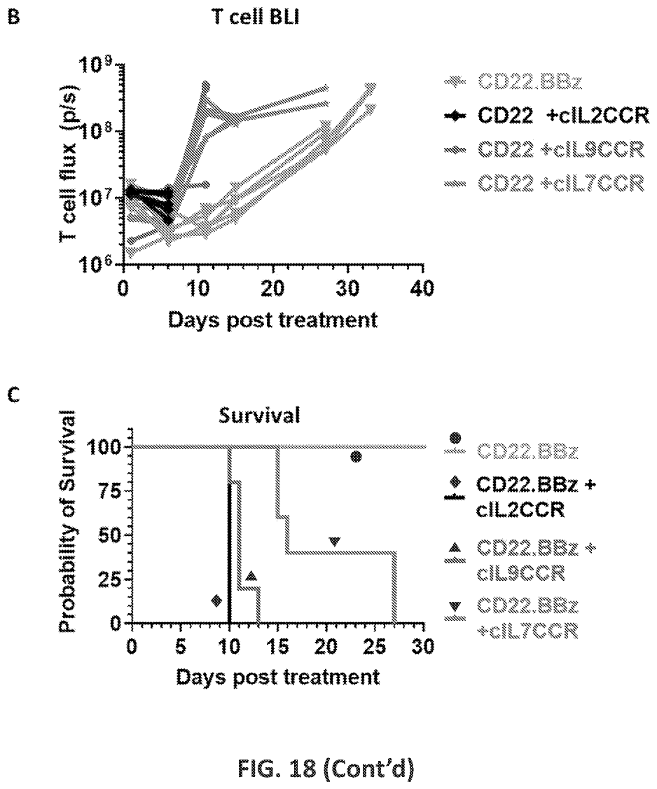

FIG. 18 provides data demonstrating that constitutive IL-2, IL-9, and IL-7 CCRs induce lethal toxicity in mice due to the unregulated outgrowth of CAR-T cells. FIG. 18A is a series of bioluminescence images tracking the growth of T cells, which express Antares luciferase. At day 0, NSG mice were infused with 1×10\hat{ }6 Nalm6. Four days later, 3×10\hat{ }6 of the indicated CAR+CCR T cells were implanted by tail vein injection. At Day 11, mice were rechallenged with 5×10\hat{ }6 Nalm6 tumor cells. FIG. 18B is a plot quantifying the bioluminescence of the images in FIG. 18A. FIG. 18C is a survival curve for the mice shown in FIG. 17A. As can be seen in the data, mice that received constitutive IL2CCR, IL-9CCR, and IL-7CCR experienced lethal toxicity due to the unregulated outgrowth of CAR-T cells.

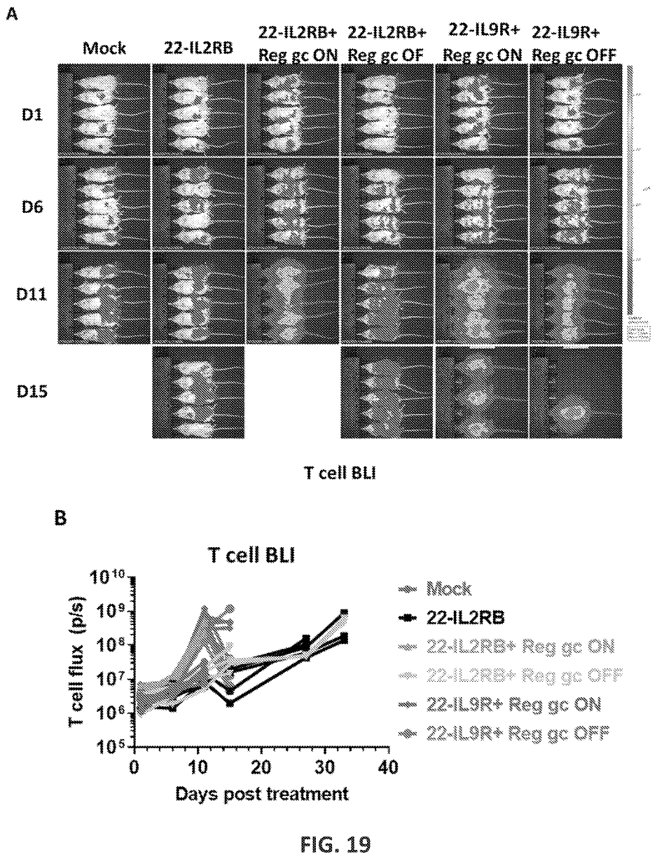

Shown in FIG. 19 is data demonstrating controlled growth of CAR-T cells expressing protease regulated CCRs. FIG. 19A is a series of bioluminescence images tracking the growth of T cells, which express Antares luciferase. At day 0, NSG mice were infused with 1×10\hat{ }6 Nalm6. Four days later, 3×10\hat{ }6 of the indicated CAR+CCR T cells were implanted by tail vein injection. At Day 11, mice were rechallenged with 5×10\hat{ }6 Nalm6 tumor cells. Mice in the “ON” group were also implanted with an osmotic drug pump (Azlet model 2002) containing 54 mg/mL grazoprevir and 0.6 mg/mL ritonavir. These mice were additionally dosed with 50 mg/kg grazoprevir and 25 mg/kg ritonavir by oral gavage once per day. One component of the regulated CCR was delivered on the CD22.BBz CAR vector (i.e., 22-IL-2RB or 22-IL-9R), while the second vector delivered the regulatable gamma chain (Reg gc) component and HCV NS3 protease as shown in FIG. 13A. As can be seen in the data, GPV can drive the growth of T cells expressing regulatable CCRs.

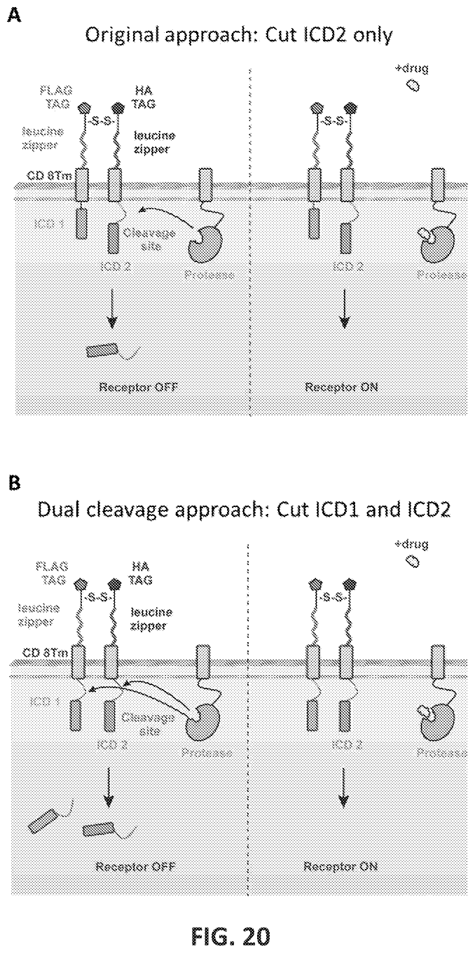

The regulatable IL-9 CCR showed significant outgrowth even in the absence of GPV, which is likely due to the signaling capacity of IL-9R ICD even without a cognate gamma chain ICD. Further iterations of the regulated IL-9 CCR may employ dual cleavage of the IL-9 CCR ICD in addition to cleavage of the gamma chain ICD to reduce this “leaky” activity, as schematically illustrated in FIG. 20.

### Example 5—Regulatable CCRs with a Protease Fused to One of the CCR Subunits

Described in this example is the development of a regulatable CCR comprising a protease fused to one of the CCR subunits.

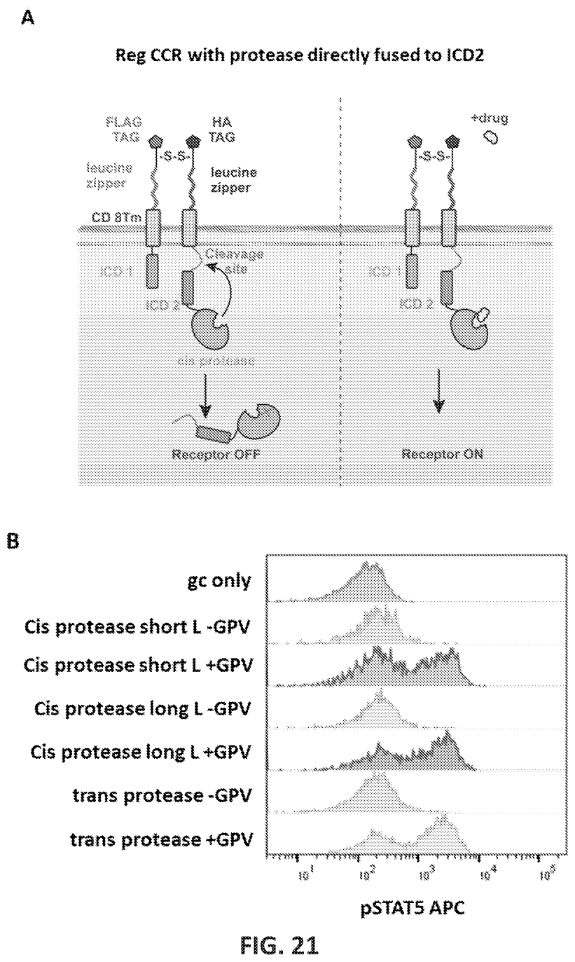

FIG. 21A is a schematic of the Reg CCR with protease directly fused to ICD2 system. A protease cleavage site is integrated between the transmembrane domain and intracellular signaling domain 2 (ICD2). A protease is integrated between ICD2 and the c-terminus of one of the chains/subunits of the CCR. Expression of the protease as part of the subunit results in cleavage of the CCR due to proteolytic activity of the protease at the cleavage site, resulting in inactivation of the CCR (receptor OFF). Addition of a protease inhibitor (drug) inhibits this cleavage event and renders the CCR in the on state (Receptor ON). Thus, signaling of the CCR becomes dependent upon the presence of the drug. The regulatable CCR is expected to circumvent toxicities associated with constitutive CCRs by regulating proliferation of cells to fall within a therapeutic window.

Shown in FIG. 21B is a flow plot showing staining of primary human T cells expressing HCV NS3 protease regulated IL2CCR T cells. “+GPV” and “-GPV” indicate cell culture in the presence or absence of 3 μM grazoprevir (HCV NS3 protease inhibitor), respectively. The HCV NS3 protease regulated receptors contained an HCV 4b5a cleavage site between the transmembrane domain and the gamma chain ICD. The HCV NS3 protease was delivered as a direct fusion to gamma chain ICD with a short linker (Cis protease short L), as a direct fusion to gamma chain ICD with a long linker (Cis protease long L), or in the original approach with the HCV NS3 protease delivered on a separate transmembrane protein (as shown in FIG. 7). These data demonstrate that the HCV NS3 protease regulated chimeric receptors can be regulated with HCV protease expressed in cis as a direct fusion to one of the chains or in trans on a separate transmembrane protein, with high levels of pSTAT5 induced only when exposed to the HCV NS3 protease inhibitor grazoprevir (GPV). The phosphoflow analysis was performed 48 hours after IL-2 was removed from the culture media.

### Example 6—T Cells Co-Expressing a CD22 CAR and a CCR Exhibit Similar Levels of CAR Expression as Compared to T Cells Expressing the CD22 CAR Alone

Demonstrated in this example is that T cells co-expressing a CD22 CAR and a CCR exhibit similar levels of CAR expression as compared to T cells expressing the CD22 CAR alone.

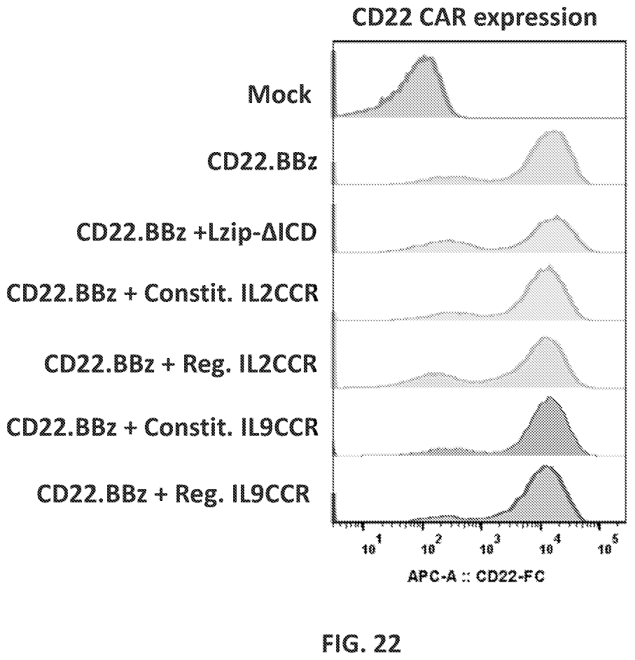

Data is provided in FIG. 22. Shown is a flow plot showing CD22.BBz CAR surface expression levels from primary human T cells transduced with various CCRs. The data demonstrate that all configurations result in similar levels of surface CAR expression. “Constit.” and “Reg.” refer to constitutive and regulatable CCRs, respectively.

### Example 7—CCRs Enhance Proliferation and Anti-Tumor Efficacy of CAR T Cells

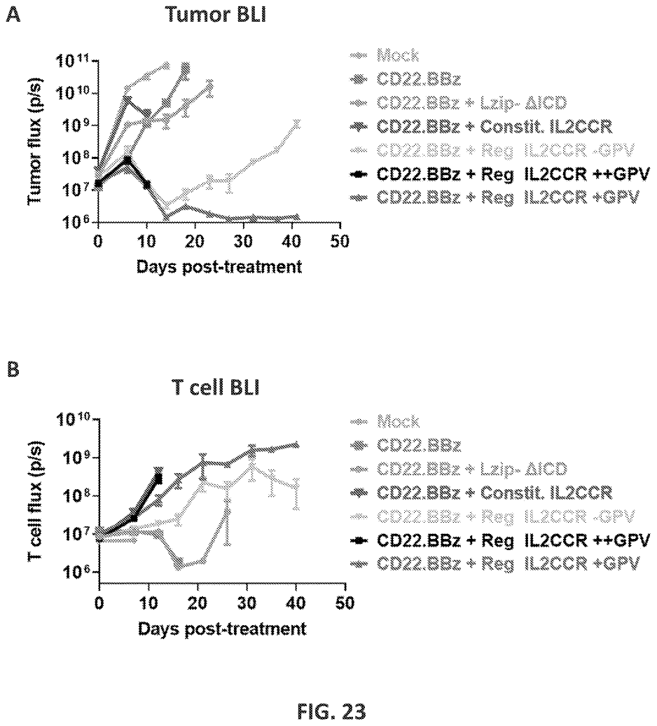

NSG mice were inoculated with Nalm6 leukemia then treated with CD22.BBz CAR-T cells engineered with various IL2 CCR constructs (FIG. 23). GPV was administered to mice daily (++), every 48 hours (+), or not at all (−). Mock untransduced T cells, conventional CD22.BBz CAR-T cells, and CD22.BBz CAR-T cells engineered with a CCR that lacks ICDs (ΔICD) serve as controls. A: Quantification of tumor progression B: Quantification of T cell expansion.

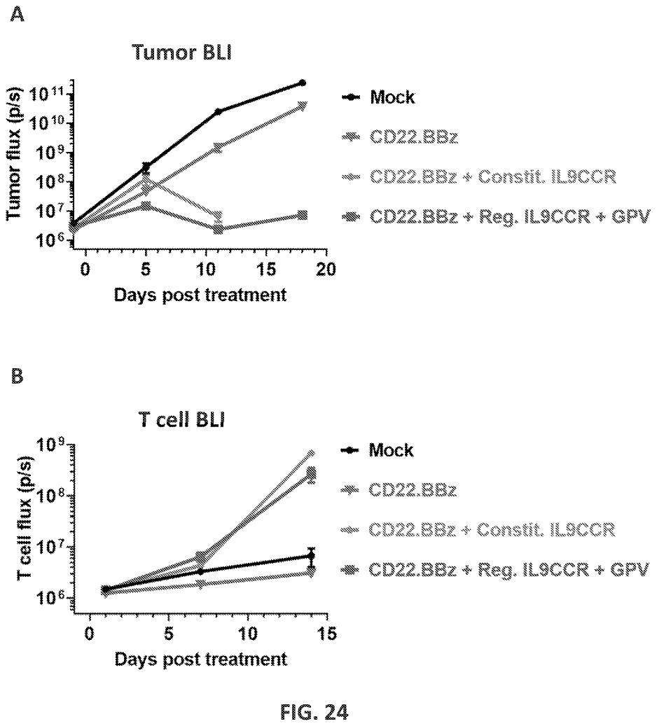

In a further experiment, NSG mice were inoculated with Nalm6 leukemia then treated with CD22.BBz CAR-T cells engineered with various IL9 CCR constructs (FIG. 24). Mock untransduced T cells and conventional CD22.BBz CAR-T cells serve as controls. A: Quantification of tumor progression B: Quantification of T cell expansion.

The data demonstrate that CCRs enhance proliferation and anti-tumor efficacy of CAR T cells.

### Example 8—Drug Control of Cell Expansion of CD22.BBz CAR-T Cells In Vitro

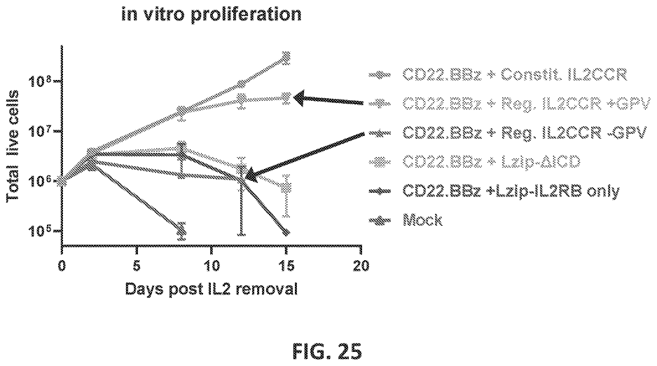

Assessed in this example was drug control of cell expansion of CAR-T cells. CD22.BBz CAR-T cells were cultured in media lacking IL2. As shown in FIG. 25, CD22 CAR-T cells with IL2 CCRs controlled by GPV have enhanced proliferation in vitro, and a remarkable >40 fold expansion of Reg IL2 CCR CAR-T cells in the presence of GPV was observed compared to cells grown in the absence of GPV.

### Example 9—Regulatable CCR-Expressing HER2 CAR-T Cells Express High Levels of pSTAT5 when Cultured in Media Lacking Exogenous Cytokine

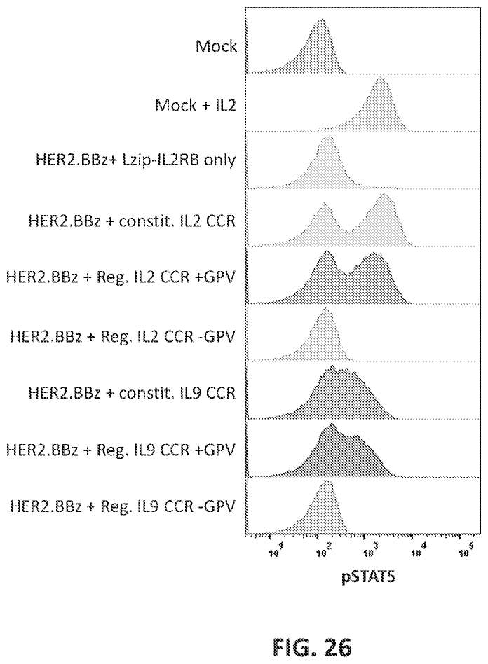

Demonstrated in this example is drug control of pSTAT5 levels of HER2.BBz CAR-T cells in vitro. HER2.BBz CAR-T cells were cultured in media lacking IL2. As shown in FIG. 26, HER2 CAR-T cells engineered with various constitutive and regulatable CCRs express high levels of pSTAT5 when cultured in media lacking exogenous cytokine.

### Example 11—CAR T Cells Expressing a Membrane-Associated IL2RB ICD in the Absence of a Common Gamma Chain ICD Exhibit Enhanced Anti-Tumor Efficacy as Compared to CAR T Cells Lacking the Membrane-Associated IL2RB ICD

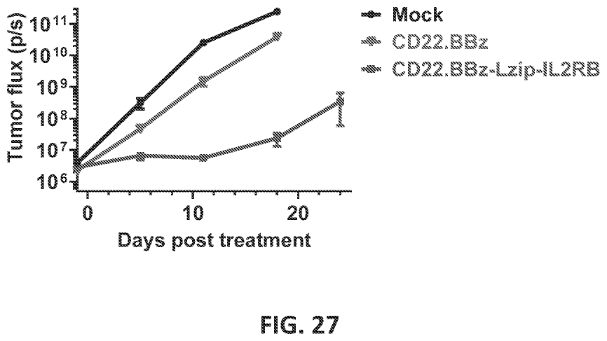

Assessed in this example was anti-tumor efficacy of CAR T cells expressing a membrane-associated IL2RB ICD in the absence of a common gamma chain ICD. NSG mice were inoculated with Nalm6 leukemia then treated with CD22.BBz CAR-T cells engineered with only the IL2RB component of the IL2 CCR. Mock untransduced T cells and conventional CD22.BBz CAR-T cells serve as controls. The data show quantification of tumor progression (FIG. 27). The data unexpectedly demonstrate that CAR T cells expressing a membrane-associated IL2RB ICD in the absence of a common gamma chain ICD exhibit enhanced anti-tumor efficacy as compared to CAR T cells lacking the membrane-associated IL2RB ICD.

### Example 12—In Vitro Expression and In Vivo Activity of CAR-T Cells Engineered with Non-Gamma Chain CCRs

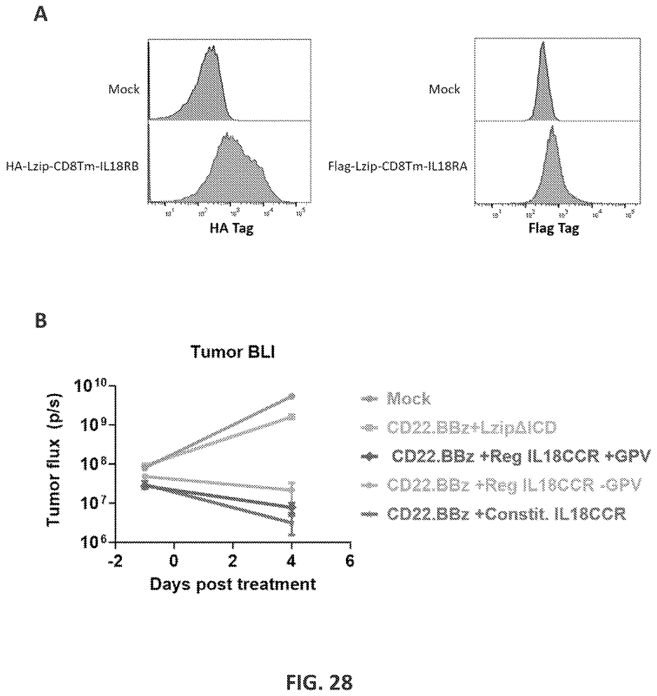

Assessed in this example was in vitro expression and in vivo activity of CAR-T cells engineered with non-gamma chain CCRs (IL18 CCRs as proof of concept in this example). Shown in FIG. 28A are flow plots demonstrating cell surface expression of CCRs with IL18RA and IL18RB ICDs As shown in FIG. 28B, NSG mice were inoculated with Nalm6 leukemia then treated with CD22.BBz CAR-T cells engineered with regulated or constitutive IL18CCR constructs. For regulated IL18CCR groups, GPV was administered to mice daily (+GPV), or not at all (−GPV). Mock untransduced T cells and CD22.BBz CAR-T cells engineered with a CCR that lacks ICDs (ΔICD) serve as controls. The data show quantification of tumor progression.

### Methods

### Construction of Retroviral Plasmid Vectors

DNA sequences were synthesized as oligonucleotides or gBlocks (Integrated DNA Technologies) In-Fusion cloning (Takara Bio) was used to clone DNA sequences into the MSGV1 retroviral vector. Products of the In-Fusion reaction were transformed into chemically competent cells (Stellar Cell, Takara Bio) using the heat shock method. The resulting colonies were sequence verified using Sanger sequencing. Bacteria cultures were grown for 16 hours at in LB ampicillin media. Subsequently, plasmid DNA was extracted from bacteria cultures using a miniprep kit (QIAprep Spin Miniprep Kit, Qiagen).

Isolation of Primary Human T Cells from Blood Donors

Primary human T cells were extracted by negative selection from buffy coats using the RosetteSep Human T cell Enrichment kit (Stem Cell Technologies) and SepMate-50 tubes. T cells were cryopreserved in cryopreservation media (CryoStor CS10, Stem Cell Technologies) until use.

### Retrovirus Production

293GP packaging cells and RD114 envelope plasmid were used to prepare retroviral supernatant RD114. Briefly, 11 μg of RD114 and 22 μg of the corresponding MSGV1 transfer plasmid were mixed in Lipofectamine 2000 reagent (Thermo Fisher) and used to transfect 293GP cells, which were grown to about 80% confluency on poly-D-lysine cell culture dishes (Corning). 293GP cells were cultured in 293GP culture media (DMEM, 10% FBS, 2 mM L-glutamine, 10 mM HEPES, and 100 μg/mL streptomycin, 100 U/mL penicillin, Gibco) in a 5% CO₂ environment at 37° C. This media was replenished every 24 hours. After 48 and 72-hour post transfection, retroviral supernatant was harvested, centrifuged to get rid of dead cells and debris, then stored at −80 C until further use.

### T Cell Retroviral Transductions

At Day 0, primary human T cells were thawed and activated using anti-CD3/CD28 Human T-Expander Dynabeads (Thermo Fisher) at a 3:1 bead to cell ratio. Two days later, retroviral supernatant was spun onto 12-well or 24 well cell culture plates that had been pre-coated with RetroNectin (Takara Bio) according to the manufacturer's instructions. Approximately 0.1-1 mL of retroviral supernatant diluted in DMEM was added to retronectin plates and centrifuged at 3200 RPM, 32° C. for about two hours. The supernatant was then aspirated off of the wells. Subsequently, T cells prepared at a concentration of 0.25-0.5×10⁶ cells per mL were added in 1 mL of T cell media comprised of: AIM V (Thermo Fisher), 5% fetal bovine serum (FBS), 2 mM L-glutamine (Gibco), 100 mg/mL streptomycin (Gibco), 100 U/mL penicillin (Gibco), 10 mM HEPES (Gibco), and 100 U/mL rhIL-2 (Peprotech)). The plates were then gently spun down at 1200 RPM for 2 min then incubated overnight at 37° C. 5% CO₂. This transduction process was repeated the next day. At Day 4, Dynabeads were removed by magnetic separation. Cells were cultured at a concentration of 0.4-2×10⁶ cells/mL and expanded for up to several weeks.

### In Vitro Cell Proliferation Assay

To determine if CCR-transduced T cells could proliferate in the absence of exogenously added cytokine, cells were harvested from culture, centrifuged, and resuspended in T cell culture media lacking T cell growth cytokines (i.e. IL-7, IL-2, IL15, etc). Cell numbers and viability were determined using trypan blue exclusion test for viability and a Cellometer Auto 1000 cell counter (Nexcelom Biosciences).

### Antibodies and Flow Cytometry

The following antibodies were used for staining cells: Brilliant Violet 421™ anti-DYKDDDDK Tag Antibody (Biolegend), anti-HA.11 Epitope Tag Antibody (BioLegend), Alexa Fluor® 647 Mouse Anti-Stat5 (pY694) (BD), and PE Mouse Anti-Stat3 (pY705) Clone 4/P-STAT3 (BD). Recombinant CD22-Fc, fluorescently labeled with the DyLight 650 Microscale Antibody Labeling Kit (Thermo Fisher), was used for CAR detection. Cells were stained according to the antibody manufacturer's instructions. A BD Fortessa instrument was used to run flow cytometry samples. Data were analyzed using FlowJo software (Tree Star). Intracellular phospho-flow cytometry was performed to assess phosphorylation levels of STAT3 and STAT5 using Life Technologies Fix & perm kit with methanol modification (Thermo Fisher Scientific).

### Grazoprevir Preparation and Administration

For in vitro experiments, DMSO was used to reconstitute grazoprevir (Acme Bioscience, custom synthesis) as a 1000× stock solution (3 mM) and frozen at −80° C. For in vivo mouse experiments, 100% PEG 300 (Rigaku) was used to reconstitute grazoprevir potassium salt (Acme Bioscience, custom synthesis) to 60 mg/ml by incubation in a 37° C. water bath and vortexing. Ritonavir (ACROS Organic), used as a pharmacokinetic enhancer was similarly reconstituted to 60 mg/mL using a 1:1 solution of ethanol and propylene glycol (MP Biomedicals). Oral formulations were prepared at 50 mg/kg grazoprevir and 25 mg/kg ritonavir in gavage diluent (70% (v/v) PEG 300, 5% (w/v) sucrose (Sigma), and 5% (v/v) DPBS (Gibco)), and delivered to mice by oral gavage using 20 gauge 30 mm feeding tubes (Instech) in a total volume of 100 μL per dose.

### Nalm6 Leukemia Mouse Model

Six to ten-week old male or female NSG mice were implanted with 1×10⁶ Nalm6 leukemia cells which were transduced with firefly luciferase to monitor tumor progression. Four days later, 4×10\hat{ }6 CAR-T or control cells were implanted by tail vein injection. The T cells were transduced with Antares luciferase to allow for in vivo monitoring. At Day 17, mice were rechallenged with 4.6×10\hat{ }6 Nalm6 tumor cells

### 143B Osteosarcoma Mouse Model

Six to ten-week old male or female NSG mice were implanted with 1×10⁶ 143B cells, prepared in 100 μL DPBS, into the tibia periosteum. Four days later, mice were treated with 3×10\hat{ }6 human T cells expressing the indicated transgenes by tail vein injection. Caliper measurements were used to monitor tumor progression. The T cells were transduced with Antares luciferase to allow for in vivo monitoring.

### In Vivo Bioluminescence Imaging

Tumor progression was monitored using the firefly luciferase assay. Mice were administered 200 μL of 15 μg/mL D-luciferin to detect tumor cells expressing firefly luciferase. T cell expansion was monitored using the Antares luciferase assay. Mice were administered 200 μL of a 1:40 dilution of Nano-Glo substrate (Promega, diluted in DPBS) by intraperitoneal injection to detect T cells expressing Antares luciferase. An IVIS imaging system was used to acquire images 5 min after injection using 30 sec exposures and medium binning. If pixels were saturated in the image, an additional image was acquired using the auto-expose setting. Living Image software was used to calculate total flux around a region of interest around the body of each mouse. Non-saturated images were used to quantify BLI images. BLI images within the same experiment were set to the same scale.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. One or more nucleic acids encoding a first subunit of a chimeric cytokine receptor and a second subunit of the chimeric cytokine receptor, wherein:
the first subunit comprises a first heterologous dimerization domain and a first cytokine receptor intracellular signaling domain (ICD); and
the second subunit comprises a second heterologous dimerization domain cognate for the first heterologous dimerization domain, and a second cytokine receptor ICD.

2. The one or more nucleic acids of claim 1, wherein the first and second heterologous dimerization domains are extracellular dimerization domains.

3. The one or more nucleic acids of claim 1 or claim 2, wherein the first and second heterologous dimerization domains each comprise a leucine zipper domain, a BTB (BR-C, ttk and bab) domain, a POZ (Pox virus and Zinc finger) domain, a coiled coil domain, or a PDZ domain.

4. The one or more nucleic acids of claim 1 or claim 2, wherein the first and second heterologous dimerization domains comprise, respectively, a constant heavy chain and constant light chain of an IgG, IgE, or IgD antibody, or dimerizing fragments thereof.

5. The one or more nucleic acids of claim 1, wherein:
the first subunit comprises a heterologous transmembrane domain which is the first heterologous dimerization domain; and
the second subunit comprises a heterologous transmembrane domain which is the second heterologous dimerization domain.

6. The one or more nucleic acids of claim 5, wherein the heterologous transmembrane domains of the first and second subunits are independently selected from the group consisting of: a CD8α transmembrane domain, a CD28 transmembrane domain, a HER2 transmembrane domain, and an EGFR transmembrane domain.

7. The one or more nucleic acids of any one of claims 1 to 4, wherein the first and second subunits comprise a transmembrane domain independently selected from a HER2, EGFR, IL-2RB, IL-7R, IL-21R, IL-4R, IL-9R, IL15Rα, common gamma chain (γc), Eph receptor, VEGF receptor, ErbB receptor, FGF receptor, ROR1, ROR2 PDGF receptor, MET receptor, CD35, CD37, CD3γ, CD30, CD4, CD5, CD9, CD16, CD22, CD27, CD28, CD33, CD37, CD45, CD64, CD80, CD86, CD134, CD137, CD152, CD154, and PD-1 transmembrane domain.

8. The one or more nucleic acids of any one of claims 1 to 7, wherein the first cytokine receptor ICD, the second cytokine receptor ICD, or both, comprises an IL-2RB ICD, IL-3Rα ICD, IL-4R ICD, IL-5Rα ICD, IL-6Rα ICD, IL-7R ICD, IL-9R ICD, IL-10Rα ICD, IL-10Rβ ICD, IL-11Rα ICD, IL-12Rβ1 ICD, IL-12Rβ2 ICD, IL-13Rα1 ICD, IL-13Rα2 ICD, IL-20Rα ICD, IL-20Rβ, IL-21R ICD, IL-22Rα1, IL-22Rα2, IL-23R ICD, IL-27Rα ICD, IL-28RA ICD, IL-31Rα ICD, TGFβR1 ICD, TGFβR2 ICD, TGFβR3 ICD, IFNAR1 ICD, IFNAR2 ICD, IFNGR1 ICD, IFNGR2 ICD, IFNLR ICD, CSF2RB ICD, gp130 ICD, CD9 ICD, OSMR ICD, CSF-1R ICD, or any combination thereof.

9. The one or more nucleic acids of any one of claims 1 to 7, wherein the first cytokine receptor ICD comprises an ICD from a common cytokine receptor gamma-chain family.

10. The one or more nucleic acids of claim 9, wherein the ICD from a common cytokine receptor gamma-chain family is an IL-2RB ICD, IL-4R ICD, IL-7R ICD, IL-9R ICD, or an IL-21R ICD.

11. The one or more nucleic acids of any one of claims claim 9 or claim 10, wherein the second cytokine receptor ICD is a common gamma chain (γc) ICD.

12. The one or more nucleic acids of claim 11, wherein the second subunit comprises a transition domain disposed between the transmembrane domain and the γc ICD.

13. The one or more nucleic acids of claim 12, wherein the transition domain comprises a juxtamembrane (JM) region of a cell surface receptor.

14. The one or more nucleic acids of claim 13, wherein the transition domain comprises a JM region of CD8a or IL-15Rα.

15. The one or more nucleic acids of any one of claims 1 to 14, wherein the first subunit and the second subunit are encoded by a single nucleic acid.

16. The one or more nucleic acids of claim 15, wherein the single nucleic acid provides for cistronic expression of the first and second subunits.

17. The one or more nucleic acids of claim 16, wherein the single nucleic acid comprises a ribosome skipping element disposed between the region encoding the first subunit and the region encoding the second subunit.

18. The one or more nucleic acids of any one of claims 1 to 14, wherein the first subunit is encoded by a first nucleic acid and the second subunit is encoded by a second nucleic acid.

19. The one or more nucleic acids of any one of claims 1 to 18, wherein the first subunit, the second subunit, or both, comprises a protease cleavage site disposed between the transmembrane domain and the ICD.

20. The one or more nucleic acids of claim 19, wherein the protease cleavage site is a viral protease cleavage site.

21. The one or more nucleic acids of claim 20, wherein the viral protease cleavage site is a hepatitis C virus (HCV) nonstructural protein 3 (NS3) cleavage site.

22. The one or more nucleic acids of claim 21, wherein the viral protease cleavage site is selected from the group consisting of: an NS4A/4B junction cleavage site, an NS3/NS4A junction cleavage site, an NS4A/NS4B junction cleavage site, an NS4B/NS5A junction cleavage site, an NS5A/NS5B junction cleavage site, and variants thereof cleavable by the viral protease.

23. The one or more nucleic acids of claim 20, wherein the viral protease cleavage site is an HIV protease cleavage site.

24. The one or more nucleic acids of claim 20, wherein the viral protease cleavage site is a SARS-COV-2 protease cleavage site.

25. The one or more nucleic acids of claim 19, wherein the protease cleavage site is a human protease cleavage site.

26. The one or more nucleic acids of claim 25, wherein the human protease cleavage site is a cleavage site for human renin protease, human kallikrein (KLK) protease, human enterokinase protease, human thrombin, a human matrix metalloprotease (MMP), human urokinase-type plasminogen activator receptor (uPAR), human plasmin, or human cathepsin.

27. The one or more nucleic acids of any one of claims 19 to 26, wherein the one or more nucleic acids further encode a protease, wherein the protease cleavage site is a cleavage site for the protease.

28. The one or more nucleic acids of claim 27, wherein the one or more nucleic acids encode a fusion protein comprising the protease and a transmembrane domain.

29. The one or more nucleic acids of claim 27, wherein the protease is a soluble cytosolic protease.

30. The one or more nucleic acids of claim 27, wherein the one or more nucleic acids encode:
the protease fused to the first subunit;
the protease fused to the second subunit; or
the protease fused to the first subunit and the protease fused to the second subunit.

31. The one or more nucleic acids of any one of claims 27 to 30, wherein proteolytic activity of the protease is regulatable.

32. The one or more nucleic acids of claim 31, wherein proteolytic activity of the protease is regulatable via a cell-permeable small molecule.

33. The one or more nucleic acids of claim 31 or claim 32, wherein the cell-permeable small molecule is an inhibitor of the protease.

34. The one or more nucleic acids of claim 33, wherein the protease is derived from HCV NS3, and wherein the inhibitor of the protease is selected from the group consisting of: asunaprevir (ASV), danoprevir (DPV), simeprevir (SPV), grazoprevir (GPV), glecaprevir, voxilaprevir, and any combination thereof.

35. The one or more nucleic acids of claim 33, wherein the protease is an HIV protease, and wherein the inhibitor of the protease is selected from the group consisting of: atazanavir (Reyataz), darunavir (Prezista), fosamprenavir (Lexiva), indinavir (Crixivan), lopinavir/ritonavir (Kaletra), nelfinavir (Viracept), ritonavir (Norvir), saquinavir (Invirase), tipranavir (Aptivus), atazanavir/cobicistat (Evotaz), darunavir/cobicistat (Prezcobix), and any combination thereof.

36. The one or more nucleic acids of claim 33, wherein the protease is a SARS-COV-2 protease, and wherein the inhibitor of the protease is nirmatrelvir (Paxlovid).

37. The one or more nucleic acids of claim 33, wherein the protease is a human renin protease, and wherein the inhibitor of the protease is aliskiren.

38. The one or more nucleic acids of any one of claims 1 to 37, wherein the first subunit, the second subunit, or both, comprises an extracellular protein tag.

39. The one or more nucleic acids of claim 38, wherein the extracellular protein tag is an N-terminal protein tag.

40. The one or more nucleic acids of claim 38 or claim 39, wherein the extracellular protein tag is a FLAG tag or an HA tag.

41. The one or more nucleic acids of any one of claims 1 to 40, wherein the first subunit and the second subunit each comprise an extracellular cysteine residue, wherein disulfide bonding between the extracellular cysteine residue of the first subunit and the extracellular cysteine residue of the second subunit stabilizes the association of the first subunit and the second subunit when expressed on the surface of a cell.

42. A chimeric cytokine receptor comprising the first and second subunits encoded by the one or more nucleic acids of any one of claims 1 to 41.

43. One or more expression constructs comprising the one or more nucleic acids of any one of claims 1 to 41 operably linked to one or more promoters.

44. A nucleic acid encoding a membrane associated polypeptide comprising a transmembrane domain and a cytokine receptor ICD, wherein the polypeptide does not comprise an extracellular cytokine binding domain.

45. The nucleic acid of claim 44, wherein the cytokine receptor ICD is an IL-2RB ICD, an IL-7R ICD, an IL-9R ICD, or a common gamma chain (γc) ICD.

46. The nucleic acid of claim 45, wherein the cytokine receptor ICD is an IL-2RB ICD.

47. The nucleic acid of any one of claims 44 to 46, wherein the polypeptide comprises a heterologous dimerization domain.

48. The nucleic acid of claim 47, wherein the heterologous dimerization domain is an extracellular heterologous dimerization domain.

49. The nucleic acid of claim 47 or 48, wherein the heterologous dimerization domain is as defined in claim 3 or 4.

50. The nucleic acid of any one of claims 44 to 49, wherein the polypeptide comprises a protease cleavage site disposed between the transmembrane domain and the ICD.

51. The nucleic acid of claim 50, wherein the protease cleavage site is as defined in any one of claims 20 to 26.

52. A polypeptide encoded by the nucleic acid of any one of claims 44 to 51.

53. An expression construct comprising the nucleic acid of any one of claims 44 to 51 operably linked to a promoter.

54. A cell comprising the one or more nucleic acids of any one of claims 1 to 41.

55. A cell comprising the nucleic acid of any one of claims 44 to 51.

56. A cell comprising the one or more expression constructs of claim 43.

57. A cell comprising the expression construct of claim 53.

58. The cell of claim 57, wherein the cell does not express a cytokine receptor subunit comprising a common gamma chain (γc) ICD.

59. The cell of any one of claims 54 to 58, wherein the cell is a human cell.

60. The cell of any one of claims 54 to 59, wherein the cell is an immune cell.

61. The cell of claim 60, wherein the immune cell is a T cell.

62. The cell of any one of claims 54 to 61, wherein the one or more nucleic acids further encode a chimeric antigen receptor (CAR), an engineered T cell receptor (TCR), a synthetic Notch (SynNotch) receptor, a Modular Extracellular Sensor Architecture (MESA) receptor, a Tango receptor, a ChaCha receptor, a generalized extracellular molecule sensor (GEMS) receptor, a cytokine receptor, a chemokine receptor, a switch receptor, an adhesion molecule, an integrin, an inhibitory receptor, a stimulatory receptor, an immunoreceptor tyrosine-based activation motif (ITAM)-containing receptor, or an immunoreceptor tyrosine-based inhibition motif (ITIM)-containing receptor.

63. The cell of any one of claims 54 to 61, wherein the one or more nucleic acids further encode a CAR.

64. A composition comprising a population of cells as defined in any one of claims 56 to 63, wherein the composition is suitable for administration to a subject in need thereof.

65. A method of administering a cell-based therapy to a subject in need thereof, the method comprising administering an effective amount of the composition of claim 64 to the subject, wherein the cells express: the chimeric cytokine receptor comprising the first and second subunits encoded by the one or more nucleic acids; or the membrane associated polypeptide encoded by the nucleic acid.

66. The method according to claim 65, wherein signaling of the chimeric cytokine receptor or membrane associated polypeptide is regulatable.

67. The method according to claim 66, wherein the cells express the chimeric cytokine receptor comprising the first and second subunits encoded by the one or more nucleic acids, and wherein:
the first subunit, the second subunit, or both, comprises a protease cleavage site disposed between the transmembrane domain and the ICD;
the one or more nucleic acids further encode a protease, wherein the protease cleavage site is a cleavage site for the protease; and
proteolytic activity of the protease is regulatable.

68. The method according to claim 66, wherein the cells express the membrane associated polypeptide, and wherein the membrane associated polypeptide comprises a protease cleavage site disposed between the transmembrane domain and the cytokine receptor ICD.

69. The method according to claim 67 or 68, wherein proteolytic activity of the protease is regulatable via a cell-permeable small molecule.

70. The method according to claim 69, wherein the cell-permeable small molecule is an inhibitor of the protease, and wherein the method further comprises administering an effective amount of the cell-permeable small molecule to the subject when signaling by the chimeric cytokine receptor or membrane associated polypeptide is desired.

71. The method according to claim 70, comprising ceasing administration of the cell-permeable small molecule when signaling by the chimeric cytokine receptor or membrane associated polypeptide is no longer desired.

72. The method according to claim 70 or claim 71, wherein the protease is derived from HCV NS3.

73. The method according to claim 72, wherein the inhibitor of the protease is selected from the group consisting of: asunaprevir (ASV), danoprevir (DPV), simeprevir (SPV), grazoprevir (GPV), glecaprevir, voxilaprevir, and any combination thereof.

74. The method according to claim 70 or claim 71, wherein the protease is an HIV protease, and wherein the inhibitor of the protease is selected from the group consisting of: atazanavir (Reyataz), darunavir (Prezista), fosamprenavir (Lexiva), indinavir (Crixivan), lopinavir/ritonavir (Kaletra), nelfinavir (Viracept), ritonavir (Norvir), saquinavir (Invirase), tipranavir (Aptivus), atazanavir/cobicistat (Evotaz), darunavir/cobicistat (Prezcobix), and any combination thereof.

75. The method according to claim 70 or claim 71, wherein the protease is a SARS-COV-2 protease, and wherein the inhibitor of the protease is nirmatrelvir (Paxlovid).

76. The method according to claim 70 or claim 71, wherein the protease is a human renin protease, and wherein the inhibitor of the protease is aliskiren.

77. The method according to any one of claims 65 to 76, wherein the subject in need thereof has cancer, and wherein the cell-based therapy is administered to treat the cancer.

78. The method according to claim 77, wherein the cancer comprises a solid tumor.

79. The method according to claim 78, wherein the solid tumor is a carcinoma, lymphoma, blastoma, or sarcoma.

80. The method according to claim 77, wherein the cancer comprises a hematological malignancy.

81. The method according to claim 80, wherein the hematological malignancy is a leukemia, a lymphoma, or multiple myeloma.


SEQUENCE LISTING (63 sequences)

SEQ ID NO: 1 - protein, 435 aa, synthetic construct
MLLLVTSLLLCELPHPAFLLIPDYKDDDDKCGGKGGGLEIRAAFLRRRNTALRTRVAELRQRVQRARNRVSQYRTRYGPLTTTPAPRPPTPAPTIASQPLSLRPEACRPAAGGAVHTRGLDFACDIYIWAPLAGTCGVLLLSLVITLYCNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 2 - protein, 188 aa, synthetic construct
MLLLVTSLLLCELPHPAFLLIPDYKDDDDKCGGKGGGLEIRAAFLRRRNTALRTRVAELRQRVQRARNRVSQYRTRYGPLTTTPAPRPPTPAPTIASQPLSLRPEACRPAAGGAVHTRGLDFACDIYIWAPLAGTCGVLLLSLVITLYCKSRQTPPLASVEMEAMEALPVTWGTSSRDEDLENCSHHL

SEQ ID NO: 3 - protein, 434 aa, synthetic construct
MLLLVTSLLLCELPHPAFLLIPDYKDDDDKCGGKGGGLEIRAAFLRRRNTALRTRVAELRQRVQRARNRVSQYRTRYGPLTTTPAPRPPTPAPTIASQPLSLRPEACRPAAGGAVHTRGLDFACDIYIWAPLAGTCGVLLLSLVITLYCSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 4 - protein, 239 aa, synthetic construct
MLLLVTSLLLCELPHPAFLLIPYPYDVPDYACGGSSGLEIEAAFLEQENTALETEVAELEQEVQRLENEVSQYETRYGPLGGGKTTTPAPRPPTPAPTIASQPLSLRPEACRPAAGGAVHTRGLDFACDIYIWAPLAGTCGVLLLSLVITLYCERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 5 - protein, 244 aa, synthetic construct
MLLLVTSLLLCELPHPAFLLIPYPYDVPDYACGGSSGLEIEAAFLEQENTALETEVAELEQEVQRLENEVSQYETRYGPLGGGKTTTPAPRPPTPAPTIASQPLSLRPEACRPAAGGAVHTRGLDFACDIYIWAPLAGTCGVLLLSLVITLYCGGSGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 6 - protein, 255 aa, synthetic construct
MLLLVTSLLLCELPHPAFLLIPYPYDVPDYACGGSSGLEIEAAFLEQENTALETEVAELEQEVQRLENEVSQYETRYGPLGGGKTTTPAPRPPTPAPTIASQPLSLRPEACRPAAGGAVHTRGLDFACDIYIWAPLAGTCGVLLLSLVITLYCNHRNRRRVCKCPRPVVERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 7 - protein, 246 aa, synthetic construct
MLLLVTSLLLCELPHPAFLLIPYPYDVPDYACGGSSGLEIEAAFLEQENTALETEVAELEQEVQRLENEVSQYETRYGPLGGGKTTTPAPRPPTPAPTIASQPLSLRPEACRPAAGGAVHTRGLDFACDIYIWAPLAGTCGVLLLSLVITLYCKSRQTPPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 8 - protein, 706 aa, synthetic construct
MLLLVTSLLLCELPHPAFLLIPDYKDDDDKCGGKGGGLEIRAAFLRRRNTALRTRVAELRQRVQRARNRVSQYRTRYGPLTTTPAPRPPTPAPTIASQPLSLRPEACRPAAGGAVHTRGLDFACDIYIWAPLAGTCGVLLLSLVITLYCNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVSIGATNFSLLKQAGDVEENPGPGSTMLLLVTSLLLCELPHPAFLLIPYPYDVPDYACGGSSGLEIEAAFLEQENTALETEVAELEQEVQRLENEVSQYETRYGPLGGGKTTTPAPRPPTPAPTIASQPLSLRPEACRPAAGGAVHTRGLDFACDIYIWAPLAGTCGVLLLSLVITLYCKSRQTPPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 9 - protein, 725 aa, synthetic construct
MLLLVTSLLLCELPHPAFLLIPDYKDDDDKCGGKGGGLEIRAAFLRRRNTALRTRVAELRQRVQRARNRVSQYRTRYGPLTTTPAPRPPTPAPTIASQPLSLRPEACRPAAGGAVHTRGLDFACDIYIWAPLAGTCGVLLLSLVITLYCNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVSIGATNFSLLKQAGDVEENPGPGSTMLLLVTSLLLCELPHPAFLLIPYPYDVPDYACGGSSGLEIEAAFLEQENTALETEVAELEQEVQRLENEVSQYETRYGPLGGGKTTTPAPRPPTPAPTIASQPLSLRPEACRPAAGGAVHTRGLDFACDIYIWAPLAGTCGVLLLSLVITLYCKSRQTPPPGGPGDEMEECSQHLPGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 10 - protein, 726 aa, synthetic construct
MLLLVTSLLLCELPHPAFLLIPDYKDDDDKCGGKGGGLEIRAAFLRRRNTALRTRVAELRQRVQRARNRVSQYRTRYGPLTTTPAPRPPTPAPTIASQPLSLRPEACRPAAGGAVHTRGLDFACDIYIWAPLAGTCGVLLLSLVITLYCNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVSIGATNFSLLKQAGDVEENPGPGSTMLLLVTSLLLCELPHPAFLLIPYPYDVPDYACGGSSGLEIEAAFLEQENTALETEVAELEQEVQRLENEVSQYETRYGPLGGGKTTTPAPRPPTPAPTIASQPLSLRPEACRPAAGGAVHTRGLDFACDIYIWAPLAGTCGVLLLSLVITLYCKSRQTPPPGGPGECTTPCSGSWLPGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 11 - protein, 385 aa, synthetic construct
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSSGNSSGGSTGCVVIVGRIVLSGSGTSAPITAYAQQTRGLLGCIITSLTGRDKNQVEGEVQIMSTATQTFLATCINGVCWTVYHGAGTRTIASPKGPVIQMYTNVDQDLVGWPAPQGSRSLTPCTCGSSDLYLVTRHADVIPVRRRGDGRGSLLSPRPISYLKGSSGGPLLCPAGHAVGLFRAAVCTRGVAKAVDFIPVENLETTMRSPVFTDNSSPPAVTLTHNAS

SEQ ID NO: 12 - protein, 865 aa, synthetic construct
MLLLVTSLLLCELPHPAFLLIPDYKDDDDKCGGKGGGLEIRAAFLRRRNTALRTRVAELRQRVQRARNRVSQYRTRYGPLTTTPAPRPPTPAPTIASQPLSLRPEACRPAAGGAVHTRGLDFACDIYIWAPLAGTCGVLLLSLVITLYCKPGGPGECTTPCSGSWLAAANCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVSIGATNFSLLKQAGDVEENPGPGSTMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSSGNSSGGSTGCVVIVGRIVLSGSGTSAPITAYAQQTRGLLGCIITSLTGRDKNQVEGEVQIMSTATQTFLATCINGVCWTVYHGAGTRTIASPKGPVIQMYTNVDQDLVGWPAPQGSRSLTPCTCGSSDLYLVTRHADVIPVRRRGDGRGSLLSPRPISYLKGSSGGPLLCPAGHAVGLFRAAVCTRGVAKAVDFIPVENLETTMRSPVFTDNSSPPAVTLTHNAS

SEQ ID NO: 13 - protein, 676 aa, synthetic construct
MLLLVTSLLLCELPHPAFLLIPYPYDVPDYACGGSSGLEIEAAFLEQENTALETEVAELEQEVQRLENEVSQYETRYGPLGGGKTTTPAPRPPTPAPTIASQPLSLRPEACRPAAGGAVHTRGLDFACDIYIWAPLAGTCGVLLLSLVITLYCKSRQTPPPGGPGECTTPCSGSWLPGGSERTMPRIPTLKNLEDLVTEYHGNFSAWSGVSKGLAESLQPDYSERLCLVSEIPPKGGALGEGPGASPCNQHSPYWAPPCYTLKPETSIGATNFSLLKQAGDVEENPGPGSTMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSSGNSSGGSTGCVVIVGRIVLSGSGTSAPITAYAQQTRGLLGCIITSLTGRDKNQVEGEVQIMSTATQTFLATCINGVCWTVYHGAGTRTIASPKGPVIQMYTNVDQDLVGWPAPQGSRSLTPCTCGSSDLYLVTRHADVIPVRRRGDGRGSLLSPRPISYLKGSSGGPLLCPAGHAVGLFRAAVCTRGVAKAVDFIPVENLETTMRSPVFTDNSSPPAVTLTHNAS

SEQ ID NO: 14 - protein, 1133 aa, synthetic construct
MLLLVTSLLLCELPHPAFLLIPYPYDVPDYACGGSSGLEIEAAFLEQENTALETEVAELEQEVQRLENEVSQYETRYGPLGGGKTTTPAPRPPTPAPTIASQPLSLRPEACRPAAGGAVHTRGLDFACDIYIWAPLAGTCGVLLLSLVITLYCKSRQTPPPGGPGECTTPCSGSWLPGGSERTMPRIPTLKNLEDLVTEYHGNFSAWSGVSKGLAESLQPDYSERLCLVSEIPPKGGALGEGPGASPCNQHSPYWAPPCYTLKPETSIGATNFSLLKQAGDVEENPGPGSTMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSSGNSSGGSTGCVVIVGRIVLSGSGTSAPITAYAQQTRGLLGCIITSLTGRDKNQVEGEVQIMSTATQTFLATCINGVCWTVYHGAGTRTIASPKGPVIQMYTNVDQDLVGWPAPQGSRSLTPCTCGSSDLYLVTRHADVIPVRRRGDGRGSLLSPRPISYLKGSSGGPLLCPAGHAVGLFRAAVCTRGVAKAVDFIPVENLETTMRSPVFTDNSSPPAVTLTHNASATNFSLLKQAGDVEENPGPGSTMLLLVTSLLLCELPHPAFLLIPDYKDDDDKCGGKGGGLEIRAAFLRRRNTALRTRVAELRQRVQRARNRVSQYRTRYGPLTTTPAPRPPTPAPTIASQPLSLRPEACRPAAGGAVHTRGLDFACDIYIWAPLAGTCGVLLLSLVITLYCNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 15 - protein, 196 aa, synthetic construct
MLLLVTSLLLCELPHPAFLLIPYPYDVPDYACGGTTTPAPRPPTPAPTIASQPLSLRPEACRPAAGGAVHTRGLDFACDIYIWAPLAGTCGVLLLSLVITLYCKSRQTPPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 16 - protein, 389 aa, synthetic construct
MLLLVTSLLLCELPHPAFLLIPYPYDVPDYACGGTTTPAPRPPTPAPTIASQPLSLRPEACRPAAGGAVHTRGLDFACDIYIWAPLAGTCGVLLLSLVITLYCNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 17 - protein, 484 aa, synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 944 aa, synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSIGATNFSLLKQAGDVEENPGPGSTMLLLVTSLLLCELPHPAFLLIPDYKDDDDKCGGKGGGLEIRAAFLRRRNTALRTRVAELRQRVQRARNRVSQYRTRYGPLTTTPAPRPPTPAPTIASQPLSLRPEACRPAAGGAVHTRGLDFACDIYIWAPLAGTCGVLLLSLVITLYCNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 19 - protein, 508 aa, synthetic construct
MLLLVTSLLLCELPHPAFLLIPYPYDVPDYACGGSSGLEIEAAFLEQENTALETEVAELEQEVQRLENEVSQYETRYGPLGGGKTTTPAPRPPTPAPTIASQPLSLRPEACRPAAGGAVHTRGLDFACDIYIWAPLAGTCGVLLLSLVITLYCKSRQTPPGGPGECTTPCSGSWLPGGSERTMPRIPTLKNLEDLVTEYHGNFSAWSGVSKGLAESLQPDYSERLCLVSEIPPKGGALGEGPGASPCNQHSPYWAPPCYTLKPETGSTGGGSGGGSGGGSGSSGNSSGGSTGCVVIVGRIVLSGSGTSAPITAYAQQTRGLLGCIITSLTGRDKNQVEGEVQIMSTATQTFLATCINGVCWTVYHGAGTRTIASPKGPVIQMYTNVDQDLVGWPAPQGSRSLTPCTCGSSDLYLVTRHADVIPVRRRGDGRGSLLSPRPISYLKGSSGGPLLCPAGHAVGLFRAAVCTRGVAKAVDFIPVENLETTMRSPVFTDNSSPPAVTLTHNAS

SEQ ID NO: 20 - protein, 650 aa, synthetic construct
MLLLVTSLLLCELPHPAFLLIPDYKDDDDKCGGKGGGLEIRAAFLRRRNTALRTRVAELRQRVQRARNRVSQYRTRYGPLTTTPAPRPPTPAPTIASQPLSLRPEACRPAAGGAVHTRGLDFACDIYIWAPLAGTCGVLLLSLVITLYCKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFSIGATNFSLLKQAGDVEENPGPGSTMLLLVTSLLLCELPHPAFLLIPYPYDVPDYACGGSSGLEIEAAFLEQENTALETEVAELEQEVQRLENEVSQYETRYGPLGGGKTTTPAPRPPTPAPTIASQPLSLRPEACRPAAGGAVHTRGLDFACDIYIWAPLAGTCGVLLLSLVITLYCKSRQTPPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 21 - protein, 615 aa, synthetic construct
MLLLVTSLLLCELPHPAFLLIPDYKDDDDKCGGKGGGLEIRAAFLRRRNTALRTRVAELRQRVQRARNRVSQYRTRYGPLTTTPAPRPPTPAPTIASQPLSLRPEACRPAAGGAVHTRGLDFACDIYIWAPLAGTCGVLLLSLVITLYCKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSIGATNFSLLKQAGDVEENPGPGSTMLLLVTSLLLCELPHPAFLLIPYPYDVPDYACGGSSGLEIEAAFLEQENTALETEVAELEQEVQRLENEVSQYETRYGPLGGGKTTTPAPRPPTPAPTIASQPLSLRPEACRPAAGGAVHTRGLDFACDIYIWAPLAGTCGVLLLSLVITLYCKSRQTPPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 22 - protein, 888 aa, synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSIGATNFSLLKQAGDVEENPGPGSTMLLLVTSLLLCELPHPAFLLIPDYKDDDDKCGGKGGGLEIRAAFLRRRNTALRTRVAELRQRVQRARNRVSQYRTRYGPLTTTPAPRPPTPAPTIASQPLSLRPEACRPAAGGAVHTRGLDFACDIYIWAPLAGTCGVLLLSLVITLYCKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 23 - protein, 853 aa, synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSIGATNFSLLKQAGDVEENPGPGSTMLLLVTSLLLCELPHPAFLLIPDYKDDDDKCGGKGGGLEIRAAFLRRRNTALRTRVAELRQRVQRARNRVSQYRTRYGPLTTTPAPRPPTPAPTIASQPLSLRPEACRPAAGGAVHTRGLDFACDIYIWAPLAGTCGVLLLSLVITLYCKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 24 - protein, 658 aa, synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSIGATNFSLLKQAGDVEENPGPGSTMLLLVTSLLLCELPHPAFLLIPDYKDDDDKCGGKGGGLEIRAAFLRRRNTALRTRVAELRQRVQRARNRVSQYRTRYGPLTTTPAPRPPTPAPTIASQPLSLRPEACRPAAGGAVHTRGLDFACDIYIWAPLAGTCGVLLLSLVITLYC

SEQ ID NO: 25 - protein, 849 aa, synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSIGATNFSLLKQAGDVEENPGPGSTMLLLVTSLLLCELPHPAFLLIPDYKDDDDKCGGKGGGLEIRAAFLRRRNTALRTRVAELRQRVQRARNRVSQYRTRYGPLTTTPAPRPPTPAPTIASQPLSLRPEACRPAAGGAVHTRGLDFACDIYIWAPLAGTCGVLLLSLVITLYCYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 26 - protein, 382 aa, synthetic construct
MLLLVTSLLLCELPHPAFLLIPYPYDVPDYACGGSSGLEIEAAFLEQENTALETEVAELEQEVQRLENEVSQYETRYGPLGGGKTTTPAPRPPTPAPTIASQPLSLRPEACRPAAGGAVHTRGLDFACDIYIWAPLAGTCGVLLLSLVITLYCKSRQTPPSALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW

SEQ ID NO: 27 - protein, 811 aa, synthetic construct
MLLLVTSLLLCELPHPAFLLIPYPYDVPDYACGGSSGLEIEAAFLEQENTALETEVAELEQEVQRLENEVSQYETRYGPLGGGKTTTPAPRPPTPAPTIASQPLSLRPEACRPAAGGAVHTRGLDFACDIYIWAPLAGTCGVLLLSLVITLYCKSRQTPPPGGPGECTTPCSGSWLPGGSALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEWGSTATNFSLLKQAGDVEENPGPGSTMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSSGNSSGGSTGCVVIVGRIVLSGSGTSAPITAYAQQTRGLLGCIITSLTGRDKNQVEGEVQIMSTATQTFLATCINGVCWTVYHGAGTRTIASPKGPVIQMYTNVDQDLVGWPAPQGSRSLTPCTCGSSDLYLVTRHADVIPVRRRGDGRGSLLSPRPISYLKGSSGGPLLCPAGHAVGLFRAAVCTRGVAKAVDFIPVENLETTMRSPVFTDNSSPPAVTLTHNAS

SEQ ID NO: 28 - protein, 10 aa, Hepacivirus hominis
DEMEECSQHL

SEQ ID NO: 29 - protein, 11 aa, Hepacivirus hominis
ECTTPCSGSWL

SEQ ID NO: 30 - protein, 10 aa, Hepacivirus hominis
EDVVPCSMGS

SEQ ID NO: 31 - DNA, 1305 bp, synthetic construct
  source 1..1305: mol_type = other DNA
atgcttctgctcgtgacaagcctgctgctgtgcgagctgccccaccctgcctttctgctgatccctgactacaaagacgacgatgacaagtgcggtggaaaaggcggaggcttggaaattagagcagcatttcttaggcgcagaaacaccgcgctccgaaccagagtagccgagctgagacaacgggtacaaagggcaaggaatcgcgtgagtcaatatcgaactcgatacggccccctcacaaccacaccagctcctagacccccaacgcccgcccccactatagcaagtcagcccctcagccttcgaccagaggcatgccggccagcggcaggaggagcggtacatactagagggttggactttgcgtgcgacatatatatctgggcaccgctggcggggacatgtggcgtactccttctgtcattggtaattactctttactgtaattgtcgcaacacagggccttggttgaaaaaagttctcaaatgcaatacgccggacccatctaaatttttcagtcagctcagctctgagcatgggggagatgtgcaaaaatggctcagctcccccttcccatctagtagcttcagcccgggcggtctcgctccggagatatcccccctggaggtcctggagagggacaaggtaacacaactgctcctccaacaggacaaagtcccggagcccgcgagtttgagctcaaaccactctctgacgtcttgtttcactaatcagggttacttttttttccatctcccggatgctctcgaaattgaggcttgtcaggtatacttcacttacgatccgtattccgaggaagatccggacgagggagtcgcgggtgctcctaccggctcaagtccccagccgctccagccattgagcggagaggacgacgcatattgtacgtttccgtctcgggacgacctcctcctcttctccccttccttgcttggaggtccaagtcctccgtcaacagcacctgggggaagtggtgccggtgaggagcggatgcctccatccttgcaggaacgggtaccgagggactgggaccctcagccacttgggcctccgacgcctggtgtgccagatctcgttgacttccagcccccgccggaattggttctgcgagaggctggtgaagaggtgccagatgctgggcctcgagaaggtgtttcatttccatggagtaggcctccgggacagggagagttccgcgcgctcaatgcacgccttcctctgaatactgatgcctacctgagtcttcaggagctccagggccaagacccaacacatctggtt

SEQ ID NO: 32 - DNA, 564 bp, synthetic construct
  source 1..564: mol_type = other DNA
atgcttctgctcgtgacaagcctgctgctgtgcgagctgccccaccctgcctttctgctgatccctgactacaaagacgacgatgacaagtgcggtggaaaaggcggaggcttggaaattagagcagcatttcttaggcgcagaaacaccgcgctccgaaccagagtagccgagctgagacaacgggtacaaagggcaaggaatcgcgtgagtcaatatcgaactcgatacggccccctcacaaccacaccagctcctagacccccaacgcccgcccccactatagcaagtcagcccctcagccttcgaccagaggcatgccggccagcggcaggaggagcggtacatactagagggttggactttgcgtgcgacatatatatctgggcaccgctggcggggacatgtggcgtactccttctgtcattggtaattactctttactgtaaaagtcgccaaaccccaccgctcgcatccgtcgaaatggaagcaatggaggcgctgccggttacatgggggacatcttctcgcgatgaagatttggaaaattgtagccatcatctc

SEQ ID NO: 33 - DNA, 1302 bp, synthetic construct
  source 1..1302: mol_type = other DNA
atgcttctgctcgtgacaagcctgctgctgtgcgagctgccccaccctgcctttctgctgatccctgactacaaagacgacgatgacaagtgcggtggaaaaggcggaggcttggaaattagagcagcatttcttaggcgcagaaacaccgcgctccgaaccagagtagccgagctgagacaacgggtacaaagggcaaggaatcgcgtgagtcaatatcgaactcgatacggccccctcacaaccacaccagctcctagacccccaacgcccgcccccactatagcaagtcagcccctcagccttcgaccagaggcatgccggccagcggcaggaggagcggtacatactagagggttggactttgcgtgcgacatatatatctgggcaccgctggcggggacatgtggcgtactccttctgtcattggtaattactctttactgttctttgaagacacatccgttgtggcgcctctggaagaaaatatgggcagtgccttctccagagcgatttttcatgccactctataaagggtgctcaggtgactttaaaaagtgggttggcgcgccttttacggggagttctctcgaactcggcccgtggtctccggaagttccatcaaccttggaagtttacagctgccaccctcctcggtctcctgcgaagcgactgcagctgacggaactccaagaacctgctgagcttgtcgaaagcgatggagtacctaaaccctcattctggcccactgcccagaatagtgggggttctgcatattccgaagaaagggatcgcccgtacggccttgttagtatagacactgtgactgttttggatgctgaaggaccatgtacctggccctgttcttgcgaggatgatggttacccagcactcgacctggatgctggtttggaacctagtcccgggctggaggaccccctcttggacgctggaactacggttctctcttgcggctgcgtgtctgctgggagtcccgggctcggtggcccacttggtagtcttctggataggcttaagccacctctggccgacggagaagactgggccggcggattgccttggggaggtagatcccctggaggggtctccgaatctgaggcgggctcacctctggcagggttggatatggacacatttgactctggttttgtgggctccgactgttcctccccggtggaatgtgacttcacgtctccaggtgatgagggtccaccgcgaagttacttgaggcaatgggtagtaatcccacctcccctctccagccctgggcctcaagcctca

SEQ ID NO: 34 - DNA, 717 bp, synthetic construct
  source 1..717: mol_type = other DNA
atgctgttattggtcacatcactgctcctctgtgaactgcctcatcccgcttttttattgattccctatccctacgatgtgcccgattacgcttgcggtggaagtagcgggctcgaaatagaagcagccttcctggaacaggaaaatactgcgctggagaccgaggtggcagaacttgagcaagaagtgcaacggctggagaacgaggtctctcaatacgaaacgcggtatggaccgctgggcggaggaaagactactacaccggcacccaggccacctacacctgcaccgaccattgcttcacaaccactcagtctccgacctgaagcctgtagacctgctgctggcggtgcagtccataccagaggactcgattttgcttgcgacatttatatatgggcacctttggcaggaacctgcggggtattgcttctttccctggtgatcacactgtattgtgaacgaacaatgccgcgcatcccgactctcaagaaccttgaggatcttgtcacggagtatcatggaaacttctccgcatggagcggcgtctcaaaaggcttggccgagtctcttcaaccagattactcagaaagattgtgcctggtctccgaaattccacctaaaggcggagccctcggcgaaggcccaggagcaagcccgtgcaaccagcactccccgtattgggcaccaccatgctacaccctgaagccagaaacc

SEQ ID NO: 35 - DNA, 732 bp, synthetic construct
  source 1..732: mol_type = other DNA
atgctgttattggtcacatcactgctcctctgtgaactgcctcatcccgcttttttattgattccctatccctacgatgtgcccgattacgcttgcggtggaagtagcgggctcgaaatagaagcagccttcctggaacaggaaaatactgcgctggagaccgaggtggcagaacttgagcaagaagtgcaacggctggagaacgaggtctctcaatacgaaacgcggtatggaccgctgggcggaggaaagactactacaccggcacccaggccacctacacctgcaccgaccattgcttcacaaccactcagtctccgacctgaagcctgtagacctgctgctggcggtgcagtccataccagaggactcgattttgcttgcgacatttatatatgggcacctttggcaggaacctgcggggtattgcttctttccctggtgatcacactgtattgtggtggaagtgggagcgaacgaacaatgccgcgcatcccgactctcaagaaccttgaggatcttgtcacggagtatcatggaaacttctccgcatggagcggcgtctcaaaaggcttggccgagtctcttcaaccagattactcagaaagattgtgcctggtctccgaaattccacctaaaggcggagccctcggcgaaggcccaggagcaagcccgtgcaaccagcactccccgtattgggcaccaccatgctacaccctgaagccagaaacc

SEQ ID NO: 36 - DNA, 765 bp, synthetic construct
  source 1..765: mol_type = other DNA
atgctgttattggtcacatcactgctcctctgtgaactgcctcatcccgcttttttattgattccctatccctacgatgtgcccgattacgcttgcggtggaagtagcgggctcgaaatagaagcagccttcctggaacaggaaaatactgcgctggagaccgaggtggcagaacttgagcaagaagtgcaacggctggagaacgaggtctctcaatacgaaacgcggtatggaccgctgggcggaggaaagactactacaccggcacccaggccacctacacctgcaccgaccattgcttcacaaccactcagtctccgacctgaagcctgtagacctgctgctggcggtgcagtccataccagaggactcgattttgcttgcgacatttatatatgggcacctttggcaggaacctgcggggtattgcttctttccctggtgatcacactgtattgtaatcacaggaacaggaggcgagtttgcaagtgcccccgaccagttgtcgaacgaacaatgccgcgcatcccgactctcaagaaccttgaggatcttgtcacggagtatcatggaaacttctccgcatggagcggcgtctcaaaaggcttggccgagtctcttcaaccagattactcagaaagattgtgcctggtctccgaaattccacctaaaggcggagccctcggcgaaggcccaggagcaagcccgtgcaaccagcactccccgtattgggcaccaccatgctacaccctgaagccagaaacc

SEQ ID NO: 37 - DNA, 738 bp, synthetic construct
  source 1..738: mol_type = other DNA
atgctgttattggtcacatcactgctcctctgtgaactgcctcatcccgcttttttattgattccctatccctacgatgtgcccgattacgcttgcggtggaagtagcgggctcgaaatagaagcagccttcctggaacaggaaaatactgcgctggagaccgaggtggcagaacttgagcaagaagtgcaacggctggagaacgaggtctctcaatacgaaacgcggtatggaccgctgggcggaggaaagactactacaccggcacccaggccacctacacctgcaccgaccattgcttcacaaccactcagtctccgacctgaagcctgtagacctgctgctggcggtgcagtccataccagaggactcgattttgcttgcgacatttatatatgggcacctttggcaggaacctgcggggtattgcttctttccctggtgatcacactgtattgtaaaagtcgccaaaccccaccggaacgaacaatgccgcgcatcccgactctcaagaaccttgaggatcttgtcacggagtatcatggaaacttctccgcatggagcggcgtctcaaaaggcttggccgagtctcttcaaccagattactcagaaagattgtgcctggtctccgaaattccacctaaaggcggagccctcggcgaaggcccaggagcaagcccgtgcaaccagcactccccgtattgggcaccaccatgctacaccctgaagccagaaacc

SEQ ID NO: 38 - DNA, 2118 bp, synthetic construct
  source 1..2118: mol_type = other DNA
atgcttctgctcgtgacaagcctgctgctgtgcgagctgccccaccctgcctttctgctgatccctgactacaaagacgacgatgacaagtgcggtggaaaaggcggaggcttggaaattagagcagcatttcttaggcgcagaaacaccgcgctccgaaccagagtagccgagctgagacaacgggtacaaagggcaaggaatcgcgtgagtcaatatcgaactcgatacggccccctcacaaccacaccagctcctagacccccaacgcccgcccccactatagcaagtcagcccctcagccttcgaccagaggcatgccggccagcggcaggaggagcggtacatactagagggttggactttgcgtgcgacatatatatctgggcaccgctggcggggacatgtggcgtactccttctgtcattggtaattactctttactgtaattgtcgcaacacagggccttggttgaaaaaagttctcaaatgcaatacgccggacccatctaaatttttcagtcagctcagctctgagcatgggggagatgtgcaaaaatggctcagctcccccttcccatctagtagcttcagcccgggcggtctcgctccggagatatcccccctggaggtcctggagagggacaaggtaacacaactgctcctccaacaggacaaagtcccggagcccgcgagtttgagctcaaaccactctctgacgtcttgtttcactaatcagggttacttttttttccatctcccggatgctctcgaaattgaggcttgtcaggtatacttcacttacgatccgtattccgaggaagatccggacgagggagtcgcgggtgctcctaccggctcaagtccccagccgctccagccattgagcggagaggacgacgcatattgtacgtttccgtctcgggacgacctcctcctcttctccccttccttgcttggaggtccaagtcctccgtcaacagcacctgggggaagtggtgccggtgaggagcggatgcctccatccttgcaggaacgggtaccgagggactgggaccctcagccacttgggcctccgacgcctggtgtgccagatctcgttgacttccagcccccgccggaattggttctgcgagaggctggtgaagaggtgccagatgctgggcctcgagaaggtgtttcatttccatggagtaggcctccgggacagggagagttccgcgcgctcaatgcacgccttcctctgaatactgatgcctacctgagtcttcaggagctccagggccaagacccaacacatctggtttcaattggagcaacaaacttctctctgctgaaacaagccggagatgtcgaagagaatcctggaccggggtcgacaatgctgttattggtcacatcactgctcctctgtgaactgcctcatcccgcttttttattgattccctatccctacgatgtgcccgattacgcttgcggtggaagtagcgggctcgaaatagaagcagccttcctggaacaggaaaatactgcgctggagaccgaggtggcagaacttgagcaagaagtgcaacggctggagaacgaggtctctcaatacgaaacgcggtatggaccgctgggcggaggaaagactactacaccggcacccaggccacctacacctgcaccgaccattgcttcacaaccactcagtctccgacctgaagcctgtagacctgctgctggcggtgcagtccataccagaggactcgattttgcttgcgacatttatatatgggcacctttggcaggaacctgcggggtattgcttctttccctggtgatcacactgtattgtaaaagtcgccaaaccccaccggaacgaacaatgccgcgcatcccgactctcaagaaccttgaggatcttgtcacggagtatcatggaaacttctccgcatggagcggcgtctcaaaaggcttggccgagtctcttcaaccagattactcagaaagattgtgcctggtctccgaaattccacctaaaggcggagccctcggcgaaggcccaggagcaagcccgtgcaaccagcactccccgtattgggcaccaccatgctacaccctgaagccagaaacc

SEQ ID NO: 39 - DNA, 2172 bp, synthetic construct
  source 1..2172: mol_type = other DNA
atgcttctgctcgtgacaagcctgctgctgtgcgagctgccccaccctgcctttctgctgatccctgactacaaagacgacgatgacaagtgcggtggaaaaggcggaggcttggaaattagagcagcatttcttaggcgcagaaacaccgcgctccgaaccagagtagccgagctgagacaacgggtacaaagggcaaggaatcgcgtgagtcaatatcgaactcgatacggccccctcacaaccacaccagctcctagacccccaacgcccgcccccactatagcaagtcagcccctcagccttcgaccagaggcatgccggccagcggcaggaggagcggtacatactagagggttggactttgcgtgcgacatatatatctgggcaccgctggcggggacatgtggcgtactccttctgtcattggtaattactctttactgtaattgtcgcaacacagggccttggttgaaaaaagttctcaaatgcaatacgccggacccatctaaatttttcagtcagctcagctctgagcatgggggagatgtgcaaaaatggctcagctcccccttcccatctagtagcttcagcccgggcggtctcgctccggagatatcccccctggaggtcctggagagggacaaggtaacacaactgctcctccaacaggacaaagtcccggagcccgcgagtttgagctcaaaccactctctgacgtcttgtttcactaatcagggttacttttttttccatctcccggatgctctcgaaattgaggcttgtcaggtatacttcacttacgatccgtattccgaggaagatccggacgagggagtcgcgggtgctcctaccggctcaagtccccagccgctccagccattgagcggagaggacgacgcatattgtacgtttccgtctcgggacgacctcctcctcttctccccttccttgcttggaggtccaagtcctccgtcaacagcacctgggggaagtggtgccggtgaggagcggatgcctccatccttgcaggaacgggtaccgagggactgggaccctcagccacttgggcctccgacgcctggtgtgccagatctcgttgacttccagcccccgccggaattggttctgcgagaggctggtgaagaggtgccagatgctgggcctcgagaaggtgtttcatttccatggagtaggcctccgggacagggagagttccgcgcgctcaatgcacgccttcctctgaatactgatgcctacctgagtcttcaggagctccagggccaagacccaacacatctggtttcaattggagcaacaaacttctctctgctgaaacaagccggagatgtcgaagagaatcctggaccggggtcgacaatgctgttattggtcacatcactgctcctctgtgaactgcctcatcccgcttttttattgattccctatccctacgatgtgcccgattacgcttgcggtggaagtagcgggctcgaaatagaagcagccttcctggaacaggaaaatactgcgctggagaccgaggtggcagaacttgagcaagaagtgcaacggctggagaacgaggtctctcaatacgaaacgcggtatggaccgctgggcggaggaaagactactacaccggcacccaggccacctacacctgcaccgaccattgcttcacaaccactcagtctccgacctgaagcctgtagacctgctgctggcggtgcagtccataccagaggactcgattttgcttgcgacatttatatatgggcacctttggcaggaacctgcggggtattgcttctttccctggtgatcacactgtattgtaaaagtcgccaaaccccaccgggtggccctggcgacgaaatggaagagtgtagccagcatctgcctggcggatccgaacgaacaatgccgcgcatcccgactctcaagaaccttgaggatcttgtcacggagtatcatggaaacttctccgcatggagcggcgtctcaaaaggcttggccgagtctcttcaaccagattactcagaaagattgtgcctggtctccgaaattccacctaaaggcggagccctcggcgaaggcccaggagcaagcccgtgcaaccagcactccccgtattgggcaccaccatgctacaccctgaagccagaaacc

SEQ ID NO: 40 - DNA, 795 bp, synthetic construct
  source 1..795: mol_type = other DNA
atgctgttattggtcacatcactgctcctctgtgaactgcctcatcccgcttttttattgattccctatccctacgatgtgcccgattacgcttgcggtggaagtagcgggctcgaaatagaagcagccttcctggaacaggaaaatactgcgctggagaccgaggtggcagaacttgagcaagaagtgcaacggctggagaacgaggtctctcaatacgaaacgcggtatggaccgctgggcggaggaaagactactacaccggcacccaggccacctacacctgcaccgaccattgcttcacaaccactcagtctccgacctgaagcctgtagacctgctgctggcggtgcagtccataccagaggactcgattttgcttgcgacatttatatatgggcacctttggcaggaacctgcggggtattgcttctttccctggtgatcacactgtattgtaaaagtcgccaaaccccaccgggtggccctggcgagtgtacgacaccttgctccggtagttggttgcctggcggatccgaacgaacaatgccgcgcatcccgactctcaagaaccttgaggatcttgtcacggagtatcatggaaacttctccgcatggagcggcgtctcaaaaggcttggccgagtctcttcaaccagattactcagaaagattgtgcctggtctccgaaattccacctaaaggcggagccctcggcgaaggcccaggagcaagcccgtgcaaccagcactccccgtattgggcaccaccatgctacaccctgaagccagaaacc

SEQ ID NO: 41 - DNA, 1155 bp, synthetic construct
  source 1..1155: mol_type = other DNA
atgggaactagcctcctgtgttggatggcgctttgcctgctcggagcggatcacgccgatgcgtgtccttatagcaacccttctctctgcagtggtggaggcgggagcgagttgccgactcagggcactttttcaaacgtaagcacaaatgtaagccctgccaagcctaccacgactgcttgtccctacagcaatccgagtctgtgttcaggaggaggagggtctccggcccctcgaccgcctacccccgcaccaacgattgcctcacagcccctcagtttgcgacccgaagcatgccgacctgcggcaggaggtgctgttcatacgcggggcttggactttgcctgtgacatttatatttgggctccactcgcaggcacatgcggcgtgctgttgctcagtctcgtcataacattgtattgcaatcacaggaacaggaggcgagtttgcaagtgcccccgaccagttgtcggcagttctgggaattcctctggaggttctacaggctgcgtggtcatagtgggcaggatcgtcttgtccggatccggcactagtgcgcccatcacggcgtacgcccagcagacgagaggcctcctagggtgtataatcaccagcctgactggccgggacaaaaaccaagtggagggtgaggtccagatcatgtcaactgctacccaaaccttcctggcaacgtgcatcaatggggtatgctggaccgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatggcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagagacaaccatgagatccccggtgttcacggacaactcctctccaccagcagtcaccctgacgcacaacgcgtct

SEQ ID NO: 42 - DNA, 2595 bp, synthetic construct
  source 1..2595: mol_type = other DNA
atgcttctgctcgtgacaagcctgctgctgtgcgagctgccccaccctgcctttctgctgatccctggaggatccgactacaaagacgacgatgacaagtgcggtggaaaaggcggaggcttggaaattagagcagcatttcttaggcgcagaaacaccgcgctccgaaccagagtagccgagctgagacaacgggtacaaagggcaaggaatcgcgtgagtcaatatcgaactcgatacggccccctcacaaccacaccagctcctagacccccaacgcccgcccccactatagcaagtcagcccctcagccttcgaccagaggcatgccggccagcggcaggaggagcggtacatactagagggttggactttgcgtgcgacatatatatctgggcaccgctggcggggacatgtggcgtactccttctgtcattggtaattactctttactgtaaaccaggtggccctggcgagtgtacgacaccttgctccggtagttggttggcggccgcaaattgtcgcaacacagggccttggttgaaaaaagttctcaaatgcaatacgccggacccatctaaatttttcagtcagctcagctctgagcatgggggagatgtgcaaaaatggctcagctcccccttcccatctagtagcttcagcccgggcggtctcgctccggagatatcccccctggaggtcctggagagggacaaggtaacacaactgctcctccaacaggacaaagtcccggagcccgcgagtttgagctcaaaccactctctgacgtcttgtttcactaatcagggttacttttttttccatctcccggatgctctcgaaattgaggcttgtcaggtatacttcacttacgatccgtattccgaggaagatccggacgagggagtcgcgggtgctcctaccggctcaagtccccagccgctccagccattgagcggagaggacgacgcatattgtacgtttccgtctcgggacgacctcctcctcttctccccttccttgcttggaggtccaagtcctccgtcaacagcacctgggggaagtggtgccggtgaggagcggatgcctccatccttgcaggaacgggtaccgagggactgggaccctcagccacttgggcctccgacgcctggtgtgccagatctcgttgacttccagcccccgccggaattggttctgcgagaggctggtgaagaggtgccagatgctgggcctcgagaaggtgtttcatttccatggagtaggcctccgggacagggagagttccgcgcgctcaatgcacgccttcctctgaatactgatgcctacctgagtcttcaggagctccagggccaagacccaacacatctggttgggtcgacagcaacaaacttctctctgctgaaacaagccggagatgtcgaagagaatcctggaccgatgggaactagcctcctgtgttggatggcgctttgcctgctcggagcggatcacgccgatgcgtgtccttatagcaacccttctctctgcagtggtggaggcgggagcgagttgccgactcagggcactttttcaaacgtaagcacaaatgtaagccctgccaagcctaccacgactgcttgtccctacagcaatccgagtctgtgttcaggaggaggagggtctccggcccctcgaccgcctacccccgcaccaacgattgcctcacagcccctcagtttgcgacccgaagcatgccgacctgcggcaggaggtgctgttcatacgcggggcttggactttgcctgtgacatttatatttgggctccactcgcaggcacatgcggcgtgctgttgctcagtctcgtcataacattgtattgcaatcacaggaacaggaggcgagtttgcaagtgcccccgaccagttgtcggcagttctgggaattcctctggaggttctacaggctgcgtggtcatagtgggcaggatcgtcttgtccggatccggcactagtgcgcccatcacggcgtacgcccagcagacgagaggcctcctagggtgtataatcaccagcctgactggccgggacaaaaaccaagtggagggtgaggtccagatcatgtcaactgctacccaaaccttcctggcaacgtgcatcaatggggtatgctggaccgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatggcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagagacaaccatgagatccccggtgttcacggacaactcctctccaccagcagtcaccctgacgcacaacgcgtct

SEQ ID NO: 43 - DNA, 2016 bp, synthetic construct
  source 1..2016: mol_type = other DNA
atgctgttattggtcacatcactgctcctctgtgaactgcctcatcccgcttttttattgattccctatccctacgatgtgcccgattacgcttgcggtggaagtagcgggctcgaaatagaagcagccttcctggaacaggaaaatactgcgctggagaccgaggtggcagaacttgagcaagaagtgcaacggctggagaacgaggtctctcaatacgaaacgcggtatggaccgctgggcggaggaaagactactacaccggcacccaggccacctacacctgcaccgaccattgcttcacaaccactcagtctccgacctgaagcctgtagacctgctgctggcggtgcagtccataccagaggactcgattttgcttgcgacatttatatatgggcacctttggcaggaacctgcggggtattgcttctttccctggtgatcacactgtattgtaaaagtcgccaaaccccaccgggtggccctggcgagtgtacgacaccttgctccggtagttggttgcctggcggatccgaacgaacaatgccgcgcatcccgactctcaagaaccttgaggatcttgtcacggagtatcatggaaacttctccgcatggagcggcgtctcaaaaggcttggccgagtctcttcaaccagattactcagaaagattgtgcctggtctccgaaattccacctaaaggcggagccctcggcgaaggcccaggagcaagcccgtgcaaccagcactccccgtattgggcaccaccatgctacaccctgaagccagaaaccgggtcgacagcaacaaacttctctctgctgaaacaagccggagatgtcgaagagaatcctggaccgatgggaactagcctcctgtgttggatggcgctttgcctgctcggagcggatcacgccgatgcgtgtccttatagcaacccttctctctgcagtggtggaggcgggagcgagttgccgactcagggcactttttcaaacgtaagcacaaatgtaagccctgccaagcctaccacgactgcttgtccctacagcaatccgagtctgtgttcaggaggaggagggtctccggcccctcgaccgcctacccccgcaccaacgattgcctcacagcccctcagtttgcgacccgaagcatgccgacctgcggcaggaggtgctgttcatacgcggggcttggactttgcctgtgacatttatatttgggctccactcgcaggcacatgcggcgtgctgttgctcagtctcgtcataacattgtattgcaatcacaggaacaggaggcgagtttgcaagtgcccccgaccagttgtcggcagttctgggaattcctctggaggttctacaggctgcgtggtcatagtgggcaggatcgtcttgtccggatccggcactagtgcgcccatcacggcgtacgcccagcagacgagaggcctcctagggtgtataatcaccagcctgactggccgggacaaaaaccaagtggagggtgaggtccagatcatgtcaactgctacccaaaccttcctggcaacgtgcatcaatggggtatgctggaccgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatggcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagagacaaccatgagatccccggtgttcacggacaactcctctccaccagcagtcaccctgacgcacaacgcgtct

SEQ ID NO: 44 - DNA, 3378 bp, synthetic construct
  source 1..3378: mol_type = other DNA
atgctgttattggtcacatcactgctcctctgtgaactgcctcatcccgcttttttattgattccctatccctacgatgtgcccgattacgcttgcggtggaagtagcgggctcgaaatagaagcagccttcctggaacaggaaaatactgcgctggagaccgaggtggcagaacttgagcaagaagtgcaacggctggagaacgaggtctctcaatacgaaacgcggtatggaccgctgggcggaggaaagactactacaccggcacccaggccacctacacctgcaccgaccattgcttcacaaccactcagtctccgacctgaagcctgtagacctgctgctggcggtgcagtccataccagaggactcgattttgcttgcgacatttatatatgggcacctttggcaggaacctgcggggtattgcttctttccctggtgatcacactgtattgtaaaagtcgccaaaccccaccgggtggccctggcgagtgtacgacaccttgctccggtagttggttgcctggcggatccgaacgaacaatgccgcgcatcccgactctcaagaaccttgaggatcttgtcacggagtatcatggaaacttctccgcatggagcggcgtctcaaaaggcttggccgagtctcttcaaccagattactcagaaagattgtgcctggtctccgaaattccacctaaaggcggagccctcggcgaaggcccaggagcaagcccgtgcaaccagcactccccgtattgggcaccaccatgctacaccctgaagccagaaaccgggtcgacagcaacaaacttctctctgctgaaacaagccggagatgtcgaagagaatcctggaccgatgggaactagcctcctgtgttggatggcgctttgcctgctcggagcggatcacgccgatgcgtgtccttatagcaacccttctctctgcagtggtggaggcgggagcgagttgccgactcagggcactttttcaaacgtaagcacaaatgtaagccctgccaagcctaccacgactgcttgtccctacagcaatccgagtctgtgttcaggaggaggagggtctccggcccctcgaccgcctacccccgcaccaacgattgcctcacagcccctcagtttgcgacccgaagcatgccgacctgcggcaggaggtgctgttcatacgcggggcttggactttgcctgtgacatttatatttgggctccactcgcaggcacatgcggcgtgctgttgctcagtctcgtcataacattgtattgcaatcacaggaacaggaggcgagtttgcaagtgcccccgaccagttgtcggcagttctgggaattcctctggaggttctacaggctgcgtggtcatagtgggcaggatcgtcttgtccggatccggcactagtgcgcccatcacggcgtacgcccagcagacgagaggcctcctagggtgtataatcaccagcctgactggccgggacaaaaaccaagtggagggtgaggtccagatcatgtcaactgctacccaaaccttcctggcaacgtgcatcaatggggtatgctggaccgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatggcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagagacaaccatgagatccccggtgttcacggacaactcctctccaccagcagtcaccctgacgcacaacgcgtctgccactaacttttcactcttgaagcaggctggcgatgtagaagagaaccctggacctatgcttctgctcgtgacaagcctgctgctgtgcgagctgccccaccctgcctttctgctgatccctgactacaaagacgacgatgacaagtgcggtggaaaaggcggaggcttggaaattagagcagcatttcttaggcgcagaaacaccgcgctccgaaccagagtagccgagctgagacaacgggtacaaagggcaaggaatcgcgtgagtcaatatcgaactcgatacggccccctcacaaccacaccagctcctagacccccaacgcccgcccccactatagcaagtcagcccctcagccttcgaccagaggcatgccggccagcggcaggaggagcggtacatactagagggttggactttgcgtgcgacatatatatctgggcaccgctggcggggacatgtggcgtactccttctgtcattggtaattactctttactgtaattgtcgcaacacagggccttggttgaaaaaagttctcaaatgcaatacgccggacccatctaaatttttcagtcagctcagctctgagcatgggggagatgtgcaaaaatggctcagctcccccttcccatctagtagcttcagcccgggcggtctcgctccggagatatcccccctggaggtcctggagagggacaaggtaacacaactgctcctccaacaggacaaagtcccggagcccgcgagtttgagctcaaaccactctctgacgtcttgtttcactaatcagggttacttttttttccatctcccggatgctctcgaaattgaggcttgtcaggtatacttcacttacgatccgtattccgaggaagatccggacgagggagtcgcgggtgctcctaccggctcaagtccccagccgctccagccattgagcggagaggacgacgcatattgtacgtttccgtctcgggacgacctcctcctcttctccccttccttgcttggaggtccaagtcctccgtcaacagcacctgggggaagtggtgccggtgaggagcggatgcctccatccttgcaggaacgggtaccgagggactgggaccctcagccacttgggcctccgacgcctggtgtgccagatctcgttgacttccagcccccgccggaattggttctgcgagaggctggtgaagaggtgccagatgctgggcctcgagaaggtgtttcatttccatggagtaggcctccgggacagggagagttccgcgcgctcaatgcacgccttcctctgaatactgatgcctacctgagtcttcaggagctccagggccaagacccaacacatctggtt

SEQ ID NO: 45 - DNA, 1530 bp, synthetic construct
  source 1..1530: mol_type = other DNA
atgcttctgctcgtgacaagcctgctgctgtgcgagctgccccaccctgcctttctgctgatccctggaggatcctatccctacgatgtgcccgattacgctggtggaacaaccacaccagctcctagacccccaacgcccgcccccactatagcaagtcagcccctcagccttcgaccagaggcatgccggccagcggcaggaggagcggtacatactagagggttggactttgcgtgcgacatatatatctgggcaccgctggcggggacatgtggcgtactccttctgtcattggtaattactctttactgtaaaggtggcggaggatctggcggcgcggccgcaaaaagtcgccaaaccccaccggaacgaacaatgccgcgcatcccgactctcaagaaccttgaggatcttgtcacggagtatcatggaaacttctccgcatggagcggcgtctcaaaaggcttggccgagtctcttcaaccagattactcagaaagattgtgcctggtctccgaaattccacctaaaggcggagccctcggcgaaggcccaggagcaagcccgtgcaaccagcactccccgtattgggcaccaccatgctacaccctgaagccagaaaccggcagttctgggaattcctctggaggttctgggtcgacaggttccaattgtcgcaacacagggccttggttgaaaaaagttctcaaatgcaatacgccggacccatctaaatttttcagtcagctcagctctgagcatgggggagatgtgcaaaaatggctcagctcccccttcccatctagtagcttcagcccgggcggtctcgctccggagatatcccccctggaggtcctggagagggacaaggtaacacaactgctcctccaacaggacaaagtcccggagcccgcgagtttgagctcaaaccactctctgacgtcttgtttcactaatcagggttacttttttttccatctcccggatgctctcgaaattgaggcttgtcaggtatacttcacttacgatccgtattccgaggaagatccggacgagggagtcgcgggtgctcctaccggctcaagtccccagccgctccagccattgagcggagaggacgacgcatattgtacgtttccgtctcgggacgacctcctcctcttctccccttccttgcttggaggtccaagtcctccgtcaacagcacctgggggaagtggtgccggtgaggagcggatgcctccatccttgcaggaacgggtaccgagggactgggaccctcagccacttgggcctccgacgcctggtgtgccagatctcgttgacttccagcccccgccggaattggttctgcgagaggctggtgaagaggtgccagatgctgggcctcgagaaggtgtttcatttccatggagtaggcctccgggacagggagagttccgcgcgctcaatgcacgccttcctctgaatactgatgcctacctgagtcttcaggagctccagggccaagacccaacacatctggtt

SEQ ID NO: 46 - DNA, 1509 bp, synthetic construct
  source 1..1509: mol_type = other DNA
atgcttctgctcgtgacaagcctgctgctgtgcgagctgccccaccctgcctttctgctgatccctggaggatcctatccctacgatgtgcccgattacgctggtggaacaaccacaccagctcctagacccccaacgcccgcccccactatagcaagtcagcccctcagccttcgaccagaggcatgccggccagcggcaggaggagcggtacatactagagggttggactttgcgtgcgacatatatatctgggcaccgctggcggggacatgtggcgtactccttctgtcattggtaattactctttactgtaaaggtggcggaggatctggcggcgcggccgcaaattgtcgcaacacagggccttggttgaaaaaagttctcaaatgcaatacgccggacccatctaaatttttcagtcagctcagctctgagcatgggggagatgtgcaaaaatggctcagctcccccttcccatctagtagcttcagcccgggcggtctcgctccggagatatcccccctggaggtcctggagagggacaaggtaacacaactgctcctccaacaggacaaagtcccggagcccgcgagtttgagctcaaaccactctctgacgtcttgtttcactaatcagggttacttttttttccatctcccggatgctctcgaaattgaggcttgtcaggtatacttcacttacgatccgtattccgaggaagatccggacgagggagtcgcgggtgctcctaccggctcaagtccccagccgctccagccattgagcggagaggacgacgcatattgtacgtttccgtctcgggacgacctcctcctcttctccccttccttgcttggaggtccaagtcctccgtcaacagcacctgggggaagtggtgccggtgaggagcggatgcctccatccttgcaggaacgggtaccgagggactgggaccctcagccacttgggcctccgacgcctggtgtgccagatctcgttgacttccagcccccgccggaattggttctgcgagaggctggtgaagaggtgccagatgctgggcctcgagaaggtgtttcatttccatggagtaggcctccgggacagggagagttccgcgcgctcaatgcacgccttcctctgaatactgatgcctacctgagtcttcaggagctccagggccaagacccaacacatctggttggcagttctgggaattcctctggaggttctgggtcgacaggttccgaacgaacaatgccgcgcatcccgactctcaagaaccttgaggatcttgtcacggagtatcatggaaacttctccgcatggagcggcgtctcaaaaggcttggccgagtctcttcaaccagattactcagaaagattgtgcctggtctccgaaattccacctaaaggcggagccctcggcgaaggcccaggagcaagcccgtgcaaccagcactccccgtattgggcaccaccatgctacaccctgaagccagaaacc

SEQ ID NO: 47 - DNA, 1452 bp, synthetic construct
  source 1..1452: mol_type = other DNA
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 48 - DNA, 2832 bp, synthetic construct
  source 1..2832: mol_type = other DNA
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaacaacaacaccagctcctagacctccaactcctgctcctacaatcgccagccagcctctgtctctgaggcctgaagcttgtagacctgctgctggcggagccgtgcataccagaggactggatttcgcctgcgacatctacatttgggcccctctggctggaacatgtggcgtgctgctgctgtctctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttctctagatctgccgacgctcccgcctacaagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggccacgatggactgtatcagggcctgagcacagctacaaaggatacctatgatgccctgcacatgcaggcccttccacctagatcaattggagcaacaaacttctctctgctgaaacaagccggagatgtcgaagagaatcctggaccggggtcgacaatgcttctgctcgtgacaagcctgctgctgtgcgagctgccccaccctgcctttctgctgatccctgactacaaagacgacgatgacaagtgcggtggaaaaggcggaggcttggaaattagagcagcatttcttaggcgcagaaacaccgcgctccgaaccagagtagccgagctgagacaacgggtacaaagggcaaggaatcgcgtgagtcaatatcgaactcgatacggccccctcacaaccacaccagctcctagacccccaacgcccgcccccactatagcaagtcagcccctcagccttcgaccagaggcatgccggccagcggcaggaggagcggtacatactagagggttggactttgcgtgcgacatatatatctgggcaccgctggcggggacatgtggcgtactccttctgtcattggtaattactctttactgtaattgtcgcaacacagggccttggttgaaaaaagttctcaaatgcaatacgccggacccatctaaatttttcagtcagctcagctctgagcatgggggagatgtgcaaaaatggctcagctcccccttcccatctagtagcttcagcccgggcggtctcgctccggagatatcccccctggaggtcctggagagggacaaggtaacacaactgctcctccaacaggacaaagtcccggagcccgcgagtttgagctcaaaccactctctgacgtcttgtttcactaatcagggttacttttttttccatctcccggatgctctcgaaattgaggcttgtcaggtatacttcacttacgatccgtattccgaggaagatccggacgagggagtcgcgggtgctcctaccggctcaagtccccagccgctccagccattgagcggagaggacgacgcatattgtacgtttccgtctcgggacgacctcctcctcttctccccttccttgcttggaggtccaagtcctccgtcaacagcacctgggggaagtggtgccggtgaggagcggatgcctccatccttgcaggaacgggtaccgagggactgggaccctcagccacttgggcctccgacgcctggtgtgccagatctcgttgacttccagcccccgccggaattggttctgcgagaggctggtgaagaggtgccagatgctgggcctcgagaaggtgtttcatttccatggagtaggcctccgggacagggagagttccgcgcgctcaatgcacgccttcctctgaatactgatgcctacctgagtcttcaggagctccagggccaagacccaacacatctggtt

SEQ ID NO: 49 - DNA, 1524 bp, synthetic construct
  source 1..1524: mol_type = other DNA
atgctgttattggtcacatcactgctcctctgtgaactgcctcatcccgcttttttattgattccctatccctacgatgtgcccgattacgcttgcggtggaagtagcgggctcgaaatagaagcagccttcctggaacaggaaaatactgcgctggagaccgaggtggcagaacttgagcaagaagtgcaacggctggagaacgaggtctctcaatacgaaacgcggtatggaccgctgggcggaggaaagactactacaccggcacccaggccacctacacctgcaccgaccattgcttcacaaccactcagtctccgacctgaagcctgtagacctgctgctggcggtgcagtccataccagaggactcgattttgcttgcgacatttatatatgggcacctttggcaggaacctgcggggtattgcttctttccctggtgatcacactgtattgtaaaagtcgccaaaccccaccgggtggccctggcgagtgtacgacaccttgctccggtagttggttgcctggcggatccgaacgaacaatgccgcgcatcccgactctcaagaaccttgaggatcttgtcacggagtatcatggaaacttctccgcatggagcggcgtctcaaaaggcttggccgagtctcttcaaccagattactcagaaagattgtgcctggtctccgaaattccacctaaaggcggagccctcggcgaaggcccaggagcaagcccgtgcaaccagcactccccgtattgggcaccaccatgctacaccctgaagccagaaaccgggtcgacaggtggaggatctggcggcggaagcggaggcggatctggcagttctgggaattcctctggaggttctacaggctgcgtggtcatagtgggcaggatcgtcttgtccggatccggcactagtgcgcccatcacggcgtacgcccagcagacgagaggcctcctagggtgtataatcaccagcctgactggccgggacaaaaaccaagtggagggtgaggtccagatcatgtcaactgctacccaaaccttcctggcaacgtgcatcaatggggtatgctggaccgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatggcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagagacaaccatgagatccccggtgttcacggacaactcctctccaccagcagtcaccctgacgcacaacgcgtct

SEQ ID NO: 50 - DNA, 1488 bp, synthetic construct
  source 1..1488: mol_type = other DNA
atgctgttattggtcacatcactgctcctctgtgaactgcctcatcccgcttttttattgattccctatccctacgatgtgcccgattacgcttgcggtggaagtagcgggctcgaaatagaagcagccttcctggaacaggaaaatactgcgctggagaccgaggtggcagaacttgagcaagaagtgcaacggctggagaacgaggtctctcaatacgaaacgcggtatggaccgctgggcggaggaaagactactacaccggcacccaggccacctacacctgcaccgaccattgcttcacaaccactcagtctccgacctgaagcctgtagacctgctgctggcggtgcagtccataccagaggactcgattttgcttgcgacatttatatatgggcacctttggcaggaacctgcggggtattgcttctttccctggtgatcacactgtattgtaaaagtcgccaaaccccaccgggtggccctggcgagtgtacgacaccttgctccggtagttggttgcctggcggatccgaacgaacaatgccgcgcatcccgactctcaagaaccttgaggatcttgtcacggagtatcatggaaacttctccgcatggagcggcgtctcaaaaggcttggccgagtctcttcaaccagattactcagaaagattgtgcctggtctccgaaattccacctaaaggcggagccctcggcgaaggcccaggagcaagcccgtgcaaccagcactccccgtattgggcaccaccatgctacaccctgaagccagaaaccgggtcgacaggcagttctgggaattcctctggaggttctacaggctgcgtggtcatagtgggcaggatcgtcttgtccggatccggcactagtgcgcccatcacggcgtacgcccagcagacgagaggcctcctagggtgtataatcaccagcctgactggccgggacaaaaaccaagtggagggtgaggtccagatcatgtcaactgctacccaaaccttcctggcaacgtgcatcaatggggtatgctggaccgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatggcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagagacaaccatgagatccccggtgttcacggacaactcctctccaccagcagtcaccctgacgcacaacgcgtct

SEQ ID NO: 51 - DNA, 1845 bp, synthetic construct
  source 1..1845: mol_type = other DNA
atgcttctgctcgtgacaagcctgctgctgtgcgagctgccccaccctgcctttctgctgatccctgactacaaagacgacgatgacaagtgcggtggaaaaggcggaggcttggaaattagagcagcatttcttaggcgcagaaacaccgcgctccgaaccagagtagccgagctgagacaacgggtacaaagggcaaggaatcgcgtgagtcaatatcgaactcgatacggccccctcacaaccacaccagctcctagacccccaacgcctgcccccactatagcaagtcagcctctcagccttcgaccagaggcatgccggccagcggcaggaggagcggtacatactagagggttggactttgcgtgcgacatatatatctgggcaccgctggctgggacatgtggcgtactccttctgtcattggtaattactctttactgtaaaaagaggataaagccgatagtttggccgtccctgccagatcacaagaagacacttgagcatttgtgtaagaagccaaggaagaatctcaacgtgtcattcaaccctgaaagtttccttgactgccagatacatagagttgacgatatacaggctagagatgaggtggagggcttcttgcaagataccttcccgcagcagttggaggaatccgagaaacaaaggctcggcggtgacgtccaatcacccaactgtccaagcgaggacgtggtcataactccggagtccttcggacgagattctagccttacgtgccttgccggcaacgttagtgcctgtgatgcaccgatactcagcagcagccggtctctcgactgtcgggaatctggtaagaatggccctcatgtatatcaggacctgctgctttcactcggaaccactaattccacattgccacctccatttagcttgcaatctgggatcttgactctcaacccggtggcacagggtcagcctatcctgacgtcccttggcagtaaccaagaagaagcctacgtgactatgagcagcttctaccagaatcaatcaattggagcaacaaacttctctctgctgaaacaagccggagatgtcgaagagaatcctggaccggggtcgacaatgctgttattggtcacatcactgctcctctgtgaactgcctcatcccgcttttttattgattccctatccctacgatgtgcccgattacgcttgcggtggaagtagcgggctcgaaatagaagcagccttcctggaacaggaaaatactgcgctggagaccgaggtggcagaacttgagcaagaagtgcaacggctggagaacgaggtctctcaatacgaaacgcggtatggaccgctgggcggaggaaagactactacaccggcacccaggccacctacacctgcaccgaccattgcttcacaaccactcagtctccgacctgaagcctgtagacctgctgctggcggtgcagtccataccagaggactcgattttgcttgcgacatttatatatgggcacctttggcaggaacctgcggggtattgcttctttccctggtgatcacactgtattgtaaaagtcgccaaaccccaccggaacgaacaatgccgcgcatcccgactctcaagaaccttgaggatcttgtcacggagtatcatggaaacttctccgcatggagcggcgtctcaaaaggcttggccgagtctcttcaaccagattactcagaaagattgtgcctggtctccgaaattccacctaaaggcggagccctcggcgaaggcccaggagcaagcccgtgcaaccagcactccccgtattgggcaccaccatgctacaccctgaagccagaaacc

SEQ ID NO: 52 - DNA, 2664 bp, synthetic construct
  source 1..2664: mol_type = other DNA
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaacaacaacaccagctcctagacctccaactcctgctcctacaatcgccagccagcctctgtctctgaggcctgaagcttgtagacctgctgctggcggagccgtgcataccagaggactggatttcgcctgcgacatctacatttgggcccctctggctggaacatgtggcgtgctgctgctgtctctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttctctagatctgccgacgctcccgcctacaagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggccacgatggactgtatcagggcctgagcacagctacaaaggatacctatgatgccctgcacatgcaggcccttccacctagatcaattggagcaacaaacttctctctgctgaaacaagccggagatgtcgaagagaatcctggaccggggtcgacaatgcttctgctcgtgacaagcctgctgctgtgcgagctgccccaccctgcctttctgctgatccctgactacaaagacgacgatgacaagtgcggtggaaaaggcggaggcttggaaattagagcagcatttcttaggcgcagaaacaccgcgctccgaaccagagtagccgagctgagacaacgggtacaaagggcaaggaatcgcgtgagtcaatatcgaactcgatacggccccctcacaaccacaccagctcctagacccccaacgcctgcccccactatagcaagtcagcctctcagccttcgaccagaggcatgccggccagcggcaggaggagcggtacatactagagggttggactttgcgtgcgacatatatatctgggcaccgctggctgggacatgtggcgtactccttctgtcattggtaattactctttactgtaagttgtctccacgggtaaagagaattttctaccagaacgtcccatcacccgctatgttctttcaaccactctactcagttcacaacgggaatttccaaacatggatgggggctcacggtgcaggtgtccttcttagccaagattgtgccggtacaccacagggtgcacttgagccttgtgtgcaggaagcgacggcgctgctgacttgtggtccagccagaccttggaagtcagttgccctggaagaggagcaagaggggccgggaacacgattgcccgggaatctgagttcagaggatgtattgcccgctggctgcacagagtggagagtccagacactcgcatacctgccgcaggaagactgggcacctacgtctttgacaaggcccgcaccaccagattccgaggggagccgatcttcatccagttccagctcatccaataataataattactgtgcccttggatgttatgggggttggcatctttcagctttgccagggaacacacaaagcagtgggcccatacctgctctggcctgcggcttgagttgcgaccaccaaggtcttgagactcagcagggtgtagcttgggtgctcgccgggcattgtcaacgacctggtctgcacgaagatttgcagggtatgctcctgcctagtgttctgagtaaggcgaggtcttggactttc

SEQ ID NO: 53 - DNA, 2559 bp, synthetic construct
  source 1..2559: mol_type = other DNA
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaacaacaacaccagctcctagacctccaactcctgctcctacaatcgccagccagcctctgtctctgaggcctgaagcttgtagacctgctgctggcggagccgtgcataccagaggactggatttcgcctgcgacatctacatttgggcccctctggctggaacatgtggcgtgctgctgctgtctctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttctctagatctgccgacgctcccgcctacaagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggccacgatggactgtatcagggcctgagcacagctacaaaggatacctatgatgccctgcacatgcaggcccttccacctagatcaattggagcaacaaacttctctctgctgaaacaagccggagatgtcgaagagaatcctggaccggggtcgacaatgcttctgctcgtgacaagcctgctgctgtgcgagctgccccaccctgcctttctgctgatccctgactacaaagacgacgatgacaagtgcggtggaaaaggcggaggcttggaaattagagcagcatttcttaggcgcagaaacaccgcgctccgaaccagagtagccgagctgagacaacgggtacaaagggcaaggaatcgcgtgagtcaatatcgaactcgatacggccccctcacaaccacaccagctcctagacccccaacgcctgcccccactatagcaagtcagcctctcagccttcgaccagaggcatgccggccagcggcaggaggagcggtacatactagagggttggactttgcgtgcgacatatatatctgggcaccgctggctgggacatgtggcgtactccttctgtcattggtaattactctttactgtaaaaagaggataaagccgatagtttggccgtccctgccagatcacaagaagacacttgagcatttgtgtaagaagccaaggaagaatctcaacgtgtcattcaaccctgaaagtttccttgactgccagatacatagagttgacgatatacaggctagagatgaggtggagggcttcttgcaagataccttcccgcagcagttggaggaatccgagaaacaaaggctcggcggtgacgtccaatcacccaactgtccaagcgaggacgtggtcataactccggagtccttcggacgagattctagccttacgtgccttgccggcaacgttagtgcctgtgatgcaccgatactcagcagcagccggtctctcgactgtcgggaatctggtaagaatggccctcatgtatatcaggacctgctgctttcactcggaaccactaattccacattgccacctccatttagcttgcaatctgggatcttgactctcaacccggtggcacagggtcagcctatcctgacgtcccttggcagtaaccaagaagaagcctacgtgactatgagcagcttctaccagaatcaa

SEQ ID NO: 54 - protein, 16 aa, synthetic construct
NHRNRRRVCKCPRPVV

SEQ ID NO: 55 - protein, 7 aa, synthetic construct
KSRQTPP

SEQ ID NO: 56 - protein, 33 aa, synthetic construct
GGGGSGGGGSGGGGSPGDEMEECSQHLPGAGSS

SEQ ID NO: 57 - protein, 19 aa, synthetic construct
PGGPGDEMEECSQHLPGGS

SEQ ID NO: 58 - protein, 7 aa, synthetic construct
ENLYFQS

SEQ ID NO: 59 - protein, 236 aa, synthetic construct
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMN

SEQ ID NO: 60 - protein, 186 aa, synthetic construct
APITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTD

SEQ ID NO: 61 - protein, 186 aa, synthetic construct
APITAYAQQTRGLLGCIITSLTGRDKNQVEGEVQIMSTATQTFLATCINGVCWTVYHGAGTRTIASPKGPVIQMYTNVDQDLVGWPAPQGSRSLTPCTCGSSDLYLVTRHADVIPVRRRGDGRGSLLSPRPISYLKGSSGGPLLCPAGHAVGLFRAAVCTRGVAKAVDFIPVENLETTMRSPVFTD

SEQ ID NO: 62 - protein, 186 aa, synthetic construct
APITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTD

SEQ ID NO: 63 - protein, 15 aa, synthetic construct
GGGGSGGGGSGGGGS